(19) United States
(12) Patent Application Publication
(10) Pub. No.: US 2014/0315973 A1
(43) Pub. Date: Oct. 23, 2014

(54) PARP-1 INHIBITORS

(71) Applicants: Ee Chee Ren, Singapore (SG); Hui Ling Ko, Singapore (SG)

(72) Inventors: Ee Chee Ren, Singapore (SG); Hui Ling Ko, Singapore (SG)

(73) Assignee: AGENCY FOR SCIENCE, TECHNOLOGY AND RESEARCH, Singapore (SG)

(21) Appl. No.: 13/877,972

(22) PCT Filed: Oct. 7, 2011
(86) PCT No.: PCT/SG11/00351

§ 371 Date: May 20, 2014

(30) Foreign Application Priority Data
Oct. 7, 2010 (SG) 201007366-6

(51) Int. Cl. C12N 15/113; A61K 45/06; C12Q 1/48; A61K 31/713

(52) U.S. Cl. CPC C12N 15/1137; A61K 31/713; A61K 45/06; C12Q 1/48
USPC 514/44 A; 536/24.5; 435/188; 435/184; 435/6.1; 506/9; 435/6.12; 435/6.11

(57) ABSTRACT

We describe a nucleic acid comprising the sequence RNNWCAAA, in which R is independently G or A, N is independently T, C, G or A and W is independently T or A, suitable for the treatment or prevention of hepatitis B or cancer. N at position 3 may be C, A or T, preferably A or T, more preferably T; N at position 2 may be C; W at position 4 may be T; and R at position 1 may be A. The nucleic acid may have the sequence ACATCAAA or ACTTCAAA.

### FIELD

The present invention relates to the fields of medicine, cell biology, molecular biology and genetics.

### BACKGROUND

More than 350 million people world-wide are chronically infected with HBV. These people have increased risk of developing liver-associated diseases such as cirrhosis and HCC.

Treatment of HBV infection relies on the long-term usage of drugs. These include immune-modulators such as interferons, which is not well-tolerated as a result of severe side effects in many individuals and the need for injections, as well as the use of nucleoside analogues that inhibit viral replication by targeting the HBV polymerase [1]. Nucleoside or nucleotide analogues, though better tolerated, are not the ideal drugs of choice, as they have resulted in the production of multiple resistant strains with long-term usage. While new analogues are available, with time, the generation of escape mutants is probably inevitable. Designing new analogues is clearly not the long-term solution.

Furthermore, the persistence of low levels of HBV DNA long after therapy suggests that HBV replication may re-activate when conditions are once again optimal. Reports indicating that vaccination is ineffective against certain genotypes such as the highly lethal genotype F also raise concern that current vaccination strategies may be ineffective in containing their spread [2-5].

There is therefore a need in the art for alternative means of controlling HBV replication.

### SUMMARY

According to a 1^{st} aspect of the present invention, we provide use of a nucleic acid in the preparation of a medicament for the treatment or prevention of hepatitis B or cancer. The nucleic acid may comprise the sequence RNNWCAAA.

R may be independently G or A. N may be independently T, C, G or A. W may be independently T or A. N at position 3 may be C, A or T, preferably A or T, more preferably T.

N at position 2 may be C. W at position 4 may be T. R at position 1 may be A.

The nucleic acid may have the sequence ACATCAAA or ACTTCAAA.

The cancer may comprise breast cancer. The cancer cells may be BRCA1 and/or BRCA2-deficient.

The medicament may further comprise a cytotoxic agent. The cytotoxic agent may comprise ionising radiation. The cytotoxic agent may comprise ABT-888 (Abbott). The cytotoxic agent may comprise AG014699 (Pfizer). The cytotoxic agent may comprise AZD2281 (olaparib, AstraZeneca). The cytotoxic agent may comprise BSI-201 (Sanofi-Aventis). The cytotoxic agent may comprise CEP-8983/CEP-9722 (prodrug, Cephalon). The cytotoxic agent may comprise MK-4877 (Merck). The cytotoxic agent may comprise temozolomide, platins, cyclophosphamide, N-Methyl-N′-Nitro-N-Nitrosoguanidine (MNNG), topoisomerase I poisons, topotecan, oxaliplatin, gemcitabine or carboplatin.

There is provided, according to a 2^{nd} aspect of the present invention, a method comprising exposing poly (ADP-ribose) polymerase to a nucleic acid comprising the sequence RNNWCAAA, as set out above, in which the poly ADP-ribosylation activity of poly (ADP-ribose) polymerase is reduced as a result of the exposure.

We provide, according to a 3^{rd} aspect of the present invention, a method of inhibiting hepatitis B virus (HBV) replication by, so reducing the activity of poly (ADP-ribose) polymerase in or of the cell.

As a 4^{th} aspect of the present invention, there is provided a method of reducing the ability of a cell to repair DNA damage, by so reducing the activity of poly (ADP-ribose) polymerase in or of the cell. The method may be carried out in the presence of a cytotoxic agent. The cytotoxic agent may be a cytotoxic agent set out above.

We provide, according to a 5^{th} aspect of the present invention, a method of killing a cell, by so reducing the activity of poly (ADP-ribose) polymerase in or of the cell. The cell may comprise a cancer cell. The cell may comprise a breast cancer cell. The cancer cell may comprise a BRCA1 and BRCA2-deficient cancer cell.

In a 7^{th} aspect of the present invention, there is provided a nucleic acid capable of specifically binding to poly (ADP-ribose) polymerase and reducing its poly ADP-ribosylation activity. The nucleic acid may comprise the sequence RNNWCAAA, in which R is G or A, N is independently T, C, G or A and W is T or A.

According to an 8^{th} aspect of the present invention, we provide an isolated complex comprising poly (ADP-ribose) polymerase bound to a nucleic acid. The nucleic acid may comprise the sequence RNNWCAAA, in which R is G or A, N is independently T, C, G or A and W is T or A, such as ACATCAAA or ACTTCAAA.

We provide, according to a 9^{th} aspect of the invention, a pharmaceutical composition comprising a nucleic acid set out above together with a pharmaceutically acceptable excipient, diluent or carrier.

There is provided, in accordance with a 10^{th} aspect of the present invention, a nucleic acid as set out above for use in a method of inhibiting hepatitis B virus (HBV) replication. We further provide such a nucleic acid for use in a method of enhancing the cytotoxicity of ionising radiation or a drug.

As an 11^{th} aspect of the invention, we provide a method of identifying a molecule the method comprising exposing poly (ADP-ribose) polymerase to a nucleic acid set out above in the presence of a candidate molecule and detecting a decrease in binding of nucleic acid to poly (ADP-ribose) polymerase compared to in the absence of the candidate molecule.

We provide, according to a 12^{th} aspect of the invention, there is provided a method of identifying a molecule, such as suitable for use in the treatment or prevention of hepatitis B or cancer, the method comprising performing a method as set out above in the 4^{th} aspect of the invention in the presence of a candidate molecule and detecting an increase in poly ADP-ribosylation activity of poly (ADP-ribose) polymerase compared to in the absence of the candidate molecule.

Such a molecule as identified may be suitable for use in the treatment or prevention of hepatitis B or cancer.

According to a 13^{th} aspect of the present invention, we provide a method of treating an individual suffering or suspected to be suffering from hepatitis B virus infection. The method may comprise administering a therapeutically effective amount of a nucleic acid set out above.

There is provided, according to a 14^{th} aspect of the present invention, a method of treating an individual suffering or suspected to be suffering from cancer. The method may comprise administering a therapeutically effective amount of a nucleic acid set out above. The method may further comprise administering a cytotoxic drug or exposure to ionising radiation.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1A and FIG. 1B show the identification of an important regulatory sequence in HBV core promoter.

FIG. 1A shows the effect of URR deletions relative to the luciferase expression of the wild-type (WT) HBV core promoter in HepG2. Deletions resulting in 50% increase or 75% decrease in luciferase expression are deemed functionally significant. The relative positions of factors known to bind these sites are indicated as white circles in the NRE and black circles in the enhancer II region. They are—(A) NREBP, (B) FTF, (C) C/EBP, (D) HNF3, (E) HNF1 and (F) SP-1. [B] Deduction of the exact nucleotides required for the novel factor to exert its effects. The HBV core promoter sequence is as indicated, and the relative luciferase expression for each deletion (Del) is indicated as a percentage of wild-type.

FIG. 2A, FIG. 2B, FIG. 2C and FIG. 2D show that PARP-1 binds the HBV core promoter.

FIG. 2A shows that a biotinylated probe containing the “ACTTCAAA” sequence binds a novel factor in a dose-dependent manner to produce a band that cannot be found with the EBNA negative control probe.

FIG. 2B shows that streptavidin pull-down assays bound the novel factor but not the EBNA probe. 20 μl of the flow-through was run alongside the samples, and shows that the novel factor was enriched with the biotinylated probes. The novel factor sent for MALDI-TOF/TOF analysis.

FIG. 2C shows that a MALDI-TOF/TOF analysis of the novel factor reveals the only candidate to be PARP-1. Sequenced peptides are underlined and bold.

FIG. 2D shows an EMSA showing the specificity of PARP-1 for the “ACTTCAAA” sequence. Small excess of specific probe removed the PARP-1 specific band while large excess of poly-dIdC failed to do so. The PARP-1 specific antibody successfully competed with biotinylated probe hence diminished the specific band, whereas the non-specific HNF4α antibody was unable to do so.

FIG. 3A, FIG. 3B and FIG. 3C shows that PARP-1 knock-down decreases HBV replication.

FIG. 3A shows a HBV-RFP construct used to produce HBV. Viral production is driven by its own promoter.

FIG. 3B shows that PARP-1 specific knock-down rapidly decreases PARP-1 protein expression even at 24 h, during which the HBV-RFP construct was introduced into HepG2.

FIG. 3C shows that a PARP-1 specific knock-down reduces cccDNA synthesis from pgRNA. ***p<0.001.

FIG. 4A, FIG. 4B, FIG. 4C and FIG. 4D show that PARP-1 inhibition enhances transcription at the HBV core promoter.

FIG. 4A shows the effect of PARP inhibition with 3-aminobenzamide (3-AB) on PARP-1 motif dependent transcription. HepG2 and Huh-7 cells treated with 3-AB have increased luciferase expression when transfected with the wild-type HBV core promoter (WT). This effect is specific for PARP-1 as 3-AB had negligible effect on the mutant HBV core promoter with deletion of the PARP-1 binding site (Del-PARP1 motif). ***p<0.001

FIG. 4B shows PARP-1 inhibition with 15 μM PJ-34 increases the amount of HBV transcripts in HepG2 cells. Cells transfected with HBV-RFP remained viable with PJ-34 treatment 96 hours post-transfection. ***p<0.001.

FIG. 4C shows that PARP-1 binds the HBV core promoter in the vicinity of SP-1 and hnRNP K.

FIG. 4D shows that the PARP-1 enzyme ADP-ribosylates multiple transcription factors, preventing their binding to DNA. PARP inhibitors therefore enhance pgRNA synthesis hence HBV replication. Abbreviations: R-ADP-ribose; P-posphorylation.

FIG. 5A and FIG. 5B show the PARP-1 recognition motif.

FIG. 5A shows the effect of single base mutations on each of the nucleotides within the HBV “ACTTCAAA” PARP-1 binding sequence on PARP-1 dependent transcription. 50% increase or 75% decrease in wild-type activity are considered functionally significant.

FIG. 5B shows that the HBV PARP-1 binding sequence is highly similar to sequences of other PARP-1 binding promoters. Base frequency plot from multiple alignments is performed with the WebLogo program.

FIG. 6A, FIG. 6B and FIG. 6C show PARP-1 inhibition by motif recognition acts via domains other than the catalytic domain.

FIG. 6A shows PARP-1 inhibition by motif recognition. The sequences of DNA duplexes added are as indicated in grey. The PARP-1 motif is underlined. Mutations are indicated in black. Abbreviations: Non-sp K/D-Nuclear lysate from non-specific siRNA knock-down; PARP-1 K/D-Nuclear lysate from HepG2 treated with PARP-1 specific siRNA. *p<0.05; **p<0.01.

FIG. 6B shows the functional domains of human PARP-1. The N- and C-termini are marked by “N” and “C” respectively. Abbreviations: ZF1-Zinc finger 1; ZF2-Zinc Finger 2; ZF3-Zinc finger 3; BRCT-BRCA1 carboxy-terminal domain; WGR-WGR domain; Cat-Catalytic domain.

FIG. 6C shows the effect of over-expressing PARP-1 mutants bearing single amino acid substitutions to alanine on transcription in HepG2 cells. Amino acid K893 was mutated to isoleucine. Functional mutations are marked with “−” and result in the loss of luciferase expression when compared to over-expression of wild-type (WT) PARP-1. Mutations with little effect on motif recognition are indicated with “+”.

FIG. 7A, FIG. 7B, FIG. 7C and FIG. 7D show that PARP-1 inhibition by motif recognition enhances DNA damage-induced cell death.

FIG. 7A shows impaired DNA damage repair in HepG2 transfected with 3 tandem repeats of the PARP-1 motif. ***p<0.001

FIG. 7B shows increased apoptosis shortly after the induction of DNA damage in HepG2 transfected with the PARP-1 motif. **p<0.001

FIG. 7C shows increased cytotoxicity from DNA damage inducers in cells transfected with the PARP-1 motif. Dying cells stain positive for annexin V hence fluoresce green.

FIG. 7D shows reduced viability in Huh-7 cells transfected with 1 μg of the PARP-1 motif compared to cells inhibited with 10 μM clinical PARP inhibitors. DNA damage was induced 24 hours after PARP inhibition with 100 nM etoposide or 0.02% NMU, and the viable cells that remained 54 hours after PARP inhibition was observed by light microscopy.

FIG. 8A, FIG. 8B, FIG. 8C and FIG. 8D show that HBV replication impairs PARP-1 dependent DNA repair.

FIG. 8A shows that HBV replication indicated by cccDNA increase peaks at around 96 h post-transfection.

FIG. 8B shows that the HBV PARP-1 sequence (underlined) can inhibit PARP-1 enzymatic activity.

FIG. 8C shows that HBV replication impairs DNA damage repair as significantly greater proportion of cells have increased DNA damage when treated with DNA damage inducers.

FIG. 8D shows increased cytotoxicity from DNA damage inducers in cells transfected with HBV. Viable transfected cells fluoresce red due to RFP expression while dying cells stain positive for annexin V hence fluoresce green. HBV replication sensitizes cells to DNA damage induced apoptosis as reflected by the increased caspase activity in cells transfected with HBV-RFP. ***p<0.001.

FIG. 9A, FIG. 9B and FIG. 9C show that the over-expression of wild-type PARP-1 alone is sufficient to alleviate PARP-1 motif-dependent sensitization of cells to DNA damage inducers.

FIG. 9A shows that wild-type PARP-1 is over-expressed in the nucleus of HepG2 cells 48 hours after transfection. Abbreviation: O/E-over-expression.

FIG. 9B shows that the over-expression of wild-type PARP-1 in Huh-7 cells reduces DNA damage in cells transfected with the PARP-1 motif and treated with 0.01% NMU. ***p<0.001

FIG. 9C shows that the over-expression of wild-type PARP-1 reduces PARP-1 motif-sensitized apoptosis as indicated by a reduction in caspase activity in HepG2 cells treated with various DNA damage inducers. ***p<0.001

FIG. 10A and FIG. 10B show that the PARP-1 motif inhibits PARP-1 function in a sequence dependent manner.

FIG. 10A shows reduced impairment of DNA damage repair by bleomycin (20 ng/ml) when HepG2 cells are transfected with 3 tandem repeats of the mutant PARP-1 motif “ACAGGCCA”. ***p<0.001.

FIG. 10B shows alleviation of DNA damage induced apoptosis in HepG2 cells transfected with the mutant PARP-1 motif “ACAGGCCA” and treated with bleomycin (20 ng/ml). **p<0.01.

FIG. 11A and FIG. 11B show that treatment of HBV-transfected cells with the PARP-1 motif reduces HBV replication.

FIG. 11A shows co-transfection of 500 ng PARP-1 motif with 1 μg of HBV-RFP reduces the amount of HBV transcripts in HepG2 cells. The relative number of HBV-RFP transfected cells that remain 96 hours after co-transfection with the PARP-1 motif is also reduced. **p<0.01.

FIG. 11B shows the loss of HBs expression by immunofluorescence staining in HepG2 cells co-transfected with 500 ng PARP-1 motif and 1 μg HBV-RFP. The effect of PARP-1 specific knock-down by PARP-1 specific siRNA on HBs expression is also shown for comparison.

### DETAILED DESCRIPTION

We describe a molecule capable of binding to and inhibiting the activity of poly (ADP-ribose) polymerase (PARP-1). We refer to this molecule generally as a “PARP-1 binding motif”.

The PARP-1 binding motif may comprise a nucleic acid comprising the sequence RNNWCAAA, in which R is G or A, N is independently T, C, G or A and W is T or A. We find that such a nucleic acid is capable of binding to and inhibiting the activity of poly (ADP-ribose) polymerase. Such a nucleic acid, together with its fragments, homologues, variants and derivatives is disclosed in further detail below.

The Examples demonstrate that the PARP-1 binding motif described here is capable of reducing or inhibiting the poly ADP-ribosylation activity of poly (ADP-ribose) polymerase. We therefore describe a method of reducing or inhibiting the activity of poly (ADP-ribose) polymerase, the method comprising exposing poly (ADP-ribose) polymerase to a PARP-1 binding motif nucleic acid.

The PARP-1 binding motif nucleic acid may be used for various means, including inhibiting the replication of hepatitis B virus (HBV) replication or in order to kill a cancer cell.

PARP-1 Binding Motif molecules may be used for a variety of means, for example, administration to an individual suffering from, or suspected to be suffering from, cancer such as breast cancer, for the treatment thereof. They may also be used for production or screening of anti-cancer or anti-hepatitis agents, or for production or screening of agents capable of killing cells.

We therefore provide for the use of PARP-1 Binding Motif in screening for drugs against cancer, for example breast cancer and for treating, preventing or alleviating hepatitis, for example hepatitis B. The cancer may comprise invasive breast cancer.

These are described in further detail below.

### PARP-1 Binding Motif

The methods and compositions described here may employ, as a means for inhibiting or reducing poly (ADP-ribose) polymerase activity, PARP-1 Binding Motifs, including PARP-1 Binding Motif polynucleotides, PARP-1 Binding Motif nucleotides and PARP-1 Binding Motif nucleic acids, as well as variants, homologues, derivatives and fragments of any of these.

In addition, we disclose particular PARP-1 Binding Motif fragments useful for the methods of treatment and diagnosis described here.

The PARP-1 Binding Motif nucleic acids may also be used for the methods of treatment or prophylaxis described.

RNNWCAAA PARP-1 Binding Motif

As the term is used in this document, a PARP-1 Binding Motif is a nucleic acid that comprises, preferably consists of, the sequence RNNWCAAA, where R independently is G or A, N is independently T, C, G or A and W is independently T or A.

Examples of PARP-1 Binding Motifs include the sequences set out in Table D1 below.

TABLE D1
PARP-1 Binding Motif Sequences RNNWCAAA

| | | | | | |
|---|---|---|---|---|---|
| GTTTCAAA | GTTACAAA | GTCTCAAA | GTCACAAA | GTGTCAAA | GTGACAAA |
| GTATCAAA | GTAACAAA | GCTTCAAA | GCTACAAA | GCCTCAAA | GCCACAAA |
| GCGTCAAA | GCGACAAA | GCATCAAA | GCAACAAA | GGTTCAAA | GGTACAAA |
| GGCTCAAA | GGCACAAA | GGGTCAAA | GGGACAAA | GGATCAAA | GGAACAAA |
| GATTCAAA | GATACAAA | GACTCAAA | GACACAAA | GAGTCAAA | GAGACAAA |
| GAATCAAA | GAAACAAA | ATTTCAAA | ATTACAAA | ATCTCAAA | ATCACAAA |
| ATGTCAAA | ATGACAAA | ATATCAAA | ATAACAAA | ACTTCAAA | ACTACAAA |
| ACCTCAAA | ACCACAAA | ACGTCAAA | ACGACAAA | ACATCAAA | ACAACAAA |
| AGTTCAAA | AGTACAAA | AGCTCAAA | AGCACAAA | AGGTCAAA | AGGACAAA |
| AGATCAAA | AGAACAAA | AATTCAAA | AATACAAA | AACTCAAA | AACACAAA |
| AAGTCAAA | AAGACAAA | AAATCAAA | AAAACAAA | | |

For example, a PARP-1 Binding Motif may comprise the sequence ACTTCAAA or it may comprise the sequence ACATCAAA.

ANWNCAAA PARP-1 Binding Motif

A PARP-1 Binding Motif may also comprise the sequence ANWNCAAA, where N is independently T, C, G or A and W is independently T or A. Examples of ANWNCAAA PARP-1 Binding Motif sequences are set out in Table D2 below.

TABLE D2
ANWNCAAA

| | | | | | |
|---|---|---|---|---|---|
| ATTTCAAA | ATTCCAAA | ATTGCAAA | ATTACAAA | ATATCAAA | ATACCAAA |
| ATAGCAAA | ATAACAAA | ACTTCAAA | ACTCCAAA | ACTGCAAA | ACTACAAA |
| ACATCAAA | ACACCAAA | ACAGCAAA | ACAACAAA | AGTTCAAA | AGTCCAAA |
| AGTGCAAA | AGTACAAA | AGATCAAA | AGACCAAA | AGAGCAAA | AGAACAAA |
| AATTCAAA | AATCCAAA | AATGCAAA | AATACAAA | AAATCAAA | AAACCAAA |
| AAAGCAAA | AAAACAAA | | | | |

ANTNCAAA PARP-1 Binding Motif

A PARP-1 Binding Motif may also comprise the sequence ANTNCAAA, where N is independently T, C, G or A. Examples of ANTNCAAA PARP-1 Binding Motif sequences are set out in Table D2 below.

TABLE D3
ANTNCAAA

| | | | | | |
|---|---|---|---|---|---|
| ATTTCAAA | ATTCCAAA | ATTGCAAA | ATTACAAA | ACTTCAAA | ACTCCAAA |
| ACTGCAAA | ACTACAAA | AGTTCAAA | AGTCCAAA | AGTGCAAA | AGTACAAA |
| AATTCAAA | AATCCAAA | AATGCAAA | AATACAAA | | |

The terms “PARP-1 Binding Motif polynucleotide”, “PARP-1 Binding Motif nucleotide” and “PARP-1 Binding Motif nucleic acid” may be used interchangeably, and should be understood to specifically include both DNA and RNA PARP-1 Binding Motif sequences, whether single stranded or double stranded (as described in further detail below).

PARP-1 Binding Motif nucleic acids may be used for a variety of means, for example, administration to an individual suffering from, or suspected to be suffering from a cancer such as breast cancer, for the treatment thereof. PARP-1 Binding Motif nucleic acids may also be administered to an individual suffering from, or suspected to be suffering from, hepatitis B (HBV) infection.

“Polynucleotide” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

The term “nucleotide sequence” may means DNA.

Other Nucleic Acids

We also provide nucleic acids which are fragments, homologues, variants or derivatives of PARP-1 Binding Motif nucleic acids. The terms “variant”, “homologue”, “derivative” or “fragment” in relation to PARP-1 Binding Motif nucleic acid include any substitution of, variation of, modification of, replacement of, deletion of or addition of one (or more) nucleic acids from or to the sequence of a PARP-1 Binding Motif nucleotide sequence. Unless the context admits otherwise, references to “PARP-1 Binding Motif” and include references to such variants, homologues, derivatives and fragments of PARP-1 Binding Motif.

The resultant nucleotide sequence may comprise PARP-1 Binding Motif activity. The term “homologue” may be intended to cover identity with respect to structure and/or function such that the resultant nucleotide sequence has PARP-1 Binding Motif activity. For example, a homologue etc of PARP-1 Binding Motif may be capable of binding to poly (ADP-Ribose) polymerase. A homologue etc of PARP-1 Binding Motif may be capable of inhibiting or reducing the activity of poly (ADP-Ribose) polymerase. Methods of assaying binding to enzymes such as poly (ADP-Ribose) polymerase as well as methods of assaying enzyme activity, e.g., to detect changes in activity such as enzyme inhibition, are well known in the art.

The homologue, variant, derivative or fragment of PARP-1 Binding Motif may comprise a nucleic acid sequence with sequence identity or similarity to the sequences described here. With respect to sequence identity (i.e. similarity), there may be at least 70%, at least 75%, at least 85% or at least 90% sequence identity. There may be at least 95%, such as at least 98%, sequence identity to a relevant sequence (e.g., a PARP-1 Binding Motif sequence RNNWCAAA ANWNCAAA or ANTNCAAA, or as shown above in Table D1, Table D2 or Table D3). These terms also encompass allelic variations of the sequences.

Variants, Derivatives and Homologues

PARP-1 Binding Motif nucleic acid variants, fragments, derivatives and homologues may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of this document, it is to be understood that the polynucleotides may be modified by any method available in the art. Such modifications may be carried out in order to enhance the in vivo activity or life span of polynucleotides of interest.

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

The terms “variant”, “homologue” or “derivative” in relation to a nucleotide sequence include any substitution of, variation of, modification of, replacement of, deletion of or addition of one (or more) nucleic acid from or to the sequence. Said variant, homologues or derivatives may code for a polypeptide having biological activity. Such fragments, homologues, variants and derivatives of PARP-1 Binding Motif may comprise modulated activity, as set out above.

As indicated above, with respect to sequence identity, a “homologue” may have at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence (e.g., a PARP-1 Binding Motif sequence RNNWCAAA ANWNCAAA or ANTNCAAA, or as shown above in Table D1, Table D2 or Table D3).

There may be at least 95% identity, at least 96% identity, at least 97% identity, at least 98% identity or at least 99% identity. Nucleotide identity comparisons may be conducted as described above. A sequence comparison program which may be used is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

Hybridisation

We further describe nucleotide sequences that are capable of hybridising selectively to any of the sequences presented herein, or any variant, fragment or derivative thereof, or to the complement of any of the above. Nucleotide sequences may be at least 15 nucleotides in length, such as at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, may be at least 40% homologous, at least 45% homologous, at least 50% homologous, at least 55% homologous, at least 60% homologous, at least 65% homologous, at least 70% homologous, at least 75% homologous, at least 80% homologous, at least 85% homologous, at least 90% homologous, or at least 95% homologous to the corresponding nucleotide sequences presented herein (e.g., a PARP-1 Binding Motif sequence RNNWCAAA ANWNCAAA or ANTNCAAA, or as shown above in Table D1, Table D2 or Table D3). Such polynucleotides may be generally at least 70%, at least 80 or 90% or at least 95% or 98% homologous to the corresponding nucleotide sequences over a region of at least 20, such as at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, such as less than 100 fold as intense as the specific interaction observed with the target DNA. The intensity of interaction may be measured, for example, by radiolabelling the probe, e.g. with ³²P or ³³P or with non-radioactive probes (e.g., fluorescent dyes, biotin or digoxigenin).

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

We provide nucleotide sequences that may be able to hybridise to the PARP-1 Binding Motif nucleic acids, fragments, variants, homologues or derivatives under stringent conditions (e.g. 65° C. and 0.1×SSC (1×SSC=0.15 M NaCl, 0.015 M Na₃ Citrate pH 7.0)).

Generation of Homologues, Variants and Derivatives

Polynucleotides which are not 100% identical to the relevant sequences (e.g., a PARP-1 Binding Motif sequence RNNWCAAA ANWNCAAA or ANTNCAAA, or as shown above in Table D1, Table D2 or Table D3) but which are also included, as well as homologues, variants and derivatives of PARP-1 Binding Motif can be obtained in a number of ways. Other variants of the sequences may be obtained for example by probing DNA libraries made from a range of individuals, for example individuals from different populations. For example, PARP-1 Binding Motif homologues may be identified from other individuals, or other species. Further recombinant PARP-1 Binding Motif nucleic acids and polypeptides may be produced by identifying corresponding positions in the homologues, and synthesising or producing the molecule as described elsewhere in this document.

In addition, other viral/bacterial, or cellular homologues of PARP-1 Binding Motif, particularly cellular homologues found in mammalian cells (e.g. rat, mouse, bovine and primate cells), may be obtained and such homologues and fragments thereof in general will be capable of selectively hybridising to human PARP-1 Binding Motif. Such homologues may be used to design non-human PARP-1 Binding Motif nucleic acids, fragments, variants and homologues. Mutagenesis may be carried out by means known in the art to produce further variety.

Sequences of PARP-1 Binding Motif homologues may be obtained by probing cDNA libraries made from or genomic DNA libraries from other animal species, and probing such libraries with probes comprising all or part of any of the PARP-1 Binding Motif nucleic acids, fragments, variants and homologues, or other fragments of PARP-1 Binding Motif under conditions of medium to high stringency.

Similar considerations apply to obtaining species homologues and allelic variants of the polypeptide or nucleotide sequences disclosed here.

Variants and strain/species homologues may also be obtained using degenerate PCR which will use primers designed to target sequences within the variants and homologues encoding conserved amino acid sequences within the sequences of the PARP-1 Binding Motif nucleic acids. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments the PARP-1 Binding Motif sequences.

In addition, homologous sequences may be identified by searching nucleotide and/or protein databases using search algorithms such as the BLAST suite of programs.

Alternatively, such polynucleotides may be obtained by site directed mutagenesis of characterised sequences, for example, PARP-1 Binding Motif nucleic acids, or variants, homologues, derivatives or fragments thereof. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides described here may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 8, 9, 10, or 15, such as at least 20, for example at least 25, 30 or 40 nucleotides in length, and are also encompassed by the term “polynucleotides” as used herein.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Primers comprising fragments of PARP-1 Binding Motif are particularly useful in the methods of detection of poly (ADP-Ribose) polymerase expression, such as down-regulation of poly (ADP-Ribose) polymerase activity. Suitable primers for amplification of PARP-1 Binding Motif may be generated from any suitable stretch of PARP-1 Binding Motif. Primers which may be used include those capable of amplifying a sequence of PARP-1 Binding Motif which is specific, i.e., does not have significant homology to YAP for example.

Although PARP-1 Binding Motif primers may be provided on their own, they are most usefully provided as primer pairs, comprising a forward primer and a reverse primer.

Longer polynucleotides will generally be produced using recombinant means, for example using a PCR (polymerase chain reaction) cloning techniques. This will involve making a pair of primers (e.g. of about 15 to 30 nucleotides), bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

Polynucleotides or primers may carry a revealing label. Suitable labels include radioisotopes such as ³²P or ³⁵S, digoxigenin, fluorescent dyes, enzyme labels, or other protein labels such as biotin. Such labels may be added to polynucleotides or primers and may be detected using by techniques known per se. Polynucleotides or primers or fragments thereof labelled or unlabeled may be used by a person skilled in the art in nucleic acid-based tests for detecting or sequencing polynucleotides in the human or animal body.

Such tests for detecting generally comprise bringing a biological sample containing DNA or RNA into contact with a probe comprising a polynucleotide or primer under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO89/03891 and WO90/13667.

Tests for sequencing nucleotides, for example, the PARP-1 Binding Motif nucleic acids, involve bringing a biological sample containing target DNA or RNA into contact with a probe comprising a polynucleotide or primer under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Small Molecule PARP-1 Inhibitors

We describe small molecule inhibitors of PARP-1 activity. Included are any molecules that resemble or have a biological activity of the PARP-1 binding motif (e.g., a PARP-1 Binding Motif sequence RNNWCAAA ANWNCAAA or ANTNCAAA, or a homologue, derivative, fragment or variant thereof). We refer to these as “small molecule PARP-1 binding motifs”.

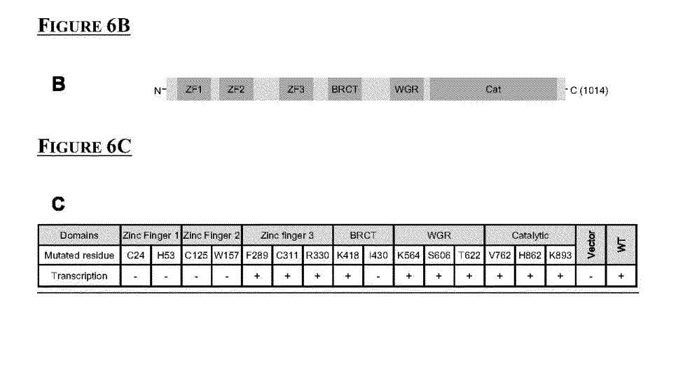

Such small molecule PARP-1 Binding Motifs may bind the PARP-1 domain(s) which interacts with the PARP-1 binding motif. As shown in FIG. 6C, the domains involved in binding the PARP-1 binding motif are likely zinc fingers 1 and 2, although the possibility that other domains are involved (such as the BRCT domain) cannot be excluded.

In order to design such small molecule PARP-1 binding motifs, the crystal structure of the PARP-1 binding motif-PARP-1 domain complex is resolved. Residues within these domain(s) that form the binding groove for the PARP-1 binding motif are then mapped.

Molecules with properties (shape, size, charge, hydrophobicity and hydrophilicity etc) that complement the properties of the binding groove may be tested for their ability to inhibit PARP-1 in the manner that the PARP-1 binding motif does.

An example of assay to test if these molecules have biological function(s) similar to the PARP-1 binding motif is the luciferase assay to test the ability of PARP-1 to bind the PARP-1 binding motif within the HBV core promoter. Pre-treatment of cells with the molecule will result in reduced luciferase expression.

To demonstrate that the molecule can inhibit PARP-1 enzymatic activity, any suitable assay, such as the histone modification assay may be used. The biological consequence of PARP-1 inhibition with the molecule can then be tested with assays such as the Comet assay.

### Poly (ADP-Ribose) Polymerase (PARP-1)

The methods and compositions described here make use of PARP-1 polypeptides, which are described in detail below. As used here, the term “PARP-1” is intended to refer to a sequence set out in Table D4 below.

TABLE D4
Poly (ADP-Ribose) Polymerase Polypeptides

| GenBank Accession | | |
|---|---|---|
| Number | Name | Species |
| NP_031441.2 | poly [ADP-ribose] polymerase 1 | M. musculus |
| NP_001609.2 | poly [ADP-ribose] polymerase 1 | H. sapiens |
| NP_001081571.1 | poly [ADP-ribose] polymerase 1 | X. laevis |
| NP_001038407.1 | poly [ADP-ribose] polymerase 1 | D. rerio |
| NP_001104452.1 | Poly-(ADP-ribose) polymerase | D. melanogaster |
| NP_850165.1 | PARP2 (POLY(ADP-RIBOSE) | A. thaliana |
| | POLYMERASE 2); DNA | |
| | binding/NAD or NADH | |
| | binding/NAD + ADP- | |
| | ribosyltransferase/zinc | |
| | ion binding | |
| XP_960982.2 | hypothetical protein NCU08852 | N. crassa |
| NP_037195.1 | poly [ADP-ribose] polymerase 1 | R. norvegicus |
| XP_001090984.1 | PREDICTED: poly [ADP- | M. mulatta |
| | ribose] polymerase 1 isoform 4 | |

PARP-1 is an important player in DNA damage repair [47-51]. This is demonstrated by the increased sensitivity to genotoxic stress in PARP-1 knock-out mice [23, 49]. Incidence of hepatocellular carcinoma (HCC) in PARP-1 knock-out mice heterozygous for Ku80 is also increased, suggesting the importance of PARP-1 in the liver [23, 52]. PARP-1 dependent DNA damage repair requires the active PARP-1 enzyme [47, 51, 53, 54]. This requires PARP-1 binding to DNA strand breaks to activate its catalytic activity, resulting in auto-ADP-ribosylation [55]. The extensive ADP-ribose polymers attract ATM or ATR and recruits DNA repair machinery, thus PARP-1 is an important sensor of DNA damage [44, 49, 50]. The involvement of PARP-1 in multiple DNA repair pathways is being exploited in cancer therapy, especially in cells that are BRCA-deficient where DNA repair by homologous recombination cannot take place [24, 33-37, 53], where the inhibition of its enzymatic activity prevents DNA single-strand break, double-strand break and base-excision repair to enhance cytotoxicity in cancer cells induced by irreparable DNA damage as intended [33, 34, 37].

Besides being involved in DNA damage repair, the multi-functional PARP-1 has been shown in microarray analyses to be important for the transcriptional regulation of many genes [56, 57]. By recruiting topoisomerase-IIβ to cleave and unwind DNA, it brings relief to chromatin structures hence enables transcription [58]. PARP-1 binding to promoters has also been found to exclude histone H1 for active transcription [59]. As an activated enzyme, PARP-1 adds ADP-ribose moieties to itself and many nuclear proteins such as the transcription factors NFκB and p53 as well as histones [60-65], altering their activity by conferring an increase in net negative charge [39-42, 46]. Furthermore, it can act as a transcriptional activator or repressor by binding DNA in a sequence-specific manner, directly regulating the expression of viral transcripts and genes including inflammation modulators such as IFNγ [29, 66-70]. The extent of PARP-1 sequence-specific transcriptional regulation however remains elucidated.

Many questions regarding PARP-1 sequence-specific transcriptional regulation remain to be answered. A PARP-1 sequence-specific binding motif has yet to be clearly defined. The question of how PARP-1 discriminates between non-sequence specific DNA strand break binding and sequence-specific binding, as well as how their respective binding affect its catalytic activity remain to be resolved.

The following text is adapted from OMIM entry 173870MGI.

Poly (ADP-Ribose) polymerase is also known as PARP-1, PPOL, poly (ADP-Ribose) synthetase, ADP-ribosyl transferase, ADPRT and ADPRT1. It has gene map locus: 1q42

Description:

The chromatin-associated enzyme poly(ADP-ribose) polymerase (ADPRT; EC 2.4.2.30) uses NAD as substrate to catalyze both the covalent transfer of ADP-ribose to a variety of nuclear protein acceptors and subsequently the transfer of an additional 60 to 80 ADP-ribose units to the initial moiety. Nuclear proteins that become predominantly poly(ADP-ribosyl)ated include nucleosomal core histones, histone H1 (see 142711), HMG proteins (see 163910), and topoisomerases I (126420) and II (see 126430). ADP ribosyltransferase is required for cellular repair. Inhibitors of this enzyme potentiate the lethal effects of noxious agents. During repair, NAD+ is consumed and the NAD+ content of the cell decreases. Concomitantly, nuclear proteins are ADP-ribosylated. The enzyme is induced by single-strand breaks in DNA which serve as cosubstrate for the reaction.

Cloning

Alkhatib et al. (1987) isolated cDNA clones for this enzyme from a human hepatoma lambda library and studied its expression. Using synthetic oligonucleotide probes based on the partial amino acid sequence of poly(ADP-ribose), Kurosaki et al. (1987) isolated and sequenced cDNA clones for the enzyme. The open reading frame encodes a protein of 1,013 amino acid residues with a molecular mass of 113,203 Da.

Gene Function

Loetscher et al. (1987) proposed that poly(ADP-ribose) may signal altered metabolic conditions to the chromatin. They were led to this proposal from the finding that the constitutive level of posttranslational poly(ADP-ribose) modification of chromatin proteins in mammalian cells is related to the availability of NAD, which varies in different physiologic and pathologic states.

ADP-ribosylation is a eukaryotic posttranslational modification of proteins that is strongly induced by the presence of DNA strand breaks and plays a role in DNA repair and the recovery of cells from DNA damage. Grube and Burkle (1992) found a strong positive correlation (r=0.84; P much less than 0.001) between poly(ADP-ribose) polymerase, or PARP, activity and life span, with human cells displaying, for example, about 5 times the activity of rat cells. The cells studied were mononuclear leukocytes. Grube and Burkle (1992) suggested that higher poly(ADP-ribosyl)ation capacity may contribute to the efficient maintenance of genome integrity.

Yu et al. (2002) demonstrated that PARP1 activation is required for translocation of apoptosis-inducing factor (AIF; 300169) from the mitochondria to the nucleus and that AIF is necessary for PARP1-dependent cell death. N-methyl-N-prime-nitro-N-nitrosoguanidine, hydrogen peroxide, and NMDA induce AIF translocation and cell death, which is prevented by PARP inhibitors or genetic knockout of PARP1, but is caspase independent. Microinjection of an antibody to AIF protects against PARP1-dependent cytotoxicity. Yu et al. (2002) concluded that their data support a model in which PARP1 activation signals AIF release from mitochondria, resulting in a caspase-independent pathway of programmed cell death.

Using cultured human cells from malignant cell lines, Nicholson et al. (1995) demonstrated that PARP is proteolytically cleaved at the onset of apoptosis by caspase-3 (CASP3; 600636), which they called apopain. In addition, they showed that inhibition of apopain-mediated PARP cleavage attenuates apoptosis in vitro.

Tomoda et al. (1991) found that in contrast to reactive proliferative diseases, malignant lymphomas showed increased expression of poly(ADP-ribose) synthetase as demonstrated by level of mRNA.

Cohen-Armon et al. (2004) found that poly(ADP-ribose) polymerase-1 is activated in neurons that mediate several forms of long-term memory in Aplysia. Because poly(ADP-ribosyl)ation of nuclear proteins is a response to DNA damage in virtually all eukaryotic cells, it is surprising that activation of the polymerase occurs during learning and is required for long-term memory. Cohen-Armon et al. (2004) suggested that the fast and transient decondensation of chromatin structure by poly(ADP-ribosyl)ation enables the transcription needed to form long-term memory without strand breaks in DNA.

Vasquez et al. (2001) included the PARP gene in their list of candidate genes for enhancing gene targeting. Gene targeting by homologous recombination, which was developed by Smithies et al. (1985), Thomas et al. (1986), and Thomas and Capecchi (1987), has proven highly valuable in studies of gene structure and function and offers a potential tool for gene-therapeutic applications. A limitation constraining this technology is the low rate of homologous recombination in mammalian cells and the high rate of random (nontargeted) integration of the vector DNA. Vasquez et al. (2001) considered possible ways to overcome these limitations in the framework of the current understanding of recombination mechanisms and machinery. Several studies suggested that transient alteration of the levels of recombination proteins, by overexpression or interference with expression, may be able to increase homologous recombination or decrease random integration.

Several PARPs localize to the spindle in vertebrate cells, suggesting that PARPs and/or poly(ADP-ribose) (PAR) have a role in spindle function. Chang et al. (2004) showed that PAR is enriched in the spindle and is required for spindle function. PAR hydrolysis or perturbation led to rapid disruption of spindle structure, and hydrolysis during spindle assembly blocked the formation of bipolar spindles. PAR exhibited localization dynamics that differed from known spindle proteins and were consistent with a low rate of turnover in the spindle. Thus, Chang et al. (2004) concluded that PAR is a nonproteinaceous, nonchromosomal component of the spindle required for bipolar spindle assembly and function.

Kim et al. (2004) described nucleosome binding properties of PARP1 that promoted the formation of compact, transcriptionally repressed chromatin structures. PARP1 bound in a specific manner to nucleosomes and modulated chromatin structure through NAD(+)-dependent automodification, without modifying core histones or promoting the disassembly of nucleosomes. The automodification activity of PARP1 was potently stimulated by nucleosomes, causing the release of PARP1 from chromatin. The NAD(+)-dependent activities of PARP1 were reversed by poly(ADP-ribose) glycohydrolase (PARG; 603501) and were inhibited by ATP. In vivo, PARP1 incorporation was associated with transcriptionally repressed chromatin domains that were spatially distinct from both histone H1-repressed domains and actively transcribed regions. Kim et al. (2004) concluded that PARP1 functions both as a structural component of chromatin and as a modulator of chromatin structure through its intrinsic enzymatic activity.

Pavri et al. (2005) determined that PARP1 was necessary for retinoic acid (RA)-dependent transcription in HeLa cells, and transcription required the direct interaction of PARP1 with the mediator complex (see MED6; 602984). The interaction did not require the C-terminal catalytic domain of PARP1. By chromatin immunoprecipitation of a mouse embryonic carcinoma cell line, Pavri et al. (2005) found that Parp1 localized to the RA-responsive promoter of the mouse Rarb2 gene (RARB; 180220). Parp1 was necessary for the activation of the mediator complex and for transcription from the Rarb2 promoter. Pavri et al. (2005) also found that Parp1 functioned at a step prior to the association of TFIID (see 313650) and mediator with promoter sequences.

Bryant et al. (2005) showed that PARP inhibitors trigger gamma-H2AX (see 601772) and RAD51 (179617) foci formation. They proposed that, in the absence of PARP1, spontaneous single-strand breaks collapse replication forks and trigger homologous recombination for repair. Furthermore, Bryant et al. (2005) showed that BRCA2 (600185)-deficient cells, as a result of their deficiency in homologous recombination, are acutely sensitive to PARP inhibitors, presumably because resultant collapsed replication forks are no longer repaired. Thus, PARP1 activity is essential in homologous recombination-deficient BRCA2 mutant cells. Bryant et al. (2005) exploited this requirement in order to kill BRCA2-deficient tumors by PARP inhibition alone. Treatment with PARP inhibitors is likely to be highly tumor specific, because only the tumors (which are BRCA2-null) in BRCA2 heterozygous patients are defective in homologous recombination. Bryant et al. (2005) concluded that the use of an inhibitor of a DNA repair enzyme alone to selectively kill a tumor, in the absence of an exogenous DNA-damaging agent, represents a new concept in cancer treatment.

Farmer et al. (2005) showed that BRCA1 (113705) or BRCA2 dysfunction unexpectedly and profoundly sensitizes cells to the inhibition of PARP enzymatic activity, resulting in chromosomal instability, cell cycle arrest, and subsequent apoptosis. This seems to be because the inhibition of PARP leads to the persistence of DNA lesions normally repaired by homologous recombination. Farmer et al. (2005) concluded that their results illustrate how different pathways cooperate to repair damage, and suggest that the targeted inhibition of particular DNA repair pathways may allow the design of specific and less toxic therapies for cancer.

Andrabi et al. (2006) and Yu et al. (2006) demonstrated that the product of PARP1 activity, poly(ADP-ribose) (PAR) polymer, mediates PARP1-induced cell death. Andrabi et al. (2006) showed PAR polymer alone could induce cell death in primary mouse cortical neurons in a caspase- and Parp1-independent manner. Degradation of PAR polymer by PAR glycohydrolase (PARG; 603501) or phosphodiesterase-1 (see PDE1A, 171890) prevented PAR polymer-induced cell death in cultured neurons, and increased Parg expression in mice reduced damage caused by ischemia following middle cerebral artery occlusion. Yu et al. (2006) showed that PAR polymer was the cell death signal that induced the release of Aif from mitochondria mouse cortical neurons and induced its translocation to nuclei. They also showed that Parg prevented Parp1-dependent Aif release. Furthermore, cells with reduced levels of Aif were resistant to Parp1-dependent cell death and PAR polymer cytotoxicity.

Apoptosis controls the final numbers of neurons during brain development. Midorikawa et al. (2006) found that mouse Kif4 (300521), a microtubule-based molecular motor, regulated apoptosis of juvenile neurons by interacting directly with Parp1. The C-terminal domain of Kif4 suppressed Parp1 enzymatic activity. When neurons were stimulated by membrane depolarization, calcium signaling mediated by Camk2 (see 114078) induced dissociation of Kif4 from Parp1, resulting in upregulation of Parp1 activity, which supported neuron survival. After dissociation from Parp1, Kif4 entered the cytoplasm from the nucleus and moved to the distal part of neurites in a microtubule-dependent manner. Midorikawa et al. (2006) concluded that KIF4 controls the activity-dependent survival of postmitotic neurons by regulating PARP1 activity in brain development.

Krishnakumar et al. (2008) used genomic and gene-specific approaches to show that 2 factors, histone H1 and PARP1, exhibit a reciprocal pattern of chromatin binding at many RNA polymerase II-transcribed promoters. PARP1 was enriched and H1 was depleted at these promoters. This pattern of binding was associated with actively transcribed genes. Furthermore, Krishnakumar et al. (2008) showed that PARP1 acts to exclude H1 from a subset of PARP1-stimulated promoters, suggesting a functional interplay between PARP1 and H1 at the level of nucleosome binding. Thus, Krishnakumar et al. (2008) concluded that although H1 and PARP1 have similar nucleosome-binding properties and effects on chromatin structure in vitro, they have distinct roles in determining gene expression in vivo.

‘Synthetic lethality’ as a treatment for cancer refers to an event in which tumor cell death results from lethal synergy of 2 otherwise nonlethal events. Fong et al. (2009) used this model to treat breast cancer cells that have homozygous loss of the tumor suppressor genes BRCA1 (113705) or BRCA2 (600185) with a PARP inhibitor, resulting in the induction of selective tumor cytotoxicity and the sparing of normal cells. The method aims at inhibiting PARP-mediated single-strand DNA repair in cells with deficient homologous-recombination double-strand DNA repair, which leads to unrepaired DNA breaks, the accumulation of DNA defects, and cell death. Heterozygous BRCA mutant cells retain homologous-recombination function and are not affected by PARP inhibition. In vitro, BRCA1-deficient and BRCA2-deficient cells were up to 1,000-fold more sensitive to PARP inhibition than wildtype cells, and tumor growth inhibition was also demonstrated in BRCA2-deficient xenografts. Fong et al. (2009) reported a phase 1 clinical trial of an orally active PARP inhibitor olaparib (AZD2281 or KU-0059436) in 60 patients with mainly breast or ovarian cancer (612555; 604370), including 22 BRCA mutation carriers and 1 who was likely a mutation carrier but declined genetic testing. Durable objective antitumor activity was observed only in confirmed carriers of a BRCA1 or BRCA2 mutation; no objective antitumor responses were observed in patients without known BRCA mutations. Twelve (63%) of 19 BRCA carriers with ovarian, breast, or prostate cancers showed a clinical benefit from treatment with olaparib, with radiologic or tumor-marker responses or meaningful disease stabilization. The drug had an acceptable side-effect profile and did not have the toxic effects commonly associated with conventional chemotherapy. Fong et al. (2009) concluded that PARP inhibition has antitumor activity in BRCA mutation carriers.

In mammalian cells subjected to oxidative stress, Mao et al. (2011) showed that SIRT6 (606211) is recruited to the sites of DNA double-strand breaks and stimulates double-strand break repair, through both nonhomologous end joining and homologous recombination. Mao et al. (2011) concluded that their results indicated that SIRT6 physically associates with PARP1 and mono-ADP-ribosylates PARP1 on lysine residue 521, thereby stimulating PARP1 poly-ADP-ribosylase activity and enhancing double-strand break repair under oxidative stress.

Mapping

McBride et al. (1987) concluded that a large functional PARP gene of more than 15 to 20 kb is located on chromosome 1q and that sequences on chromosomes 13 and 14 most likely represent processed pseudogenes. These localizations were achieved by probing of the DNA from panels of somatic cell hybrids.

Herzog et al. (1988) cloned a cDNA for ADPRT and localized the gene to 1q21-q22 by in situ hybridization. Herzog et al. (1989) mapped the ADPRT gene to 1q41-q42 by in situ hybridization. Using high resolution in situ hybridization techniques, Zabel et al. (1989) localized PPOL to 1q41-q42. With the conditions used, only 1 additional site of hybridization, 14q22, could be detected; this probably represented a pseudogene which had previously been identified and called ADPRTP2. By nonisotopic in situ hybridization, Baumgartner et al. (1992) confirmed localization of the functional gene to 1q42. Two other hybridization peaks, one at 13q34 and one at 14q24, suggested the location of pseudogenes.

PARP Pseudogene

A processed pseudogene or a gene with extensive identity to the ADPRT gene was studied by Bhatia et al. (1990), who mapped it to 13q33-qter. The gene was deleted in a polymorphism that was 3 times higher in frequency among blacks than Caucasians. Bhatia et al. (1990) suggested that this deletion might be a predisposing factor in several forms of malignancy.

Lyn et al. (1993) studied the 2-allele (A/B) polymorphism of the gene on 13q34. An elevated B-allele frequency was found in germline DNA in blacks with multiple myeloma (254500), prostate cancer (176807), and colon cancer (114500). They found that the A allele has a close sequence similarity (91.8%) to the PPOL cDNA coded by 1q42 and is intronless, suggesting that the gene on 13q is a processed pseudogene. They presented data indicating that the polymorphism reflects a 193-bp duplication within the processed-pseudogene sequence, with absence of this duplicated region being characteristic of the B genotype. Doll et al. (1996) confirmed the association between prostate cancer in black Americans and an allele of the pseudogene locus (which they symbolized PADPRP) on chromosome 13. Two genes in the region 13q33-q34 were viewed as potential candidates for prostate cancer: ERCC5 (133530) and RAP2A (179540).

Gene Structure

Auer et al. (1989) demonstrated that the PARP gene is 43 kb long and split into 23 exons.

Molecular Genetics

Schweiger et al. (1987) suggested that the defect in Fanconi anemia (FA; 227650) is one of impaired ADP-ribosylation. Several independent observations have suggested that ADPRT might be the site of the mutation in Fanconi anemia; however, Flick et al. (1992) could find no abnormality in cells from an FA patient of complementation group A (cell line GM6914).

Animal Model

Streptozotocin (STZ) selectively destroys insulin-producing beta islet cells of the pancreas, providing a model of type I diabetes (see 222100). PARP is a nuclear enzyme whose overactivation by DNA strand breaks depletes its substrate NAD+ and then ATP, leading to cellular death from energy depletion. Pieper et al. (1999) demonstrated DNA damage and a major activation of PARP in pancreatic islets of STZ-treated mice. These mice displayed a 5-fold increase in blood glucose and major pancreatic islet damage. In mice with homozygous targeted deletion of Parp, blood glucose and pancreatic islet structure were normal, indicating virtually total protection from STZ diabetes. Partial protection occurred in heterozygous animals. Thus, PARP activation may participate in the pathophysiology of type I diabetes, for which PARP inhibitors might afford therapeutic benefit.

Using 2 different techniques, d'Adda di Fagagna et al. (1999) showed that mice lacking PARP display telomere shortening compared with wildtype mice. Telomere shortening was seen in different genetic backgrounds and in different tissues from embryos and adult mice. In vitro telomerase activity, however, was not altered in Adprt1 −/− mouse fibroblasts. Furthermore, cytogenetic analysis of mouse embryonic fibroblasts showed that lack of PARP was associated with severe chromosomal instability, characterized by increased frequency of chromosome fusions and aneuploidy. The absence of PARP does not affect the presence of single-strand overhangs, naturally present at the end of telomeres. This study, therefore, revealed an unanticipated role of PARP in telomere length regulation and provided insight into its functions in maintaining genomic integrity.

Depletion of PARP increases the frequency of recombination, gene amplification, sister chromatid exchanges, and micronuclei formation in cells exposed to genotoxic agents, implicating PARP in the maintenance of genomic stability. By flow cytometric analysis, Simbulan-Rosenthal et al. (1999) demonstrated an unstable tetraploid population in immortalized fibroblasts derived from PARP −/− mice. There were partial chromosomal gains in other regions. Neither the chromosomal gains nor the tetraploid population were apparent in PARP −/− cells stably transfected with PARP cDNA, indicating negative selection of cells with these genetic alterations after reintroduction of PARP cDNA. These results implicated PARP in the maintenance of genomic stability.

Steroid response and stress-activated genes such as Hsp70 (see 140550) undergo puffing, a local loosening of polytene chromatin structure associated with gene induction, in Drosophila larval salivary glands. Tulin and Spradling (2003) found that puffs acquired elevated levels of ADP-ribose modified proteins and that PARP was required to produce normal-sized puffs and normal amounts of Hsp70 after heat exposure. Tulin and Spradling (2003) proposed that chromosomal PARP molecules become activated by developmental or environmental cues and strip nearby chromatin proteins off DNA to generate a puff Such local loosening may facilitate transcription and may transiently make protein complexes more accessible to modification, promoting chromatin remodeling during development.

To understand the biologic significance of PARP1 cleavage, Petrilli et al. (2004) generated a PARP1 knockin mouse model in which the caspase cleavage site of PARP1, DEVD(214), was mutated to render the protein resistant to caspases during apoptosis. The Parp1 knockin mice were highly resistant to endotoxic shock and to intestinal and renal ischemia/reperfusion, which were associated with reduced inflammatory responses in the target tissues and cells due to the compromised production of specific inflammatory mediators. Despite normal binding of nuclear factor kappa-B (see 164011) to DNA, NFKB-mediated transcription activity was impaired in the presence of caspase-resistant PARP1. Petrilli et al. (2004) concluded that the PARP1 cleavage event is physiologically relevant to the regulation of the inflammatory response in vivo.

[End of text adapted from OMIM]

### Screening for PARP-1 Binding Motif Mimics

PARP-1 Binding Motif may be used in screening for molecules which mimic the structure or activity (such as anti-PARP-1 activity) or both, of PARP-1 Binding Motif. We refer to such molecules in this document for convenience as “PARP-1 Binding Motif Mimics”. Such ARP-1 Binding Motif Mimics may be used as candidate drugs against cancer, for example breast cancer and for treating, preventing or alleviating hepatitis, for example hepatitis B, or generally for cell killing, optionally together with agents capable of killing cells or cytotoxic agents.

### Identifying PARP-1 Binding Motif Mimics

PARP-1 Binding Motif Mimics, in particular, small molecules may be used to specifically inhibit PARP-1 for use as anti-cancer, anti-hepatitis B or cell-killing agents.

We therefore disclose PARP-1 antagonists and small molecule PARP-1 Binding Motif Mimics, as well as assays for screening for these. PARP-1 Binding Motif Mimics may be screened by detecting modulation, such as down regulation or disruption, of binding of PARP-1 Binding Motif to PARP-1. Such modulation of binding may be accompanied preferably by reduction of the inhibitory effect of PARP-1 on enzymatic activity of PARP-1, such as poly (ADP-ribose) polymerase activity.

We therefore provide a compound capable of down-regulating the expression, amount or activity PARP-1. Such a compound may be used in the methods and compositions described here for treating or preventing cancer, particularly breast cancer.

PARP-1 Binding Motif may therefore be used to assess the binding of small molecule substrates and ligands in, for example, cells, cell-free preparations, chemical libraries, and natural product mixtures. These substrates and ligands may be natural substrates and ligands or may be structural or functional mimetics. See Coligan et al., Current Protocols in Immunology 1(2): Chapter 5 (1991).

In general, the assays for PARP-1 Binding Motif Mimics rely on determining the effect of candidate molecules on the binding of PARP-1 Binding Motif to PARP-1. An assay may involve assaying the binding of PARP-1 Binding Motif to PARP-1 in the presence of a candidate molecule, and optionally in the absence of the candidate molecule. Candidate molecules which disrupt, reduce, or ameliorate the binding of PARP-1 Binding Motif to PARP-1 may be selected as putative anti-cancer, anti-hepatitis or cell-killing drugs.

Another assay may involve assaying PARP-1 activity in the presence of PARP-1 Binding Motif together with a candidate molecule, and optionally in the absence of the candidate molecule. Candidate molecules which disrupt, reduce, or ameliorate the inhibitory effect of PARP-1 Binding Motif on PARP-1 activity may be selected as putative anti-cancer, anti-hepatitis or cell-killing drugs.

By “down-regulate”, “disrupt”, “reduce” or “ameliorate” we include any negative effect on the behaviour being studied; this may be total or partial. Thus, where binding is being detected, candidate antagonists are capable of reducing, ameliorating, or abolishing the binding between two entities. The down-regulation of binding (or any other activity) achieved by the candidate molecule may be at least 10%, such as at least 20%, at least 30%, at least 40%, at least 50%, at least 60%, at least 70%, at least 80%, at least 90% or more compared to binding (or which ever activity) in the absence of the candidate molecule. Thus, a candidate molecule suitable for use as an antagonist may be one which is capable of reducing by 10% more the binding or other activity.

The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. The compound may be an antibody.

Examples of potential antagonists of PARP-1 Binding Motif Mimics include antibodies, small molecules, nucleotides and their analogues, including purines and purine analogues, oligonucleotides or proteins which are closely related to PARP-1 Binding Motif, e.g., a fragment of PARP-1 Binding Motif, or small molecules which bind to the PARP-1 polypeptide so that activity of the polypeptide is prevented, etc.

### Screening Kits

The materials necessary for such screening to be conducted may be packaged into a screening kit.

Such a screening kit is useful for identifying PARP-1 Binding Motif Mimics. The screening kit may comprise: (a) a PARP-1 polypeptide; and/or (b) a PARP-1 Binding Motif. The screening kit may comprise a library. The screening kit may comprise any one or more of the components needed for screening, as described below. The screening kit may optionally comprise instructions for use.

Screening kits may also be provided which are capable of detecting PARP-1 expression at the nucleic acid level. Such kits may comprise a primer for amplification of PARP-1, or a pair of primers for amplification. The primer or primers may be chosen from any suitable sequence, for example a portion of the PARP-1 sequence. Methods of identifying primer sequences are well known in the art, and the skilled person will be able to design such primers with ease. The kits may comprise a nucleic acid probe for PARP-1 expression. The kits may also optionally comprise instructions for use.

Screening kits may also be provided which are capable of detecting enzymatic activity of PARP-1, as described elsewhere in this document.

### Rational Design

Rational design of candidate compounds likely to be able to disrupt PARP-1 Binding Motif/PARP-1 binding or inhibitory activity of PARP-1 Binding Motif on PARP-1 may be based upon structural studies of the molecular shapes of a PARP-1 Binding Motif One means for determining which sites interact with specific other proteins is a physical structure determination, e.g., X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

### Polypeptide Binding Assays

Modulators and antagonists of PARP-1 activity or expression (such as PARP-1 Binding Motif Mimics) may be identified by any means known in the art.

In their simplest form, the assays may simply comprise the steps of mixing a candidate compound with a solution containing a PARP-1 polypeptide and a PARP-1 Binding Motif to form a mixture, measuring activity of PARP-1 polypeptide in the mixture, and comparing the activity of the mixture to a standard. The standard may comprise a mixture of PARP-1 Binding Motif and PARP-1.

Furthermore, molecules may be identified by their binding to such as PARP-1, in an assay which detects binding between such as PARP-1 and the putative molecule.

One type of assay for identifying PARP-1 Binding Motif Mimics described here involves contacting the PARP-1 polypeptide, which is immobilised on a solid support and on which is bound PARP-1 Binding Motif with a non-immobilised candidate substance determining whether and/or to what extent the candidate substance disrupts or reduces the binding of PARP-1 Binding Motif and PARP-1. Alternatively, the candidate substance may be immobilised and the PARP-1/PARP-1 Binding Motif complex in solution and non-immobilised.

The binding of the substance to the PARP-1 Binding Motif Mimic to PARP-1 can be transient, reversible or permanent. The substance may bind to the PARP-1 polypeptide with a Kd value which is lower than the Kd value for binding to control polypeptides (e.g., polypeptides known to not be involved in cancer growth or progression). The Kd value of the substance may be 2 fold less than the Kd value for binding to control polypeptides, such as a Kd value 100 fold less or a Kd 1000 fold less than that for binding to the control polypeptide.

In an example assay method, the PARP-1 polypeptide may be immobilised on beads such as agarose beads. Typically this may be achieved by expressing the PARP-1 polypeptide as a GST-fusion protein in bacteria, yeast or higher eukaryotic cell lines and purifying the GST-PARP-1 fusion protein from crude cell extracts using glutathione-agarose beads (Smith and Johnson, 1988; Gene 67(10):31-40). PARP-1 Binding Motif is then bound to the immobilized GST-PARP-1 fusion protein.

The binding of the candidate substance to the immobilised PARP-1 polypeptide, including the displacement of the PARP-1 Binding Motif, may then be determined. This type of assay is known in the art as a GST pulldown assay. Again, the candidate substance may be immobilised and the PARP-1 polypeptide/PARP-1 Binding Motif complex non-immobilised.

It is also possible to perform this type of assay using different affinity purification systems for immobilising one of the components, for example Ni-NTA agarose and histidine-tagged components.

Binding of the polypeptide to the candidate substance may be determined by a variety of methods well-known in the art. For example, the non-immobilised component may be labeled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, such as from 1 to 100 nmol/ml. In the case of antibodies, the final concentration used is typically from 100 to 500 μg/ml, such as from 200 to 300 μg/ml.

Modulators and antagonists of PARP-1, including PARP-1 Binding Motif Mimics, may also be identified by detecting modulation of binding between PARP-1 and any molecule to which this polypeptide binds, such as PARP-1 Binding Motif, or modulation of any activity consequential on such binding or release.

### Cell Based Assays

A cell based assay may simply test binding of a candidate compound wherein adherence to the cells bearing the PARP-1 polypeptide is detected by means of a label directly or indirectly associated with the candidate compound or in an assay involving competition with a labeled competitor.

Further, these assays may test whether the candidate compound results in a signal generated by binding to the PARP-1 polypeptide, using detection systems appropriate to the cells bearing the polypeptides at their surfaces. Inhibitors of activation are generally assayed in the presence of a known agonist and the effect on activation by the agonist by the presence of the candidate compound is observed.

Another method of screening compounds utilises eukaryotic or prokaryotic host cells which are stably transformed with recombinant DNA molecules expressing a library of compounds. Such cells, either in viable or fixed form, can be used for standard binding-partner assays. See also Parce et al. (1989) Science 246:243-247; and Owicki et al. (1990) Proc. Nat'l Acad. Sci. USA 87; 4007-4011, which describe sensitive methods to detect cellular responses.

Competitive assays are particularly useful, where the cells expressing the library of compounds are contacted or incubated with a labelled molecule known to bind to a PARP-1 polypeptide, such as labelled PARP-1 Binding Motif, and a test sample such as a candidate compound whose binding affinity to the binding composition is being measured. The bound and free labelled binding partners for the PARP-1 polypeptide are then separated to assess the degree of binding. The amount of test sample bound is inversely proportional to the amount of labelled PARP-1 Binding Motif binding to the PARP-1 polypeptide.

Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

The assays may involve exposing a candidate molecule to a cell, such as a breast cell, and assaying expression or activity of PARP-1 by any suitable means. Molecules which down-regulate the expression of PARP-1 in such assays may be optionally chosen for further study, and used as drugs for the purposes described above. Such drugs may be usefully employed to treat or prevent breast cancer.

cDNA encoding PARP-1 protein and antibodies to the proteins may also be used to configure assays for detecting the effect of added compounds on the production of PARP-1 mRNA and protein in cells. For example, an ELISA may be constructed for measuring secreted or cell associated levels of PARP-1 polypeptide using monoclonal and polyclonal antibodies by standard methods known in the art, and this can be used to discover agents which may inhibit or enhance the production of PARP-1 protein (also called antagonist or agonist, respectively) from suitably manipulated cells or tissues. Standard methods for conducting screening assays are well understood in the art.

### Activity Assays

Assays to detect modulators or antagonists typically involve detecting modulation of any activity of PARP-1, in the presence, optionally together with detection of modulation of activity in the absence, of a candidate molecule. Such assays may optionally be conducted in the presence of PARP-1 Binding Motif.

Assays which detect specific biological activities of PARP-1, such as poly (ADP-ribose) polymerase activity, may be used. The assays typically involve contacting a candidate molecule (e.g., in the form of a library) with PARP-1 whether in the form of a polypeptide, a nucleic acid encoding the polypeptide, or a cell, organelle, extract, or other material comprising such, with a candidate modulator, in the presence of PARP-1 Binding Motif. The relevant activity of PARP-1 (such as poly (ADP-ribose) polymerase activity, as described below) may be detected, to establish whether the presence of the candidate modulator has any effect.

Assays for PARP-1 activity are known in the art. An example of such an assay follows.

### Assay for PARP-1 Activity

PARP-1 enzymatic assays were performed in triplicates using the PARP Universal Colorimetric Assay Kit (R&D Systems). 1 μg of nuclear lysates were used for determination of relative endogenous PARP-1 activity in different cell lines. 5 μg of nuclear lysates was used to test if DNA sequence-specific binding could inhibit PARP-1 activity. To obtain the test DNA duplexes, equal amounts of 100 μM DNA oligomers (1^{st} base) were mixed and incubated at 95° C. for 5 minutes, then cooled to 37° C. for 5 minutes and further cooled to 24° C. for another 5 minutes in a thermocycler. 1 μl of cooled annealed products were added per well. Lysates were dissolved in PARP buffer with PMSF and protease inhibitor cocktail (Sigma-aldrich). Assays were carried out as recommended.

The assays may be performed in the presence or absence of a candidate modulator (optionally in the presence of PARP-1 Binding Motif) and the appropriate activity detected to detect modulation of PARP-1 activity and hence identification of a candidate modulator and/or antagonist of PARP-1.

Promoter binding assays to detect candidate modulators which bind to and/or affect the transcription or expression of PARP-1 may also be used. Candidate modulators may then be chosen for further study, or isolated for use. Details of such screening procedures are well known in the art, and are for example described in, Handbook of Drug Screening, edited by Ramakrishna Seethala, Prabhavathi B. Fernandes (2001, New York, N.Y., Marcel Dekker, ISBN 0-8247-0562-9).

The screening methods described here may employ in vivo assays, although they may be configured for in vitro use. In vivo assays generally involve exposing a cell comprising PARP-1 to the candidate molecule in the presence of PARP-1 Binding Motif. In in vitro assays, PARP-1 is exposed to the candidate molecule, in the presence of PARP-1 Binding Motif and optionally in the presence of other components, such as crude or semi-purified cell extract, or purified proteins. Where in vitro assays are conducted, these may employ arrays of candidate molecules (for example, an arrayed library). In vivo assays may be employed. Therefore, the PARP-1 polypeptide may be comprised in a cell, such as heterologously. Such a cell may be a transgenic cell, which has been engineered to express PARP-1 as described above.

Where an extract is employed, it may comprise a cytoplasmic extract or a nuclear extract, methods of preparation of which are well known in the art.

It will be appreciated that any component of a cell comprising PARP-1 may be employed, such as an organelle. One embodiment utilises a cytoplasmic or nuclear preparation, e.g., comprising a cell nucleus which comprises PARP-1 as described. The nuclear preparation may comprise one or more nuclei, which may be permeabilised or semi-permeabilised, by detergent treatment, for example.

Thus, in a specific embodiment, an assay format may include the following: a multiwell microtitre plate is set up to include one or more cells expressing PARP-1 polypeptide in each well, optionally in the presence of PARP-1 Binding Motif individual candidate molecules, or pools of candidate molecules, derived for example from a library, may be added to individual wells and modulation of PARP-1 activity measured. Where pools are used, these may be subdivided in to further pools and tested in the same manner. PARP-1 activity, for example binding activity or transcriptional co-activation activity, as described elsewhere in this document may then be assayed.

Alternatively or in addition to the assay methods described above, “subtractive” procedures may also be used to identify modulators or antagonists of PARP-1. Under such “subtractive” procedures, a plurality of molecules is provided, which comprises one or more candidate molecules capable of functioning as a modulator (e.g., cell extract, nuclear extract, library of molecules, etc), and one or more components is removed, depleted or subtracted from the plurality of molecules. The “subtracted” extract, etc, is then assayed for activity, by exposure to a cell comprising PARP-1 (or a component thereof) as described.

Thus, for example, an ‘immunodepletion’ assay may be conducted to identify such modulators as follows. A cytoplasmic or nuclear extract may be prepared from a suitable cell. The extract may be depleted or fractionated to remove putative modulators, such as by use of immunodepletion with appropriate antibodies. If the extract is depleted of a modulator, it will lose the ability to affect PARP-1 function or activity or expression. A series of subtractions and/or depletions may be required to identify the modulators or antagonists.

It will also be appreciated that the above “depletion” or “subtraction” assay may be used as a preliminary step to identify putative modulatory factors for further screening. Furthermore, or alternatively, the “depletion” or “subtraction” assay may be used to confirm the modulatory activity of a molecule identified by other means (for example, a “positive” screen as described elsewhere in this document) as a putative modulator.

Candidate molecules subjected to the assay and which are found to be of interest may be isolated and further studied. Methods of isolation of molecules of interest will depend on the type of molecule employed, whether it is in the form of a library, how many candidate molecules are being tested at any one time, whether a batch procedure is being followed, etc.

The candidate molecules may be provided in the form of a library. In one embodiment, more than one candidate molecule may be screened simultaneously. A library of candidate molecules may be generated, for example, a small molecule library, a polypeptide library, a nucleic acid library, a library of compounds (such as a combinatorial library), a library of antisense molecules such as antisense DNA or antisense RNA, an antibody library etc, by means known in the art. Such libraries are suitable for high-throughput screening. Different cells comprising PARP-1 may be exposed to individual members of the library, and effect on the PARP-1 activity determined. Array technology may be employed for this purpose. The cells may be spatially separated, for example, in wells of a microtitre plate.

In an embodiment, a small molecule library is employed. By a “small molecule”, we refer to a molecule whose molecular weight may be less than about 50 kDa. In particular embodiments, a small molecule may have a molecular weight which is less than about 30 kDa, such as less than about 15 kDa or less than 10 kDa or so. Libraries of such small molecules, here referred to as “small molecule libraries” may contain polypeptides, small peptides, for example, peptides of 20 amino acids or fewer, for example, 15, 10 or 5 amino acids, simple compounds, etc.

Alternatively or in addition, a combinatorial library, as described in further detail below, may be screened for modulators or antagonists of PARP-1. Assays for PARP-1 activity are described above.

### Libraries

Libraries of candidate molecules, such as libraries of polypeptides or nucleic acids, may be employed in the screens for PARP-1 antagonists and inhibitors, such as PARP-1 Binding Motif Mimics, described here. Such libraries are exposed to PARP-1 protein, and their effect, if any, on the activity of the protein determined.

Selection protocols for isolating desired members of large libraries are known in the art, as typified by phage display techniques. Such systems, in which diverse peptide sequences are displayed on the surface of filamentous bacteriophage (Scott and Smith (1990 supra), have proven useful for creating libraries of antibody fragments (and the nucleotide sequences that encoding them) for the in vitro selection and amplification of specific antibody fragments that bind a target antigen. The nucleotide sequences encoding the V_{H} and V_{L} regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phagebodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straightforward.

Methods for the construction of bacteriophage antibody display libraries and lambda phage expression libraries are well known in the art (McCafferty et al. (1990) supra; Kang et al. (1991) Proc. Natl. Acad. Sci. U.S.A., 88: 4363; Clackson et al. (1991) Nature, 352: 624; Lowman et al. (1991) Biochemistry, 30: 10832; Burton et al. (1991) Proc. Natl. Acad. Sci U.S.A., 88: 10134; Hoogenboom et al. (1991) Nucleic Acids Res., 19: 4133; Chang et al. (1991) J. Immunol., 147: 3610; Breitling et al. (1991) Gene, 104: 147; Marks et al. (1991) supra; Barbas et al. (1992) supra; Hawkins and Winter (1992) J. Immunol., 22: 867; Marks et al., 1992, J. Biol. Chem., 267: 16007; Lerner et al. (1992) Science, 258: 1313, incorporated herein by reference). Such techniques may be modified if necessary for the expression generally of polypeptide libraries.

One particularly advantageous approach has been the use of scFv phage-libraries (Bird, R. E., et al. (1988) Science 242: 423-6, Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) supra; Marks et al. (1991) supra; Chiswell et al. (1992) Trends Biotech., 10: 80; Marks et al. (1992) supra). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys, supra), which are incorporated herein by reference.

Alternative library selection technologies include bacteriophage lambda expression systems, which may be screened directly as bacteriophage plaques or as colonies of lysogens, both as previously described (Huse et al. (1989) Science, 246: 1275; Caton and Koprowski (1990) Proc. Natl. Acad. Sci. U.S.A., 87; Mullinax et al. (1990) Proc. Natl. Acad. Sci. U.S.A., 87: 8095; Persson et al. (1991) Proc. Natl. Acad. Sci. U.S.A., 88: 2432) and are of use in the methods and compositions described here. These expression systems may be used to screen a large number of different members of a library, in the order of about 10⁶ or even more. Other screening systems rely, for example, on direct chemical synthesis of library members. One early method involves the synthesis of peptides on a set of pins or rods, such as described in WO84/03564. A similar method involving peptide synthesis on beads, which forms a peptide library in which each bead is an individual library member, is described in U.S. Pat. No. 4,631,211 and a related method is described in WO92/00091. A significant improvement of the bead-based methods involves tagging each bead with a unique identifier tag, such as an oligonucleotide, so as to facilitate identification of the amino acid sequence of each library member. These improved bead-based methods are described in WO93/06121.

Another chemical synthesis method involves the synthesis of arrays of peptides (or peptidomimetics) on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a receptor) and reactive library members occur is determined, thereby identifying the sequences of the reactive library members on the basis of spatial location. These methods are described in U.S. Pat. No. 5,143,854; WO90/15070 and WO92/10092; Fodor et al. (1991) Science, 251: 767; Dower and Fodor (1991) Ann. Rep. Med. Chem., 26: 271.

Other systems for generating libraries of polypeptides or nucleotides involve the use of cell-free enzymatic machinery for the in vitro synthesis of the library members. In one method, RNA molecules are selected by alternate rounds of selection against a target ligand and PCR amplification (Tuerk and Gold (1990) Science, 249: 505; Ellington and Szostak (1990) Nature, 346: 818). A similar technique may be used to identify DNA sequences which bind a predetermined human transcription factor (Thiesen and Bach (1990) Nucleic Acids Res., 18: 3203; Beaudry and Joyce (1992) Science, 257: 635; WO92/05258 and WO92/14843). In a similar way, in vitro translation can be used to synthesise polypeptides as a method for generating large libraries. These methods which generally comprise stabilised polysome complexes, are described further in WO88/08453, WO90/05785, WO90/07003, WO91/02076, WO91/05058, and WO92/02536. Alternative display systems which are not phage-based, such as those disclosed in WO95/22625 and WO95/11922 (Affymax) use the polysomes to display polypeptides for selection. These and all the foregoing documents also are incorporated herein by reference.

### Combinatorial Libraries

Libraries, in particular, libraries of candidate molecules, may suitably be in the form of combinatorial libraries (also known as combinatorial chemical libraries).

A “combinatorial library”, as the term is used in this document, is a collection of multiple species of chemical compounds that consist of randomly selected subunits. Combinatorial libraries may be screened for molecules which are capable of inhibiting PARP-1 such as PARP-1 Binding Motif Mimics.

Various combinatorial libraries of chemical compounds are currently available, including libraries active against proteolytic and non-proteolytic enzymes, libraries of agonists and antagonists of G-protein coupled receptors (GPCRs), libraries active against non-GPCR targets (e.g., integrins, ion channels, domain interactions, nuclear receptors, and transcription factors) and libraries of whole-cell oncology and anti-infective targets, among others. A comprehensive review of combinatorial libraries, in particular their construction and uses is provided in Dolle and Nelson (1999), Journal of Combinatorial Chemistry, Vol 1 No 4, 235-282. Reference is also made to Combinatorial peptide library protocols (edited by Shmuel Cabilly, Totowa, N.J.: Humana Press, c1998. Methods in Molecular Biology v. 87). Specific combinatorial libraries and methods for their construction are disclosed in U.S. Pat. No. 6,168,914 (Campbell, et al), as well as in Baldwin et al. (1995), “Synthesis of a Small Molecule Library Encoded with Molecular Tags,” J. Am. Chem. Soc. 117:5588-5589, and in the references mentioned in those documents.

In one embodiment, the combinatorial library which is screened is one which is designed to potentially include molecules which interact with a component of the cell to influence gene expression. For example, combinatorial libraries against chromatin structural proteins may be screened. Other libraries which are useful for this embodiment include combinatorial libraries against histone modification enzymes (e.g., histone acetylation or histone metylation enzymes), or DNA modification, for example, DNA methylation or demethylation.

Further references describing chemical combinatorial libraries, their production and use include those available from the URL http://www.netsci.org/Science/Combichem/, including The Chemical Generation of Molecular Diversity. Michael R. Pavia, Sphinx Pharmaceuticals, A Division of Eli Lilly (Published July, 1995); Combinatorial Chemistry: A Strategy for the Future—MDL Information Systems discusses the role its Project Library plays in managing diversity libraries (Published July, 1995); Solid Support Combinatorial Chemistry in Lead Discovery and SAR Optimization, Adnan M. M. Mjalli and Barry E. Toyonaga, Ontogen Corporation (Published July, 1995); Non-Peptidic Bradykinin Receptor Antagonists From a Structurally Directed Non-Peptide Library. Sarvajit Chakravarty, Babu J. Mavunkel, Robin Andy, Donald J. Kyle*, Scios Nova Inc. (Published July, 1995); Combinatorial Chemistry Library Design using Pharmacophore Diversity Keith Davies and Clive Briant, Chemical Design Ltd. (Published July, 1995); A Database System for Combinatorial Synthesis Experiments—Craig James and David Weininger, Daylight Chemical Information Systems, Inc. (Published July, 1995); An Information Management Architecture for Combinatorial Chemistry, Keith Davies and Catherine White, Chemical Design Ltd. (Published July, 1995); Novel Software Tools for Addressing Chemical Diversity, R. S. Pearlman, Laboratory for Molecular Graphics and Theoretical Modeling, College of Pharmacy, University of Texas (Published June/July, 1996); Opportunities for Computational Chemists Afforded by the New Strategies in Drug Discovery: An Opinion, Yvonne Connolly Martin, Computer Assisted Molecular Design Project, Abbott Laboratories (Published June/July, 1996); Combinatorial Chemistry and Molecular Diversity Course at the University of Louisville: A Description, Arno F. Spatola, Department of Chemistry, University of Louisville (Published June/July, 1996); Chemically Generated Screening Libraries: Present and Future. Michael R. Pavia, Sphinx Pharmaceuticals, A Division of Eli Lilly (Published June/July, 1996); Chemical Strategies For Introducing Carbohydrate Molecular Diversity Into The Drug Discovery Process. Michael J. Sofia, Transcell Technologies Inc. (Published June/July, 1996); Data Management for Combinatorial Chemistry. Maryjo Zaborowski, Chiron Corporation and Sheila H. DeWitt, Parke-Davis Pharmaceutical Research, Division of Warner-Lambert Company (Published November, 1995); and The Impact of High Throughput Organic Synthesis on R&D in Bio-Based Industries, John P. Devlin (Published March, 1996).

Techniques in combinatorial chemistry are gaining wide acceptance among modern methods for the generation of new pharmaceutical leads (Gallop, M. A. et al., 1994, J. Med. Chem. 37:1233-1251; Gordon, E. M. et al., 1994, J. Med. Chem. 37:1385-1401.). One combinatorial approach in use is based on a strategy involving the synthesis of libraries containing a different structure on each particle of the solid phase support, interaction of the library with a soluble receptor, identification of the ‘bead’ which interacts with the macromolecular target, and determination of the structure carried by the identified ‘bead’ (Lam, K. S. et al., 1991, Nature 354:82-84). An alternative to this approach is the sequential release of defined aliquots of the compounds from the solid support, with subsequent determination of activity in solution, identification of the particle from which the active compound was released, and elucidation of its structure by direct sequencing (Salmon, S. E. et al., 1993, Proc. Natl. Acad. Sci. USA 90:11708-11712), or by reading its code (Kerr, J. M. et al., 1993, J. Am. Chem. Soc. 115:2529-2531; Nikolaiev, V. et al., 1993, Pept. Res. 6:161-170; Ohlmeyer, M. H. J. et al., 1993, Proc. Natl. Acad. Sci. USA 90:10922-10926).

Soluble random combinatorial libraries may be synthesized using a simple principle for the generation of equimolar mixtures of peptides which was first described by Furka (Furka, A. et al., 1988, Xth International Symposium on Medicinal Chemistry, Budapest 1988; Furka, A. et al., 1988, 14th International Congress of Biochemistry, Prague 1988; Furka, A. et al., 1991, Int. J. Peptide Protein Res. 37:487-493). The construction of soluble libraries for iterative screening has also been described (Houghten, R. A. et al. 1991, Nature 354:84-86). K. S. Lam disclosed the novel and unexpectedly powerful technique of using insoluble random combinatorial libraries. Lam synthesized random combinatorial libraries on solid phase supports, so that each support had a test compound of uniform molecular structure, and screened the libraries without prior removal of the test compounds from the support by solid phase binding protocols (Lam, K. S. et al., 1991, Nature 354:82-84).

Thus, a library of candidate molecules may be a synthetic combinatorial library (e.g., a combinatorial chemical library), a cellular extract, a bodily fluid (e.g., urine, blood, tears, sweat, or saliva), or other mixture of synthetic or natural products (e.g., a library of small molecules or a fermentation mixture).

A library of molecules may include, for example, amino acids, oligopeptides, polypeptides, proteins, or fragments of peptides or proteins; nucleic acids (e.g., antisense; DNA; RNA; or peptide nucleic acids, PNA); aptamers; or carbohydrates or polysaccharides. Each member of the library can be singular or can be a part of a mixture (e.g., a compressed library). The library may contain purified compounds or can be “dirty” (i.e., containing a significant quantity of impurities).

Commercially available libraries (e.g., from Affymetrix, ArQule, Neose Technologies, Sarco, Ciddco, Oxford Asymmetry, Maybridge, Aldrich, Panlabs, Pharmacopoeia, Sigma, or Tripose) may also be used with the methods described here.

In addition to libraries as described above, special libraries called diversity files can be used to assess the specificity, reliability, or reproducibility of the new methods. Diversity files contain a large number of compounds (e.g., 1000 or more small molecules) representative of many classes of compounds that could potentially result in nonspecific detection in an assay. Diversity files are commercially available or can also be assembled from individual compounds commercially available from the vendors listed above.

Agents which Facilitate Cell Death

We describe a combination of a PARD-1 Binding Motif together with an agent which facilitates cell death. The agent which facilitates cell death may comprise a cytotoxic agent.

Such a combination of a PARP-1 Binding Motif together with an agent which facilitates cell death may be used to kill a cell such as a cancer cell.

An agent which is capable of facilitating cell death is one which, when exposed to a target cell, tissue or tissue mass, is necessary or sufficient to cause cell death. Such an agent may be one which enhances the cell killing ability of treatment with PARP-1 Binding Motif Contact with such an agent therefore preferably enhances the cell killing, disruption or ablation effected by PARP-1 Binding Motif. In a preferred embodiment, cell-death facilitating agents are used to “mop up” and destroy any cells which have, whether inadvertently or on purpose, been unaffected by the treatment, with PARP-1 Binding Motif.

Such facilitating agents may have the ability to promote cell death on their own. Indeed, any agent used for treatment of tumours or cancers as known in the art is a suitable candidate for use as a cell-death facilitating agent. However, the term “agent which facilitates cell death” and “cell death facilitating agent” should be taken to include those agents which work to promote cell death when used in combination with PARP-1 Binding Motif, as set out above. Preferably, the administration of such cell death facilitating agents enhances cell killing by more than 10%, more than 20%, more than 30%, more than 40%, more than 50%, more than more than 60%, more than 70%, more than 80%, more than 90%, more than 100% compared to the cell killing efficiency of the treatments (e.g., PARP-1 Binding Motif) alone.

The cell-death facilitating agent may work in any number of ways. For example, the agent may be directly toxic to the cell. Agents in this category include cytotoxics, which are used in tumour therapy. The agent may further be one which causes an immune reaction in the host, to the effect that the target cell, tissue, etc is eliminated or killed by the patient's normal immune processes (including both cell-mediated and humoural immune responses). Examples of such agents include cytotoxic T cells and dendritic cells. Furthermore, the agent may comprise an agent which is capable of recruiting immune responses, such as a cytokine. For example, cytokines such as IL-2, GMCSF etc may be used to promote the host's immune response.

The cell-death facilitating agent may be applied directly to the cell, or in its vicinity. It will be appreciated that, where the cell-death facilitating agent is proteinaceous in nature (e.g., a peptide, a polypeptide, a protein, or a fragment of any of these), the cell-death facilitating agent may be administered in the form of a nucleic acid encoding the peptide, polypeptide, etc. Such a nucleic acid may preferably be in the form of an expression vector, and methods for making such vectors and constructs, and the induction of expression from these, are known in the art. Furthermore, we envisage the use of cell-death facilitating agents in the form of cells producing such agents, for example, a cell capable of expressing a cell-death facilitating agent by virtue of comprising a nucleic acid sequence encoding that agent. The cell may be transfected or transformed with a nucleic acid, for example, an expression vector, encoding the cell death facilitating agent, for example, cytotoxic agent, a cytostatic agent, a cytokine, GM-CSF, IL-2 or an immunogen.

The cell-death facilitating agent need not necessarily be molecular in nature. Thus, the use of treatments which cause cell killing by other means, as known in the art, is also encompassed. Examples include the use of radiation, whether applied externally or internally, to kill cells such as tumour cells. Methods of using radiotherapy as primary or auxiliary therapy for tumours is known in the art.

The target cell, tissue or tissue mass may be exposed to the cell-death facilitating agent either before, during or after PARP-1 Binding Motif.

The cell-death facilitating agent may be exposed to the target cell, tissue or tissue mass by any suitable manner. For example, the agent may be topically applied to the skin; this is advantageous if for example the target cell is epidermal (for example, a skin tumour). Furthermore, the agent may be systemically administered to the patient or to the system of which the target cell, etc forms a part. The agent may be administered orally (taken by mouth), nasally, delivered using liposome technology, etc. The agent may be directly injected into the tumour mass, or at or near the tumour site. The agent may be delivered in the form of a cell expressing the agent, for example a cell expressing a cytotoxic agent. Use of cells expressing such cytotoxic agents, for example, IL-2, is known in the art, and described in for example, Mir et al., J. Immunotherapy, 17, 30-38 and Orlowski, et al., 1998, Anticancer Drugs 9, 551-556.

The agent may be delivered by being loaded into a suitable carrier. The agent may be delivered into an intracellular compartment by the use of Membrane Translocation Sequences (MTS). The agent may also suitably be administered in the form of a pharmaceutical composition, as described in further detail below.

Agents useful in the methods and compositions described here are set out below. Preferred agents which are capable of facilitating cell death include cytokines and cytotoxics, as well as nucleic acids encoding these. These are discussed in further detail elsewhere in this document.

As used herein, the term “agent” includes but is not limited to an atom or molecule, wherein a molecule may be inorganic or organic, a biological effector molecule and/or a nucleic acid encoding an agent such as a biological effector molecule, a protein, a polypeptide, a peptide, a nucleic acid, a peptide nucleic acid (PNA), a virus-like particle, a nucleotide, a deoxyribonucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid, a fatty acid and a carbohydrate. An agent may be in solution or in suspension (e.g., in crystalline, colloidal or other particulate form). The agent may be in the form of a monomer, dimer, oligomer, etc, or otherwise in a complex. The agent may be coated with one or more molecules, preferably macromolecules, most preferably polymers such as PEG (polyethylene glycol). Use of a PEGylated agent increases the circulating lifetime of the agent once released.

The cell death facilitating agent may be radioactive, i.e., a radionuclide which is used in radiotherapy. The radionuclide may be a radio-isotope as known in the art, for example cobalt-60, iodine-131, etc, or a molecule such as a nucleic acid, polypeptide, or other molecule as explained below conjugated with such a radio-isotope. As noted above, external radiation sources utilising such radionuclides, in the form of external and/or internal radiotherapy may also be used to facilitate cell death in the treated target cell or tissue.

It will be appreciated that it is not necessary for a single agent to be used, and that it is possible to utilise two or more cell death facilitating agents, sequentially or simultaneously, to achieve cell death. Accordingly, the term “agent” also includes mixtures, fusions, combinations and conjugates, of atoms, molecules etc as disclosed herein. For example, an agent may include but is not limited to: a nucleic acid combined with a polypeptide; two or more polypeptides conjugated to each other; a protein conjugated to a biologically active molecule (which may be a small molecule such as a prodrug); or a combination of a biologically active molecule with an imaging agent.

As used herein, the term “biological effector molecule” or “biologically active molecule” refers to an agent that has activity in a biological system, including, but not limited to, a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof may be natural, synthetic or humanised, a peptide hormone, a receptor, and a signalling molecule. Included within the term “immunoglobulin” are intact immunoglobulins as well as antibody fragments such as Fv, a single chain Fv (scFv), a Fab or a F(ab′)₂.

Preferred immunoglobulins, antibodies, Fv fragments, etc are those which are capable of binding to antigens in an intracellular environment, known as “intrabodies” or “intracellular antibodies”. An “intracellular antibody” or an “intrabody” is an antibody which is capable of binding to its target or cognate antigen within the environment of a cell, or in an environment which mimics an environment within the cell.

Selection methods for directly identifying such “intrabodies” have been proposed, such as an in vivo two-hybrid system for selecting antibodies with binding capability inside mammalian cells. Such methods are described in International Patent Application number PCT/GB00/00876, hereby incorporated by reference. Techniques for producing intracellular antibodies, such as anti-β-galactosidase scFvs, have also been described in Martineau, et al., 1998, J Mol Biol 280, 117-127 and Visintin, et al., 1999, Proc. Natl. Acad. Sci. USA 96, 11723-11728.

An agent may include a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. If the biological effector molecule is a polypeptide, it may be applied directly to the target area; alternatively, a nucleic acid molecule bearing a sequence encoding the polypeptide, which sequence is operatively linked to transcriptional and translational regulatory elements active in a cell at the target site, may be used. Small molecules, including inorganic and organic chemicals, are also of use. In a particularly preferred embodiment, the biologically active molecule is a pharmaceutically active agent, for example, an isotope.

A preferred embodiment comprises use of a ribozyme or an oligonucleotide such as an antisense oligonucleotide and exposing this to a target cell or tissue to facilitate cell death.

Particularly useful classes of biological effector molecules include, but are not limited to, antibiotics, anti-inflammatory drugs, angiogenic or vasoactive agents, growth factors and cytotoxic agents (e.g., tumour suppressers). Cytotoxic agents of use include, but are not limited to, diptheria toxin, Pseudomonas exotoxin, cholera toxin, pertussis toxin, and the prodrugs peptidyl-p-phenylenediamine-mustard, benzoic acid mustard glutamates, ganciclovir, 6-methoxypurine arabinonucleoside (araM), 5-fluorocytosine, glucose, hypoxanthine, methotrexate-alanine, N-[4-(a-D-galactopyranosyl) benyloxycarbonyl]-daunorubicin, amygdalin, azobenzene mustards, glutamyl p-phenylenediamine mustard, phenolmustard-glucuronide, epirubicin-glucuronide, vinca-cephalosporin, phenylenediamine mustard-cephalosporin, nitrogen-mustard-cephalosporin, phenolmustard phosphate, doxorubicin phosphate, mitomycin phosphate, etoposide phosphate, palytoxin-4-hydroxyphenyl-acetamide, doxorubicin-phenoxyacetamide, melphalan-phenoxyacetamide, cyclophosphamide, ifosfamide or analogues thereof. If a prodrug is applied to the target cell, tissue or tissue mass in inactive form, a second biological effector molecule may be applied. Such a second biological effector molecule is usefully an activating polypeptide which converts the inactive prodrug to active drug form, and which activating polypeptide is selected from the group that includes, but is not limited to, viral thymidine kinase (encoded by Genbank Accession No. J02224), carboxypeptidase A (encoded by Genbank Accession No. M27717), α-galactosidase (encoded by Genbank Accession No. M13571), β-glucuronidase (encoded by Genbank Accession No. M15182), alkaline phosphatase (encoded by Genbank Accession No. J03252 J03512), or cytochrome P-450 (encoded by Genbank Accession No. D00003 N00003), plasmin, carboxypeptidase G2, cytosine deaminase, glucose oxidase, xanthine oxidase, β-glucosidase, azoreductase, t-gutamyl transferase, β-lactamase, or penicillin amidase. Either the polypeptide or the gene encoding it may be administered; if the latter, both the prodrug and the activating polypeptide may be encoded by genes on the same recombinant nucleic acid construct.

Preferably the biological effector molecule is selected from the group consisting of a protein, a polypeptide, a peptide, a nucleic acid, a virus-like particle, a nucleotide, a ribonucleotide, a synthetic analogue of a nucleotide, a synthetic analogue of a ribonucleotide, a modified nucleotide, a modified ribonucleotide, an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically).

The biological effector molecule is preferably an immunomodulatory agent or other biological response modifier. Also included are polynucleotides which encode metabolic enzymes and proteins, including antiangiogenesis compounds, e.g., Factor VIII or Factor IX.

### Cytotoxics

The cell-death facilitating agents described above may be used, whether alone or in combination with each other, together with PARP-1 Binding Motif treatment. Preferred cell-death facilitating agents which are delivered include cytotoxics and cytokines.

“Cytotoxicity” refers to the cell killing property of a chemical compound (such as a food, cosmetic, or pharmaceutical) or a mediator cell (cytotoxic T cell). In contrast to necrosis and apoptosis, the term cytotoxicity need not necessarily indicate a specific cellular death mechanism. For example, cell mediated cytotoxicity (that is, cell death mediated by either cytotoxic T lymphocytes [CTL] or natural killer [NK] cells) combines some aspects of both necrosis and apoptosis. The terms “cytotoxic” and “cytoxic drug” are used interchangeably, and refer to any of a group of drugs that are toxic to cells and cause cell death or prevent any cell process such as cell growth, proliferation, or replication. The latter are also referred to as “cytostats” or “cytostatic drugs”.

Preferably, the cytotoxic comprises chemotherapeutic agents having an antitumor effect. Cytotoxics are used mainly to treat cancer, although some have other uses (such as for treatment of other disorders, such as psoriasis and rheumatoid arthritis). Cancer treatment with cytotoxics is known as chemotherapy and has a variety of purposes. The cytotoxics may be used to shrink a tumour before surgery (neoadjuvant chemotherapy); they may be used after the primary tumour has been treated with surgery or radiotherapy to prevent the spread and growth of secondary tumours (adjuvant chemotherapy), or they may be the main treatment for the disease. Chemotherapy may be given to cure the disease or, if cure is not possible, to control its symptoms (palliative chemotherapy).

Cytotoxics suitable for use for preferred embodiments include alkylating drugs, antimetabolites, vinca alkaloids, cytotoxic antibiotics, platinum compounds (e.g. carboplatin), taxanes, topoisomerase inhibitors, procarbazine, crisantaspase, hydroxyurea, Rituximab (a monoclonal antibody) and aldesleukin (an interleukin) Other preferred examples of cytotoxics include bleomycin, neocarcinostatin, suramin, doxorubicin, carboplatin, taxol, mitomycin C, cisplatin, Azathioprine, (Imuran), Cyclophosphamide, (Cytoxan), Methotrexate (Rheumatrex), as well as other cytotoxic drugs related to cyclophosphamide (Cytoxan) including chlorambucil (Leukeran) and nitrogen mustard (Mustargen).

Sex hormones have been used to treat cancer, and may also be used. Tumours of the prostate gland are often stimulated by male sex hormones (the androgens) and so these cancers may be treated with oestrogens (to oppose the androgens) or with anti-androgens. Analogues of gonadorelin, such as buserelin, goserelin, leuprorelin, and triptorelin, may also be used. Some breast cancers are stimulated by oestrogens; such cancers respond to the oestrogen antagonists tamoxifen and toremifene or to aromatase inhibitors. Any of the above cytotoxics may be employed in the preferred methods described here.

Also included within the term “cytotoxic” are cells such as Cytotoxic T lymphocytes (CTL) and Natural Killer (NK) cells. Furthermore, it has been found that compounds which inhibit the effects of VEGF, such as PTK787/ZK 222584, have the potential to provide effective and well-tolerated therapies for the treatment of solid tumours (Wood J M, 2000, Medicina (B Aires) 60 Suppl 2:41-7). Accordingly, the use of such compounds as cell-death facilitating agents is also envisaged.

The cytotoxic may be taken by mouth or given by injection or infusion. In general, cytotoxics may be administered in any suitable manner. Preferred routes of administration include administration systemically, orally and nasally. A highly preferred route is local administration to the target tissue. Any suitable formulation, as disclosed in further detail below, may be employed. A combination of two, three, or more cytotoxics, optionally together with one, two, three or more cytokines (as disclosed in further detail elsewhere) may be given. The effects of cytotoxics may need to be carefully monitored and blood tests carried out regularly.

### PARP-1 Polypeptides

PARP-1 polypeptides may be used for a variety of means. The activity of PARP-1 may be reduced or inhibited, such as by use of PARP-1 Binding Motif sequences, for inhibition of hepatitis B virus replication, or for killing a cancer cell, such as a breast cancer cell.

A “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-inking, cyclization, disulfide bond formation, demethylation, formation of covalent cross-inks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al, “Protein Synthesis: Posttranslational Modifications and Aging”, Ann NY Acad Sci (1992) 663:48-62.

The term “polypeptide” includes the various synthetic peptide variations known in the art, such as a retroinverso D peptides. The peptide may be an antigenic determinant and/or a T-cell epitope. The peptide may be immunogenic in vivo. The peptide may be capable of inducing neutralising antibodies in vivo.

As applied to PARP-1, the resultant amino acid sequence may have one or more activities, such as biological activities in common with a PARP-1 polypeptide, for example a human PARP-1 polypeptide. In particular, the term “homologue” covers identity with respect to structure and/or function providing the resultant amino acid sequence has PARP-lactivity. With respect to sequence identity (i.e. similarity), there may be at least 70%, such as at least 75%, such as at least 85%, such as at least 90% sequence identity. There may be at least 95%, such as at least 98%, sequence identity. These terms also encompass polypeptides derived from amino acids which are allelic variations of the PARP-1 nucleic acid sequence.

Where reference is made to the “activity” or “biological activity” of a polypeptide such as PARP-1, these terms are intended to refer to the metabolic or physiological function of PARP-1, including similar activities or improved activities or these activities with decreased undesirable side effects. Also included are antigenic and immunogenic activities of PARP-1. Examples of such activities, and methods of assaying and quantifying these activities, are known in the art, and are described in detail elsewhere in this document.

### Treatment of Cancer

As described above, PARP-1 Binding Motif may be used to kill a cancer cell or to treat cancer. The cancer may comprise breast cancer, such as BRCA-1 or BRCA-2 (or both) deficient breast cancer.

The methods and compositions described here suitably enable an improvement in a measurable criterion in an individual to whom the treatment is applied, compared to one who has not received the treatment.

For this purpose, a number of criteria may be designated, which reflect the progress of cancer or the well-being of the patient. Useful criteria may include tumour size, tumour dimension, largest dimension of tumour, tumour number, presence of tumour markers (such as alpha-feto protein), degree or number of metastates, etc.

Thus, as an example, a treated individual may show a decrease in tumour size or number as measured by an appropriate assay or test. A treated individual may for example show a 1%, 2%, 3%, 4%, 5%, 6%, 7%, 8%, 9%, 10%, 15%, 20%, 25%, 30%, 40%, 50%, 60%, 70%, 80%, 90%, 100% or more decrease in tumour size of a particular tumour, or decrease in tumour number, or both, compared to an individual who has not been treated.

In some embodiments, the effect of the treatment is suitably quantified using standard tests, such as the international criteria proposed by the Response Evaluation Criteria in Solid Tumours (RECIST) Committee, as described in detail in Therasse, P., S. G. Arbuck, et al. (2000). “New guidelines to evaluate the response to treatment in solid tumors. European Organization for Research and Treatment of Cancer, National Cancer Institute of the United States, National Cancer Institute of Canada.” J Natl Cancer Inst 92(3): 205-16.

In other embodiments, the effect of the treatment may be quantified by following the administration and testing protocols described in the Clinical Trial (Examples E1 to E8). Thus, assessment of the effect of the treatment may be carried out using one or more of the protocols, preferably all, as set out in Example E8: Measurement of Effect. Where this is the case, the treatment may result in a Partial Response (PR) or a Complete Response (CR).

Although the controls described above have been described as individuals who have not received treatment, in some cases, a more suitable control may be the patient himself, prior to receiving treatment.

For the purposes of this document, the term “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

For example, the cancer may comprise breast cancer.

### Pharmaceutical Compositions

The PARP-1 Binding Motif sequences may be effective in treating hepatitis B infection and cancer related diseases.

We disclose a method of treating cancer related disease with an effective amount of a PARP-1 Binding Motif described here. The PARP-1 Binding Motif sequences may be provided as isolated and substantially purified proteins and protein fragments in pharmaceutically acceptable compositions using formulation methods known to those of ordinary skill in the art.

The PARP-1 Binding Motif sequences may be administered in the form of a pharmaceutical composition. Such a pharmaceutical composition may include a therapeutically effective amount of PARP-1 Binding Motif sequence, together with a suitable excipient, diluent or carrier.

The PARP-1 Binding Motif sequence may in particular be introduced into the circulation of a patient, for example by being injected into a patient via, e.g., a vein.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

These compositions can be administered by standard routes. These include but are not limited to: oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) transdermal, intraperitoneal, intracranial, intracerebroventricular, intracerebral, intravaginal, intrauterine, or parenteral (e.g., intravenous, intraspinal, subcutaneous or intramuscular) routes.

The PARP-1 Binding Motif sequence formulations may conveniently be presented in unit dosage form and may be prepared by conventional pharmaceutical techniques. Such techniques include the step of bringing into association the active ingredient and the pharmaceutical carriers) or excipient(s). In general, the formulations are prepared by uniformly and intimately bringing into association the active ingredient with liquid carriers or finely divided solid carriers or both, and then, if necessary, shaping the product.

In addition, the PARP-1 Binding Motif sequences may be incorporated into biodegradable polymers allowing for sustained release of the compound, the polymers being implanted in the vicinity of where drug delivery is desired, for example, at the site of a tumor or implanted so that the PARP-1 Binding Motif sequence is slowly released systemically. The biodegradable polymers and their use are described, for example, in detail in Brem et of (1. Neurosurg 1991 74:441-446). Osmotic minipumps may also be used to provide controlled delivery of high concentrations of PARP-1 Binding Motif sequences through cannulae to the site of interest, such as directly into a metastatic growth or into the vascular supply to that tumor.

The PARP-1 Binding Motif sequence may be linked to cytotoxic agents which are infused in a manner designed to maximize delivery to the desired location. For example, ricin-linked high affinity PARP-1 Binding Motif sequences are delivered through a cannula into vessels supplying the target site or directly into the target. Such agents are also delivered in a controlled manner through osmotic pumps coupled to infusion cannulae.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations described here may include other agents conventional in the art having regard to the type of formulation in question.

The PARP-1 Binding Motif sequence conjugates may be administered in any suitable way. usually parenterally, for example intravenously or intraperitoneally, in standard sterile, non-pyrogenic formulations of diluents and carriers, for example isotonic saline (when administered intravenously). Once the PARP-1 Binding Motif sequence conjugate has bound to the target cells and been cleared from the bloodstream (if necessary), which typically takes a day or so, the pro-drug is administered, usually as a single infused dose, or the tumour is imaged. If needed, because the PARP-1 Binding Motif sequence conjugate may be immunogenic, cyclosporin or some other immunosuppressant can be administered to provide a longer period for treatment but usually this will not be necessary.

The dosage of the PARP-1 Binding Motif sequence described here will depend on the disease state or condition being treated and other clinical factors such as weight and condition of the human or animal and the route of administration of the compound.

Depending upon the half-life of the PARP-1 Binding Motif sequence in the particular animal or human, the PARP-1 Binding Motif sequence can be administered between several times per day to once a week. It is to be understood that the methods and compositions described here have application for both human and veterinary use. The methods described here contemplate single as well as multiple administrations, given either simultaneously or over an extended period of time.

The timing between administrations of the PARP-1 Binding Motif sequence conjugate and pro-drug may be optimised in a routine way since tumour/normal tissue ratios of conjugate (at least following intravenous delivery) are highest after about 4-6 days, whereas at this time the absolute amount of conjugate bound to the tumour, in terms of percent of injected dose per gram, is lower than at earlier times.

Therefore, the optimum interval between administration of the PARD-1 Binding Motif sequence conjugate and the pro-drug will be a compromise between peak tumour concentration of enzyme and the best distribution ratio between tumour and normal tissues. The dosage of the PARP-1 Binding Motif sequence conjugate will be chosen by the physician according to the usual criteria. At least in the case of methods employing a targeted enzyme such as β-glucosidase and intravenous amygdalin as the toxic pro-drug, 1 to 50 daily doses of 0.1 to 10.0 grams per square metre of body surface area, preferably 1.0-5.0 g/m² are likely to be appropriate. For oral therapy, three doses per day of 0.05 to 10.0 g, preferably 1.0-5.0 g, for one to fifty days may be appropriate. The dosage of PARP-1 Binding Motif sequence conjugate will similarly be chosen according to normal criteria, particularly with reference to the type, stage and location of the tumour and the weight of the patient. The duration of treatment will depend in part upon the rapidity and extent of any immune reaction to the PARP-1 Binding Motif sequence conjugate.

### Diseases

PARP-1 Binding Motif sequences described here, for example in the form of pharmaceutical compositions, may be used in the treatment of cancer.

For the purposes of this document, the term “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

PARP-1 Binding Motif sequences described here, for example in the form of pharmaceutical compositions, can also be used in the treatment of cancer related disorders.

Such disorders include but not limited to: solid tumours; blood born tumours such as leukemias; tumor metastasis; benign tumours, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; wound granulation; coronary collaterals; cerebral collateralsl arteriovenous malformations; ischemic limb angiogenesis; neovascular glaucoma; retrolental fibroplasia; diabetic neovascularisation; helicobacter related diseases, fractures, vasculogenesis, hematopoiesis, ovulation, menstruation and placentation.

### EXAMPLES

### Examples Part 1

### Reduction of PARP-1 Expression Inhibits HBV Replication

### Example 1

### Materials and Methods—Cell Culture

HepG2 was maintained at 37° C., 5% (v/v) CO₂ in humidified atmosphere with DMEM (Dulbecco's modified Eagle's medium) supplemented with 10% (v/v) fetal calf serum in the absence of antibiotics.

### Example 2

### Materials and Methods—Plasmid Constructs and siRNA

The HBV core promoter deletions were cloned using a multi-step strategy. First, the wild-type core promoter from HBV genotype A was inserted via KpnI and HindIII sites on the PGL3 Basic vector. Using this as the template, PGLF (5′ TCCCCAGTGCAAGTGCAGG) was used with reverse primer BX, where “X” denotes the deletion number and contains the nucleotide sequence flanking the 3′ end of the deletion. This amplifies the 5′ half of mutant core promoter. Primer CX containing nucleotide sequences flanking the 5′ end of the deletion site was then used with reverse primer PGLR (5′ TTTGGCGTCTTCCATGGTGGC) to synthesize the 3′ end of the mutant promoter. The 2 halves were annealed via their complementary sequences and amplified using primers PGLF and PGLR. PCR was performed using the Expand High Fidelity PCR System (Roche), and ran on 1% agarose gels in TBE buffer. DNA of appropriate sizes were extracted using the Qiaquick Gel Extraction Kit (Qiagen), double-digested with enzymes from New England BioLabs, then annealed using the Quick Ligation Kit.

Construction of Wild-Type PARP-1 Plasmid

A PARP-1 over-expression vector was synthesized as follows. Total RNA was extracted from Huh-7 using the NucleoSpin® RNA II kit (Machery Nagel) as directed. RNA concentration was determined spectrophotometrically. RNA with A_{260:280} of between 1.98 and 2.02 was used for reverse transcription using the Accuscript® High Fidelity 1^{st} Strand cDNA Synthesis Kit (Stratagene) with 100 ng total RNA and oligo-dT primers as instructed. The coding sequence of PARP-1 was amplified using the Expand Long Template PCR System (Roche) with the following primers:

PARP-1-F: 5′TTAGCTAGCATGGCGGAGTCTTCGG
PARP-1-R: 5′AAGCTCGAGCTACCTCTCCCAATTACC

The coding sequence of PARP-1 was analyzed with the following sequencing primers:

PARP-1-1: 5′TTGCTACAGAGGATAAAGAAGCCC
PARP-1-2: 5′ATCAGCACC AAAAAGGAGGTGG
PARP-1-3: 5′AAAGCCATGGTGGAGTATGAGATC
PARP-1-4: 5′TGGAAACATGTATGAACTGAAGCACG

No mutations were found. The wild-type PARP-1 PCR product from Huh-7 was then inserted into pcDNA3.1+ via the NheI and XhoI restriction sites.

To investigate the effect of single base mutations in URR17 and the effect of single amino acid substitutions in PARP-1 domains, mutations were carried out, each base was mutated with the QuickChange® II Site-Directed Mutagenesis Kit (Stratagene) as per instructions.

Full-length replicative HBV genotype A (approximately 1.1X HBV genome starting from nucleotide 1535, ending at nucleotide 1930) driven by its own core promoter cloned from another construct and inserted into pcDNA3.1+ via the MfeI and MluI restriction sites, upstream of the CMV promoter. Primers used are as follows:

HBV-MfeIF: 5′TGCCAATTGTTTACGCGGTCTCCCC
HBV-MluIR: 5′TGCACGCGTGCTCCAAATTCTTTATAAGG

The coding sequence for red fluorescent protein (RFP) was cut from the pTurboFP635N (Evrogen) and inserted into the pcDNA3.1+ vector containing the HBV construct into the MCS via KpnI and NotI sites.

The effect of PARP-1 sequence specific binding on PARP-1 dependent DNA repair was tested with the PARP-1 motif construct. Forward (Test-F) and reverse (Test-R) oligomers (1^{st} base) were synthesized and annealed in a thermocycler with the following parameters: 95° C. 5 minutes, 60° C. 5 minutes, 25° C. 5 minutes. The annealed product was then inserted into pcDNA3.1+ via MfeI and MluI restriction sites. The sequence-dependent requirement of the PARP-1 motif was tested with the mutant motif construct generated by the same approach using primers Mutate-F and Mutate-R. Sequences for the inserted oligomers are as follows:

Test-F: 5′AATTGTACATCAAAGTACATCAAAGTACATCAAAGA
Test-R: 5′CGCGTCTTTGATGTACTTTGATGTACTTTGATGTAC
Mutate-F: 5′AATTGTACAGGCCAGTACAGGCCAGTACAGGCCAGA
Mutate-R: 5′CGCGTCTGGCCTGTACTGGCCTGTACTGGCCTGTAC

PARP-1 specific knock-down was achieved with Silencer® Select Validated siRNA #s1098 (Ambion). The Silencer® Select Negative Control #2 siRNA (Ambion) was used as a non-specific siRNA control.

### Example 3

### Materials and Methods—Luciferase Assays

Assays were performed with the Dual-Luciferase® Reporter Assay System (Promega) with modifications. HepG2 cells were seeded 18 hours prior to transfection in 24-well plates in triplicates at a density of 1.5×10⁵ cells per well. Transfection was performed with 2.2 μl Lipofectamine 2000 (Invitrogen), 1 μg of PGL3 Basic construct plasmid and 15 ng of Renilla-luciferase construct per well. Additionally, 250 ng of over-expression plasmids for PARP-1 mutants were used when indicated. Transfected cells were lysed with 60 μl lysis buffer at 37° C. on a shaking incubator for 20 minutes after washing thrice with ice-cold PBS 30 hours post-transfection and 50 μl of the lysate was transferred on ice into black 96-well plates with clear bottoms (Corning), and luminescence measured with the GloMax® Multi-Microplate Multimode Reader (Promega).

### Example 4

### Materials and Methods—Multiple Sequence Alignments

For the alignment of known PARP-1 sequence-specific sites, the reported sequences of probes that have been validated were globally aligned with the Clustal X version 2.0.5, with reiteration until all reported polymorphisms and loss of PARP-1 dependent activity by mutations may be accounted for. The 8-nucleotide sequence corresponding to that of the HBV PARP-1 dependent sequence was then used for frequency plot using WebLogo which may be assessed using the following URL: http://weblogo.berkeley.edu/logo.cgi

### Example 5

### Materials and Methods—Protein Extraction, Western Blots, Immunofluorescence Staining and Antibodies

Nuclear lysates were obtained using the NE-PER kit (Thermo Scientific) as recommended. Lysis was performed on ice in the presence of PMSF and protease inhibitor cocktail (Sigma-aldrich). Protein concentration was determined using 1 μl lysate with the Bradford assay. Unless used immediately, lysates were stored at −80° C. and fresh protease inhibitors added when thawed. 15 μg of nuclear lysates was usually used unless otherwise stated. SDS-PAGE was performed with 10% gels under standard conditions. Semi-dry electro-blotting of separated proteins was performed onto Immobilo-PSQ PVDF transfer membrane (Millipore) under standard conditions. Blocking was performed overnight at 4° C. on a rotator with 5% milk in PBS with 0.01% Tween-20 (Sigma-Aldrich) as the blocking agent.

Immunofluorescence staining was performed as follows. Culture medium was removed and cells washed thrice with PBS, then fixed with 4% paraformaldehyde (Sigma) for 30 minutes at room temperature. Excess paraformaldehyde was quenched with 50 mM NH₄Cl for 10 minutes. Fixed cells were permeabilized with 0.1% (v/v) Triton-X in PBS for 30 minutes. Blocking was performed using 1% BSA (w/v) in PBS for 30 minutes at room temperature, and staining performed with anti-HBs (sc-52411, 1:100, Santa-Cruz) in blocking buffer overnight at 4° C. in a humidified chamber. After washing thrice in PBS at room temperature for 10 minutes each, DyLight-488 conjugated secondary antibody (1:100, Thermo Scientific) was added at room temperature in the dark for 1 hour. Nuclei are stained with 500 ng/ml DAPI (Thermo Scientific) for 10 minutes before washing thrice in PBS and mounting.

Antibodies for Lamin B1 (Abcam) and PARP-1 (Santa-cruz) were added at room temperature in blocking reagent in concentrations as recommended, and incubated with the membrane on a rotator for 2 hours. Blots were washed thrice, 10 minutes each with 0.01% Tween-20 in PBS. HRP-conjugated secondary antibodies (Dako) were diluted 20000× in blocking reagent, and incubated with the blot for 1 hour at room temperature on a rotator. After 3 washes in 0.01% Tween in PBS for 10 minutes each, specific protein bands were detected with the SuperSignal West Femto Maximum Sensitivity Substrate (Thermo scientific) chemiluminscent substrate diluted 4 times in water.

### Example 6

### Materials and Methods—Electrophoretic Mobility Shift Assays (EMSA)

EMSA was performed using the LightShift Chemiluminescent kit (Thermo Scientific) with 2 μg HepG2 nuclear lysates and ing 5′-end biotinylated probes (1^{st} base) (TTGAGGCCTACTTCAAAGACTGTGTG). Biotinylated EBNA probes provided in the kit were used as negative control. Binding was performed at 37° C. for 45 minutes using a thermocycler in 20 μl reactions. The binding buffer was comprised of 10× binding buffer provided in the kit, 12.5% glycerol, 0.5 mM EDTA, 0.3 mg BSA, 0.05% NP-40 and 1 μg poly-dIdC. 1 μl antibodies were used when indicated. The monoclonal antibody against human PARP-1 was purchased from Santa-Cruz, while HNF4α specific antibody was from R&D Systems. Electrophoresis was performed at 4° C. on pre-run 4% native gels at 100V for 1.5 hours. Subsequent steps were performed as directed in the instruction manual.

### Example 7

### Materials and Methods—Streptavidin Pull-Down and MALDI-TOF/TOF

10 μl of Dynabeads® M-280 Streptavidin (Invitrogen) were washed as recommended. 1 μg of biotinylated probes (1^{st} base) was used for the binding reaction. 70 μg HepG2 nuclear lysates were used in 100 μl binding reaction with binding buffer identical to that used in EMSA. Binding was performed for 45 minutes on a bench-top thermo-mixer at 37° C. with gentle agitation at 300 rpm to keep the probe-bound beads in suspension. Washing was performed as suggested in manufacturer's instructions with EMSA binding buffer. Bound proteins were eluted by boiling protein-bound beads at 95° C. for 20 minutes in 20 μl 2× reducing SDS-PAGE loading buffer. SDS-PAGE was performed using all the eluate in 10% gels under standard conditions with fresh reagents, and the gel stained with fresh, filtered Coomassie overnight. After de-staining with fresh reagents, the gels were washed thrice in milli-Q water in newly-unwrapped sterilin plates for 10 minutes each, and gels excised with new scalpels for each band of interest. Excised gels were sent to the Protein and Proteomics Center in the National University of Singapore for MALDI-TOF/TOF analysis.

### Example 8

### Materials and Methods—cccDNA Assay

Total DNA was extracted in replicates of 3 from cultures in 6-well plates using the DNeasy Blood and Tissue kit (Qiagen) in the presence of RNase A (Qiagen). Quantitative real-time PCR was performed using the LightCycler® FastStart DNA Master^{PLUS} SYBR Green I (Roche) 1 in 10 μl reactions containing ing total DNA. cccDNA was detected using the following primers:

cccF: 5′GCACCTCTCTTTACGCGGTCTCC
cccR: 5′TGAAGCGAAGTGCACACGGACCG

The relative amount of cccDNA was normalized to the relative amount of transfected pcDNA3.1+ vector to account for differences in transfection efficiency and normalized to that of 6 h post-transfection. The primers for detection of pcDNA3.1+ are as follows:

pcDNA-F1: 5′TGGATAGCGGTTTGACTCACGGGG
pcDNA-R1: 5′ATTTGCGTCAATGGGGCGGAGTTG

Thermocycling parameters are as follows: 94° C. for 5 seconds, 56° C. for 10 seconds, 72° C. for 10 seconds and fluorescence acquired at 78° C. for 45 rounds.

### Example 9

### Materials and Methods—PARP-1 Activity Assays

PARP-1 enzymatic assays were performed in triplicates using the PARP Universal Colorimetric Assay Kit (R&D Systems). 1 μg of nuclear lysates were used for determination of relative endogenous PARP-1 activity in different cell lines. 5 μg of nuclear lysates was used to test if DNA sequence-specific binding could inhibit PARP-1 activity. To obtain the test DNA duplexes, equal amounts of 100 μM DNA oligomers (1^{st} base) were mixed and incubated at 95° C. for 5 minutes, then cooled to 37° C. for 5 minutes and further cooled to 24° C. for another 5 minutes in a thermocycler. 1 μl of cooled annealed products were added per well. Lysates were dissolved in PARP buffer with PMSF and protease inhibitor cocktail (Sigma-aldrich). Assays were carried out as recommended.

### Example 10

### Materials and Methods—Alkaline COMET Assays

1.5×10⁵ HepG2 cells were reverse transfected with 1.1 μl of Lipofectamine 2000 (Invitrogen) and 1 μg of plasmid in 24-well plates. 24 hours later, culture medium was removed and fresh medium with drugs were added at the following concentrations, 20 ng/ml aqueous bleomycin (Sigma), 50 nM etoposide in DMSO (Sigma-Aldrich) or 0.02% aqueous N-nitros-N-methylurea (Sigma). Cells were collected at time-points as indicated in text and alkaline COMET assays were performed using the CometAssay® kit as instructed (Trevigen). At least 50 comets per treatment were analyzed and scored with the TriTek ComentScore™ version 1.5 software.

### Example 11

### Materials and Methods—Cell Death and Apoptosis Assays

Cell death was observed at time-points indicated by staining for annexin V using Annexin V-Fluos (Roche). At indicated time-points, culture medium was removed after gentle shaking to remove dead cells if any Annexin V-Fluos was added to Hepes buffer as indicated, and added to cells in the dark. Staining was performed on ice in the dark for 10 minutes. Excess dye was removed and fresh culture medium gently added and analyzed immediately.

Apoptosis was assayed by detecting for caspase-3 and caspase-7 activity using Caspase-Glo® 3/7 Assay (Promega) as recommended. Replicates of 5 were performed in 96-well plates. 3×10⁴ HepG2 cells were seeded per well and reverse transfected with 200 ng of plasmids using 0.22 μl Lipofectamine 2000 (Invitrogen). Culture medium was then replaced with 100 μl fresh culture medium containing DNA damage inducers as indicated before lysis with equal volume of reagent. Luminescence was measured with the GloMax® Multi-Microplate Multimode Reader (Promega) 25 minutes after lysis.

### Example 12

### Results—Identification of Important Regulatory Sequence in HBV Core Promoter

The core promoter has been described to contain several elements [13, 14, 20, 21]. It is made up of the upper regulatory region (URR) as well as the basal core promoter (BCP), which binds the basal transcriptional machinery. Several transcription factors bind the URR, but none of them are readily targetable as therapeutic candidates [14-19, 22]. To identify host proteins that bind to the URR, 21 sequential 15 base-pair deletions were designed and tested for promoter activity. The extensive overlapping of deletions decreases the chance of identifying false-positive regions and enables pinpointing of the exact nucleotides required for the novel binder to bind. The effect of each deletion was tested in HepG2, which is supportive of HBV replication.

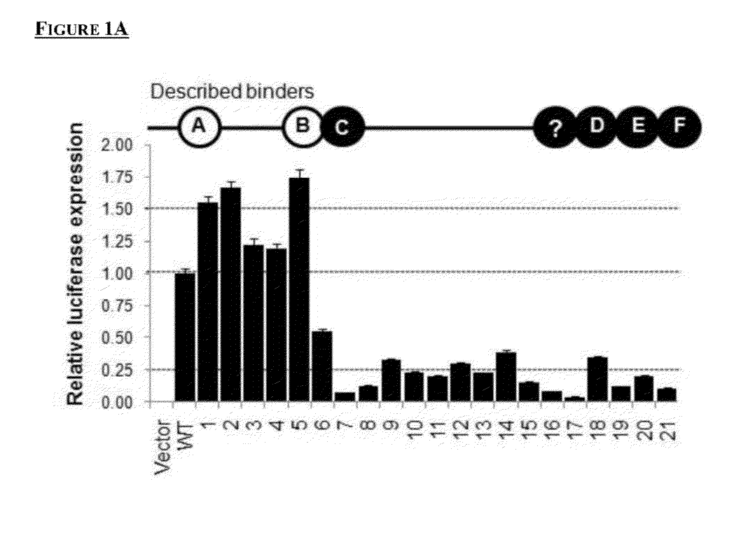

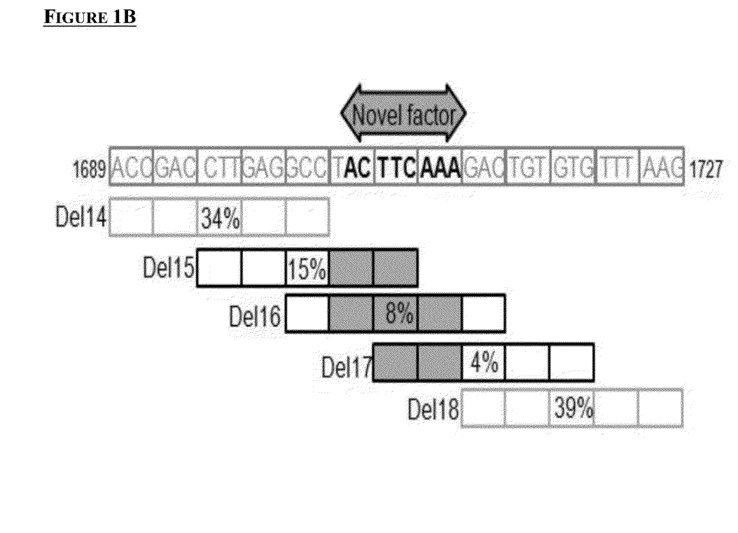

Consistent with the described roles of the regulatory elements in the core promoter in HBV supportive cell lines, deletions in the NRE (deletions 1 to 5) generally result in increased luciferase expression while deletions in enhancer II behaved otherwise (FIG. 1A). Deleted sequences that produce greater than 50% increase or greater than 75% decrease in luciferase expression were thought to be functionally important. These include deletions 1, 5, 7, 17, 19 and 21. Of these, deletion sequences 1 and 5 correspond to the binding sites of NREBP and FTF respectively, while deletion sequences 7, 19 and 21 correspond to the binding sites of C/EBP, HNF1 and SP-1 respectively. Deletion sequence 17 may bind to HNF1 or HNF3. However, since the overlapping deletion 18 was not classified to be functionally important, these candidates were eliminated. The sequence of deletion 17 was thus identified as a novel binding site for a regulator of HBV replication.

To pinpoint the binding sequence of the novel factor, the nucleotides corresponding to each deletion are indicated in FIG. 1B with the relative luciferase expression indicated as a percentage of that of the wild-type HBV core promoter. Since deletions of sequences 14 and 18 produce relatively mild phenotypes but deletion sequence 15 is affected to a great extent, the most important nucleotides must lie in the center of deletion sequence 16 and at the 5′ end of deletion sequence 17. This suggests that the sequence recognized by the novel factor correspond to “ACTTCAAA”.

### Example 13

### Results—PARP-1 Binds HBV Core Promoter in Sequence-Specific Manner

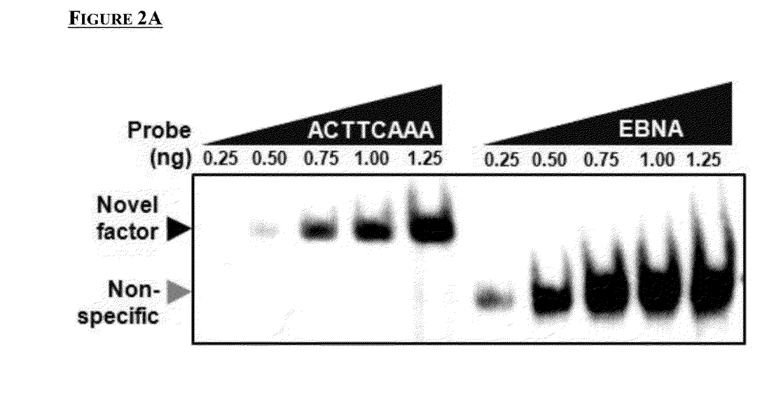

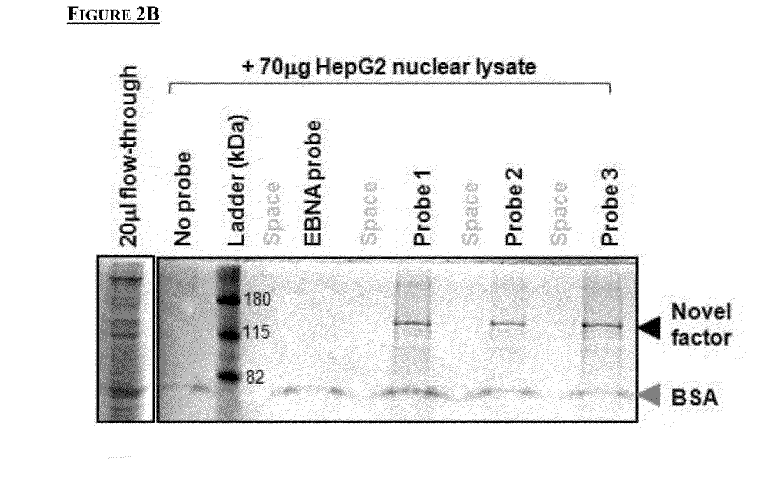

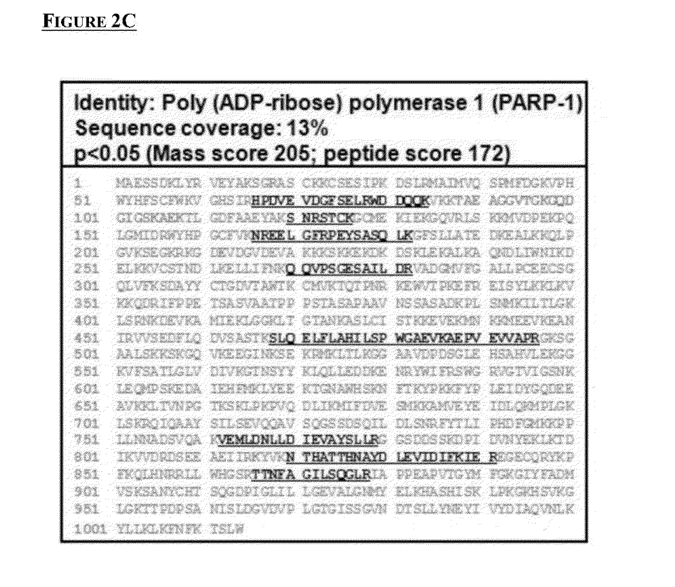

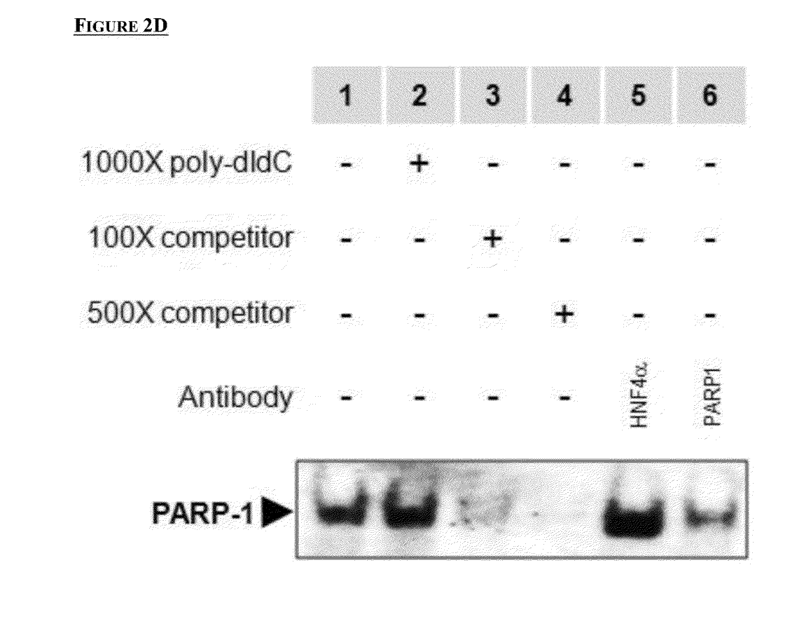

The “ACTTCAAA” sequence was biotinylated and used as bait to determine if it could bind the unknown novel factor specifically. As shown in FIG. 2A, the biotinylated sequence produced a novel band in a dose-dependent manner. This binding was sequence-specific, as the use of equivalent amounts of EBNA probe failed to produce a similar band.

The biotinylated sequence was subsequently used as bait in 3 independent streptavidin pull-down assays with HepG2 nuclear lysates. A specific binder of approximately 115 kDa was consistently identified, which expectedly could not bind to the EBNA probe (FIG. 2B). This protein was sent for MALDI-TOF/TOF analysis, and the results indicate poly (ADP-ribose) polymerase 1 (PARP-1) as the only candidate (FIG. 2C).

To prove that the novel factor of interest was indeed PARP-1, EMSA was performed with nuclear lysates from HepG2. To eliminate the possibility that PARP-1 bound non-specifically to the biotinylated probes by virtue of its ability to bind DNA strand breaks and blunt ends, 1000-fold excess of poly-dIdC on top of the huge excess of 1 μg poly-dIdC already present in the reaction was shown to be unable to remove the band of interest (FIG. 2D, lane 2). However, a much smaller amount of 100-fold excess of non-biotinylated probe was sufficient to do so (FIG. 2D lane 3). PARP-1 thus binds the probe in a sequence-specific manner. Furthermore, an antibody specific for the DNA-binding domain of PARP-1 was able to diminishing the PARP-1 specific band (FIG. 2D lane 6). This loss of intensity could not be obtained with non-specific antibodies such as those for HNF4α (FIG. 2D lane 5), indicating that PARP-1 binds the “ACTTCAAA” sequence hence HBV core promoter specifically. The effects on the core promoter activity observed with the deletion of site 17 were therefore as predicted, not due to HNF 1 or HNF3.

### Example 14

### Results—PARP-1 Modulates HBV Replication

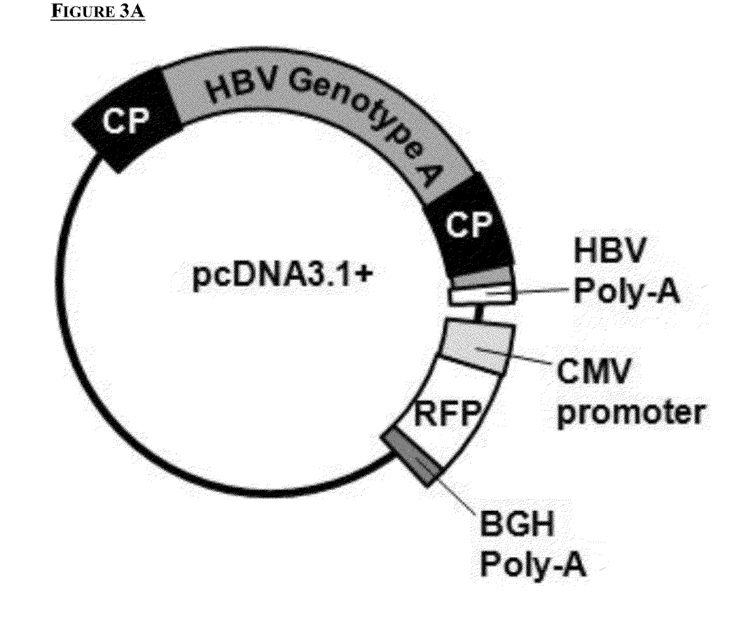

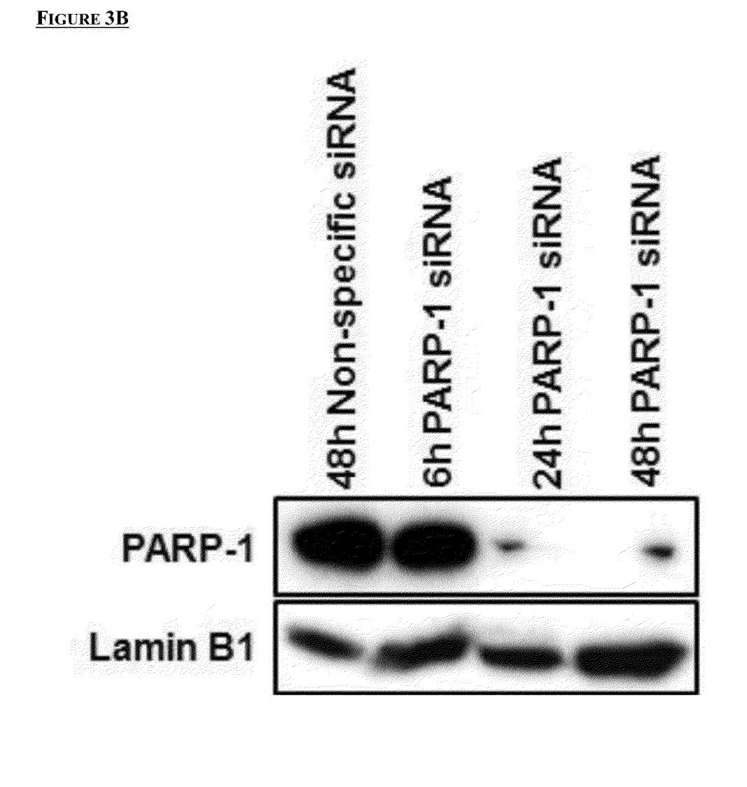

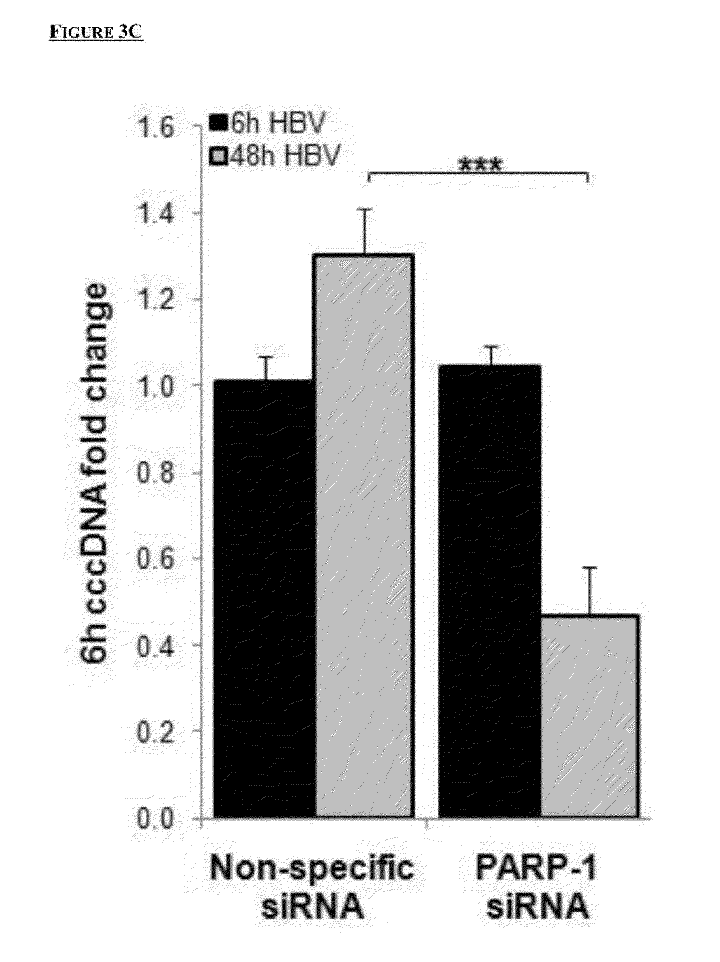

To test if PARP-1 was indeed a positive regulator of HBV replication, a replicative HBV construct was cloned and inserted upstream of the CMV promoter so that pgRNA transcription depended only on the HBV core promoter (FIG. 3A). The CMV promoter was used to drive RFP (red fluorescent protein) expression, which served as an indicator for transfection efficiency.

The construct was transfected into HepG2 cells 24 hours after PARP-1 specific knock-down, by which time PARP-1 protein expression was significantly reduced (FIG. 3B). PARP-1 knock-down had little effect on cell survival, consistent with its redundancy in rodent knock-out models [23-25]. Transfection efficiency of the construct was also not affected by the use of PARP-1 specific siRNA or non-specific siRNA. The relative amount of cccDNA synthesized from pgRNA was compared between PARP-1 specific and non-specific knock-down. As shown in FIG. 3C, cells with non-specific knock-down increased cccDNA production as a result of pgRNA synthesis whereas in PARP-1 specific knock-down, the increase in cccDNA production was not observed. These results indicate that the loss of PARP-1 is detrimental to HBV replication. Hence PARP-1 binding to its motif in the HBV core promoter regulates pgRNA transcription for the efficient replication of HBV.

PARP-1 enzymatic activity is required for its DNA repair function. To determine if it is also required for PARP-1 motif dependent transcription, the effect of enzymatic inhibition by 3-aminobenzamide (3-AB) was investigated.

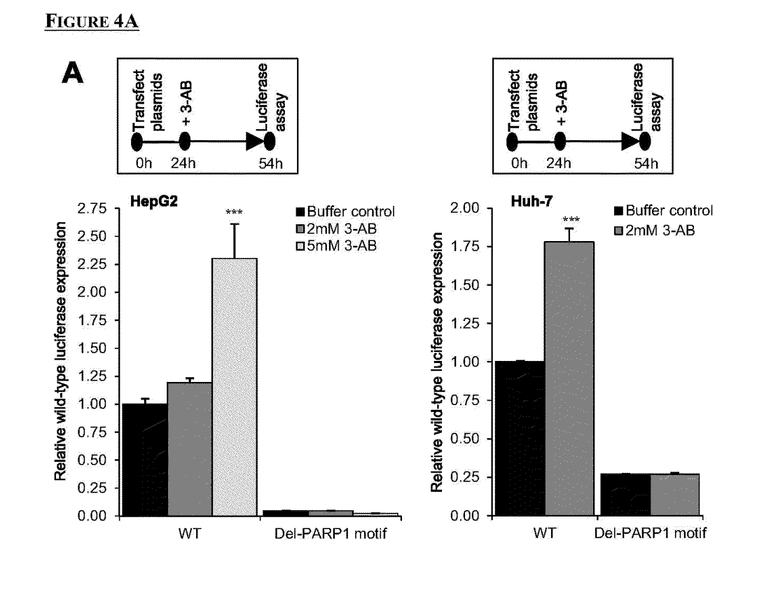

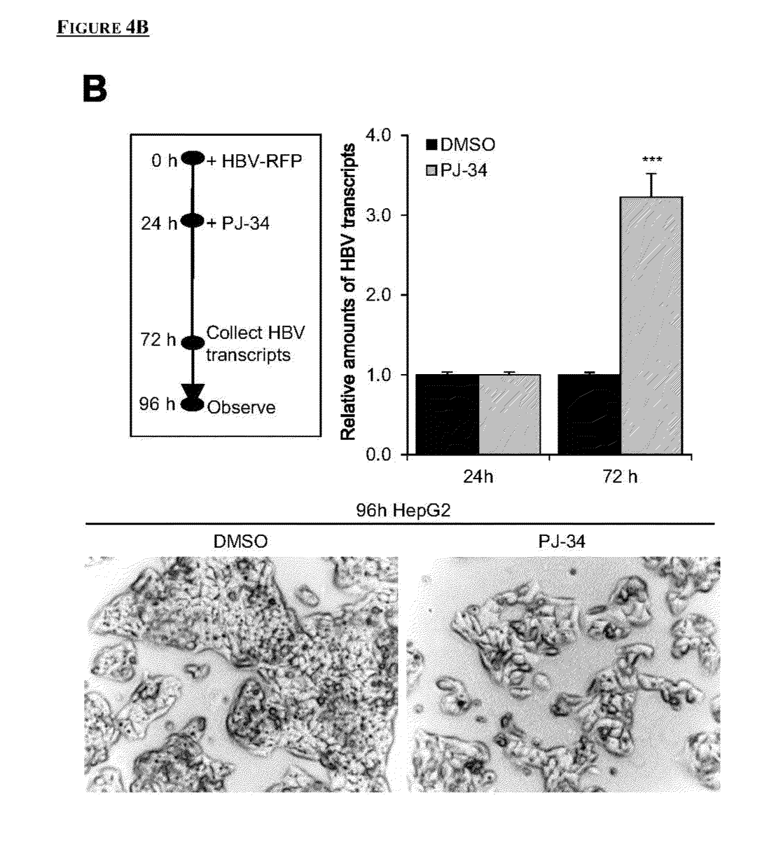

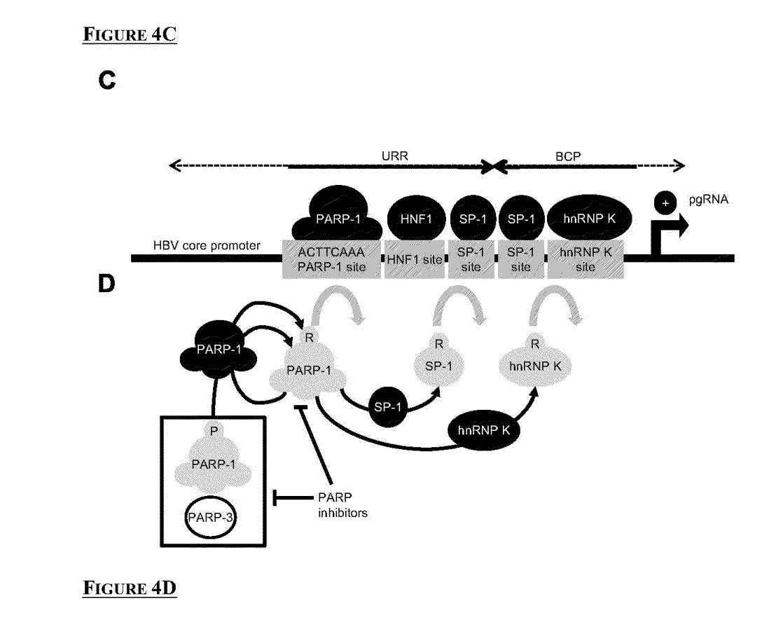

HepG2 and Huh-7 cells were transfected with the wild-type HBV core promoter and PARP-1 was inhibited 24 hours after transfection. In contrast to its DNA repair function, PARP-1 inhibition increased transcription at the HBV core promoter in a dose-dependent manner in HepG2 cells (FIG. 4A). Since deletion of the PARP-1 motif did not result in a dose-dependent increase in luciferase expression, this result indicates that the specific inhibition of PARP-1 enhances transcription. Similarly, Huh-7 cells also display increased luciferase expression with 3-AB treatment.

As transcription at the HBV core promoter is critical for HBV replication, the effect of enhanced transcription by PARP-1 inhibition on the amount of HBV transcripts was investigated. HepG2 cells transfected with HBV-RFP was treated with PJ-34 (15 μM) 24 hours after transfection, and this resulted in more than 3-fold increase in the relative amounts of HBV transcripts 48 hours later (FIG. 4B). Furthermore, 72 hours after PARP inhibition, the relative percentage of viable cells that were HBV-transfected was significantly higher than cells treated with DMSO control.

Thus, PARP-1 inhibition enhances HBV replication by enabling increased transcription at the HBV core promoter in a PARP-1 motif dependent manner.

### Example 15

### Discussion: Examples 1 to 14

This study shows that PARP-1 is a transcriptional activator important for HBV replication. Targeting PARP-1 may prove to be a useful strategy for the treatment of HBV infection. However, conventional drugs act by inhibiting the enzymatic activity of PARP-1, which could lead to enhanced HBV replication. This is because PARP-1 enzymatic inhibition prevents auto-ADP-polymerization on PARP-1, to which 90% of cellular ADP-ribose polymers are added, increasing its net negative charge hence preventing it from binding DNA [26, 27]. Such a phenomenon has been observed in the transcription of vimentin and transcriptional activity at the human T-cell lleukemia virus type I tax-responsive element [28, 29]. While the PARP-1 enzyme is known to be activated by binding to DNA strand breaks, enzymatic activation need not always be so [30-32]. It may also be brought about by PARP-1 modifications including phosphorylation, interaction with other proteins such as phosphorylated ERK2 and even by ADP-ribosylation mediated by other PARP family members. In other words, in the absence of DNA damage, a fraction of PARP-1 exists as its ADP-ribosylated form. Enzymatic inhibition with conventional inhibitors without DNA damage induction would therefore be effective to increase the pool of unmodified PARP-1, increasing transcriptional activation hence HBV replication.

Interestingly, PARP-1 ADP-ribosylates both SP-1 and hnRNP K that bind within the vicinity of its binding site in the HBV core promoter [14, 26, 38, 39] (FIG. 4C). The PARP-1 enzyme can also modify other transcription factors, including nuclear receptors such as HNF4α and COUP-TF that are known to mediate pgRNA synthesis [39-42]. By conferring an increase in net negative charge for DNA repulsion, enzymatically activated PARP-1 may rapidly inactivate multiple transcription factors required for HBV replication (FIG. 4D). As such, targeting PARP-1 alone could be an efficient means of controlling hepatitis B, as was indicated by the reduction of cccDNA synthesis in PARP-1 knock-down cells (FIG. 3C).

While PARP-1 enzymatic inhibition might increase transcription at the HBV core promoter, this phenomenon seems at odds with previous findings that HBV replication depended on ATR activation, as the latter is known to depend on PARP-1 enzymatic activity [43]. The role of ATR in HBV replication has not been determined. Considering that the active PARP-1 enzyme is involved in re-starting stalled replication forks and is also involved in the recruitment of ATR to activate Chk1 for cell cycle arrest in the DNA damage response to unreplicated DNA [44, 45], it is conceivable that PARP-1 enzymatic activity is required to synthesize cccDNA from rcDNA, the partially double-stranded DNA made from pgRNA of HBV that is encapsidated and occasionally recycled to maintain the nuclear cccDNA pool [6-9, 12]. In such case, PARP-1 enzymatic activity is only required in the early stages of HBV infection, when cccDNA is synthesized from rcDNA, and when the nuclear pool of cccDNA needs to be replenished.

A fine balance of PARP-1 enzymatic activity needs to be maintained for efficient HBV replication. High or normal PARP-1 enzymatic activity completes cccDNA synthesis from rcDNA carried by infectious particles. This need not depend upon DNA sequence-specific binding. The newly made cccDNA forms the template to initiate pgRNA synthesis, which is dependent on PARP-1 sequence-specific binding on the core promoter, hence requiring PARP-1 to be an inactive enzyme. Large amounts of virions may then be produced from pgRNA, hence effective HBV replication. Suppression of PARP-1 enzymatic activity to a basal minimum allows large amounts of pgRNA to be transcribed, while ensuring that minute amounts of rcDNA made from pgRNA can be recycled to maintain the nuclear cccDNA pool for sustenance of HBV replication.

Taken together with the reduction of HBV replication in PARP-1 knock-down cells, the proposed model of PARP-1 dependent HBV replication predicts that PARP-1 sequestration instead of enzymatic activity modulation would be more feasible for the treatment of hepatitis B. Since no drugs are known to have PARP-1 sequestration activity, studying sequence-specific recognition may be a good starting point for the development of a novel class of PARP-1 activity modulators. This may also prove useful in other PARP-1 dependent diseases such as inflammatory disorders [24, 36, 46] and help reduce contraindications for multiple conditions such as cancer, inflammation and HBV infection.

### Examples Part 2

### PARP-1 Inhibition by Motif Recognition

### Example 16

### PARP-1 Recognition Motif

PARP-1 binds the “ACTTCAAA” sequence of the HBV core promoter to regulate HBV replication (FIG. 1). To determine the importance of this sequence in PARP-1 dependent transcription, the effect of mutating every nucleotide was investigated.

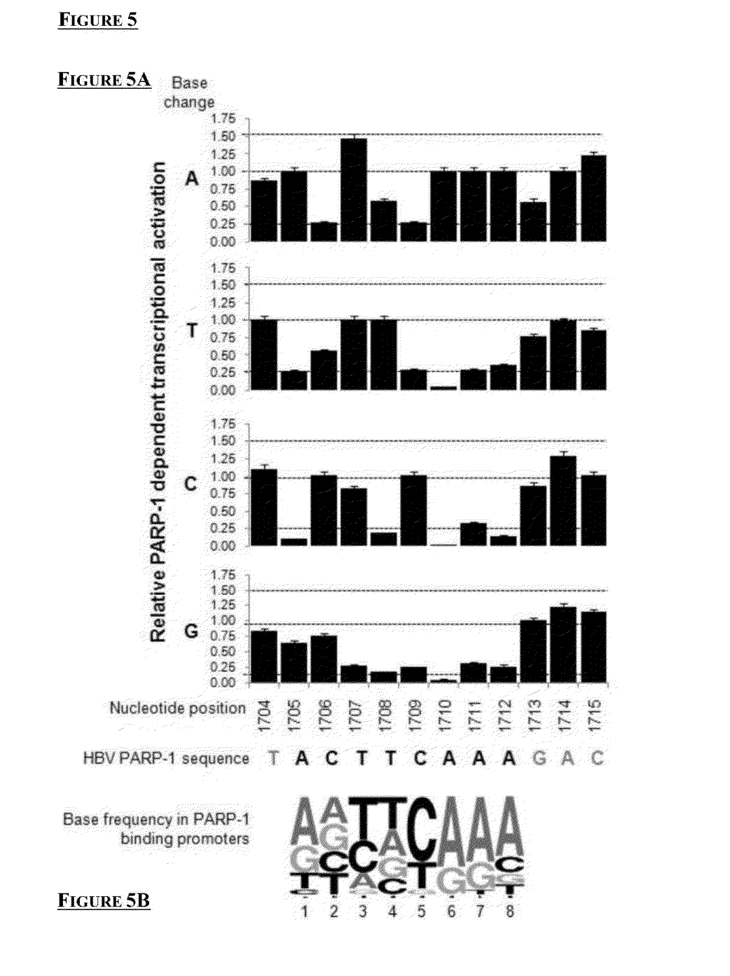

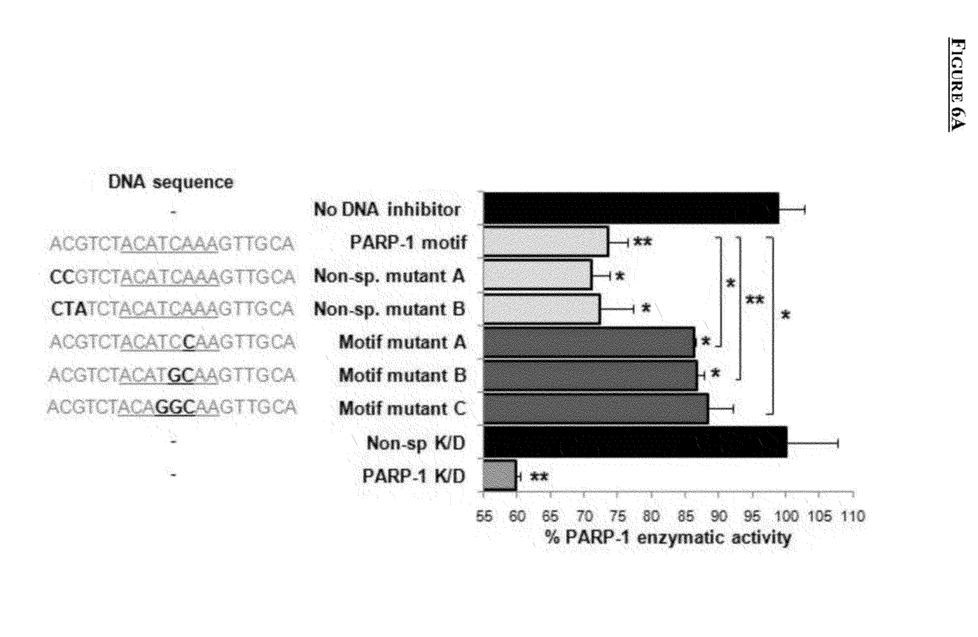

As summarized in FIG. 5A, mutation of flanking nucleotides T1704, G1713, A1714 and C1715 had little effect on PARP-1 dependent transcription whereas at least 1 mutation on each position of the “ACTTCAAA” sequence resulted in the loss of greater than 75% PARP-1 dependent transcriptional activation. In particular, A1710 is the most important nucleotide of the sequence as all base changes reduced PARP-1 dependent transcription to less than 10% of wild-type. The T1707A mutation was the only mutation that increased luciferase expression by nearly 50%, suggesting that the “ACATCAAA” sequence is the optimal recognition sequence for PARP-1 dependent transcription. This is the first report of a consensus motif that binds specifically to PARP-1.

The “ACATCAAA” optimal PARP-1 dependent transcriptional activation motif identified must bear resemblance to known PARP-1 binding sequences. As a consensus PARP-1 binding sequence has not been described, multiple alignments of known PARP-1 binding sequences were used to obtain a frequency plot (FIG. 5B) [28, 29, 66, 68-79]. As shown, the HBV PARP-1 binding sequence bears great resemblance to the pre-dominant “ANTNCAAA” sequence obtained from the frequency plot, where “N” refers to a position with no specific base preference. Thus, the HBV core promoter contains a conserved PARP-1 binding sequence, enabling PARP-1 dependent pgRNA synthesis for HBV replication.

### Example 17

### PARP-1 Enzymatic Inhibition by Motif Recognition

PARP-1 binding to DNA strand breaks activates its catalytic activity to result in auto-ADP-ribosylation [55]. Whether PARP-1 motif recognition in sequence-dependent transcriptional regulation also results in the activation of the PARP-1 enzyme is however unknown.

To test this, short double-stranded DNA duplexes containing the “ACATCAAA” optimal PARP-1 dependent transcription motif was tested with nuclear lysates extracted from HepG2. Interestingly, the addition of DNA duplexes containing the PARP-1 motif reduced PARP-1 enzymatic activity when compared to lysates with no DNA duplex added (FIG. 6A). This phenomenon was motif sequence-dependent, as disruption of the optimal recognition sequence within the core of the 8-nucleotide sequence by increasing single-base mutations gradually alleviated PARP-1 enzymatic inhibition. Mutation of flanking nucleotides (Non-sp. mutant) however had little effect on motif-dependent PARP-1 inhibition. Taken together, these results suggest that, in contrast to the binding of DNA strand-breaks, sequence-specific binding of PARP-1 to its recognition motif inhibits PARP-1 enzymatic activity. How this may be achieved is however unknown.

PARP-1 is a large protein with at least 6 functional domains (FIG. 6B). While recognition of DNA strand breaks is dependent on zinc finger 1 and zinc finger 2, the domains required for PARP-1 motif recognition remain unresolved.

To address this, single amino substitutions on highly conserved amino acid residues within each functional domain were tested for its ability to activate transcription at the HBV core promoter when over-expressed in HepG2 cells.

Interestingly, 5 single amino acid substitutions across 3 functional domains resulted in the loss of ability to trans-activate the HBV core promoter (FIG. 6C). These domains correspond to zinc finger 1, zinc finger 2 as well as the BRCT domain. This result indicates that several domains of PARP-1 act together for the recognition of the PARP-1 motif.

However, as none of the amino acid substitutions in the catalytic domain disrupted the transcriptional activation capacity of PARP-1, this suggests that the PARP-1 motif is unlikely to compete with other PARP inhibitors for the catalytic domain's active site.

Thus, the PARP-1 motif may be used as an allosteric inhibitor of PARP-1 ADP-ribosylation function.

### Example 18

### PARP-1 Inhibition by Motif Recognition Sensitizes Cancer Cells to DNA Damage-Induced Cell Death

To date, all commercial PARP-1 inhibitors act by inhibiting the catalytic domain of the enzyme [33, 37]. PARP-1 sequestration hence enzymatic inhibition by motif recognition is thus a novel means of PARP-1 activity modulation that can be further developed for therapeutic purposes.

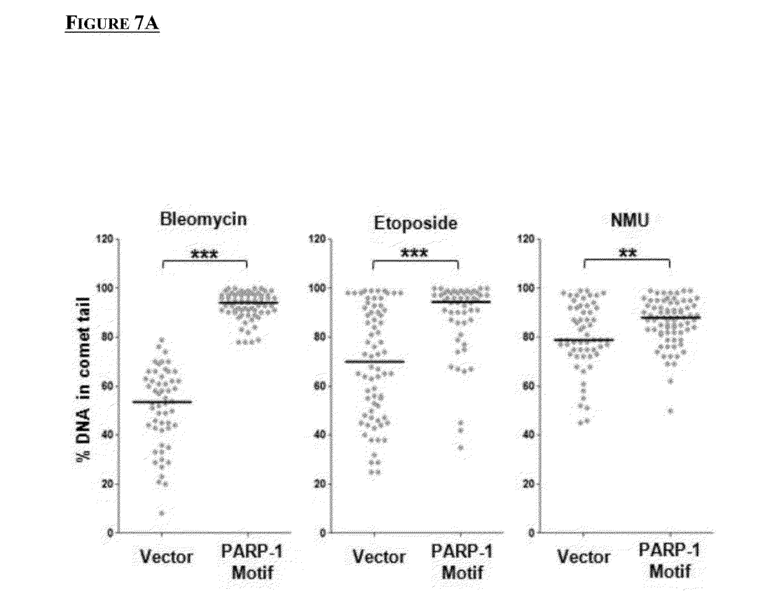

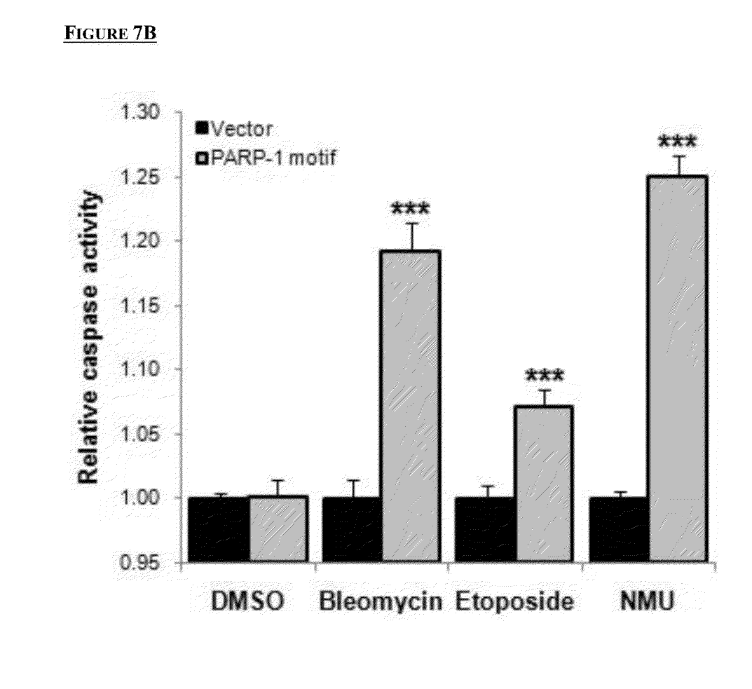

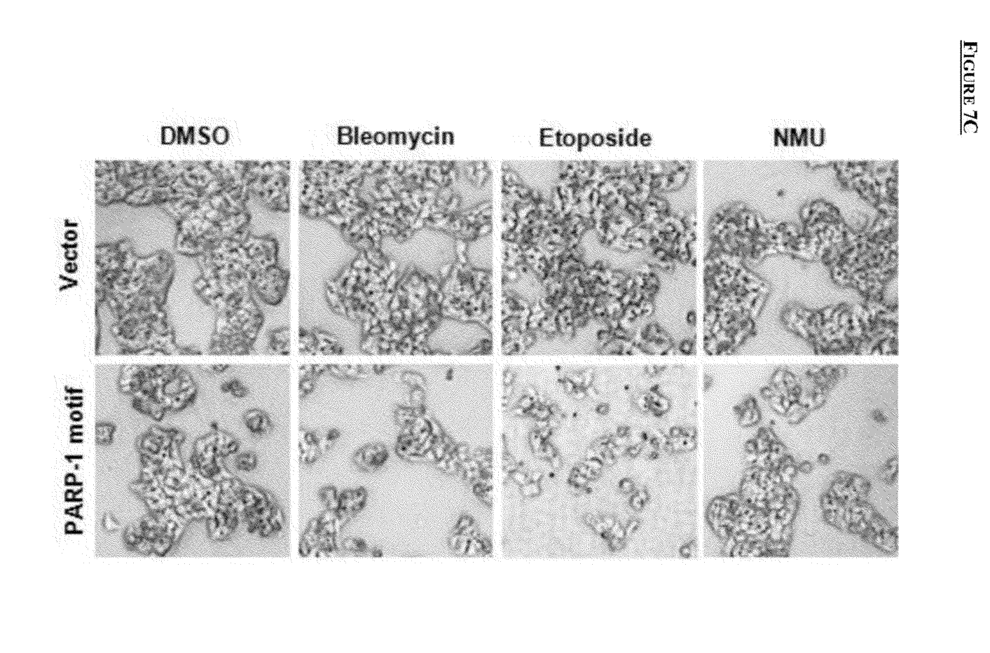

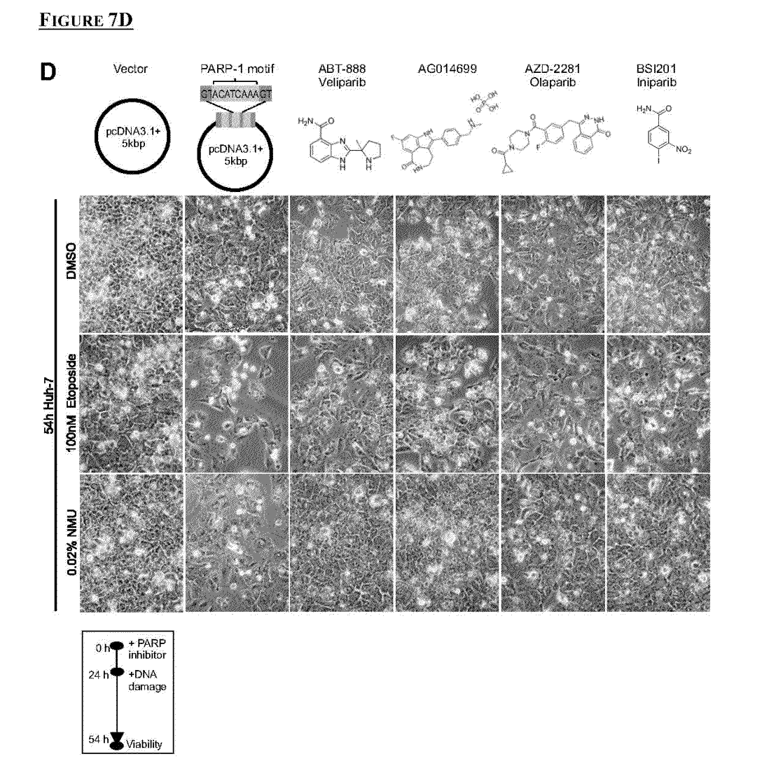

To show that the PARP-1 motif alone can impair PARP-1 enzymatic activity to bring about reduced DNA damage repair, 3 tandem repeats of the sequence “ACATCAAA” was inserted into the pcDNA3.1+ vector and reverse transfected into HepG2 liver cancer cells to increase transfection efficiency hence increased homogeneity in cell population. 24 hours after transfection, DNA damage-inducing drugs were added, and the resultant DNA damage was determined by alkaline COMET assay 18 hours later. As shown in FIG. 7A, treatment with the DNA double-strand break inducer bleomycin (20 ng/ml) increased DNA damage in a greater proportion of cells transfected with the PARP-1 motif. The same phenomenon was observed with the DNA alkylating agent N-nitroso-N-methyl-urea (0.02% NMU) and the DNA strand-break inducer etoposide (50 nM). These data suggest that DNA single and double-strand break repair, as well as base excision repair are impaired in cells transfected with the PARP-1 motif Since transfection of control plasmids alone is unlikely to bring about the reduction in PARP-1 activity, the effect must be due to the PARP-1 motif sequestering and inhibiting nuclear PARP-1. Therefore, PARP-1-specific binding can impair PARP-1 DNA repair activity in vivo.

Persistent irreparable DNA damage leads to cell death. This is a strategy commonly used in cancer therapy. To further prove that the PARP-1 motif can be used as a novel means to enhance irreparable DNA damage, the relative extent of apoptosis was measured by caspase activation at an early time-point of 18 hours after DNA damage induction. Consistent with the increased severity of DNA damage in a larger proportion of cancer cells, FIG. 7B shows that apoptosis was increased in cells transfected with the PARP-1 motif when compared to vector controls for all drug treatments.

This increased cell death was also reflected by enhanced annexin V staining in HepG2 cells transfected with the PARP-1 motif when compared to vector controls (FIG. 7C).

To demonstrate the feasibility of the PARP-1 motif as a novel agent for enhanced cytotoxicity when used in conjunction with DNA damaging agents, Huh-7 cells transfected with the PARP-1 motif were compared to cells treated with clinical PARP inhibitors (10 μM) and DNA damage was induced 24 hours later with etoposide (100 nM) or 0.02% NMU.

While all PARP inhibitors tested resulted in reduced cell viability when compared to DMSO controls, cells transfected with the PARP-1 motif displayed significantly reduced viability than clinical PARP inhibitors (FIG. 7D).

Taken together with the enhanced viability of cells transfected with the PARP-1 motif in the absence of DNA damage induction (FIG. 7D), these results indicate that PARP-1 enzymatic inhibition by the PARP-1 motif is an alternative to specifically reduce DNA damage repair hence associated cell death.

### Example 19

### HBV Replication Impairs PARP-1 Dependent DNA Repair

The HBV core promoter contains a well-conserved PARP-1 motif Since PARP-1 enzymatic activity hence DNA repair may be impaired by sequence-specific binding, this suggests that by increasing the nuclear cccDNA concentration hence PARP-1 binding sites, HBV replication may alter the host cell's ability to repair DNA.

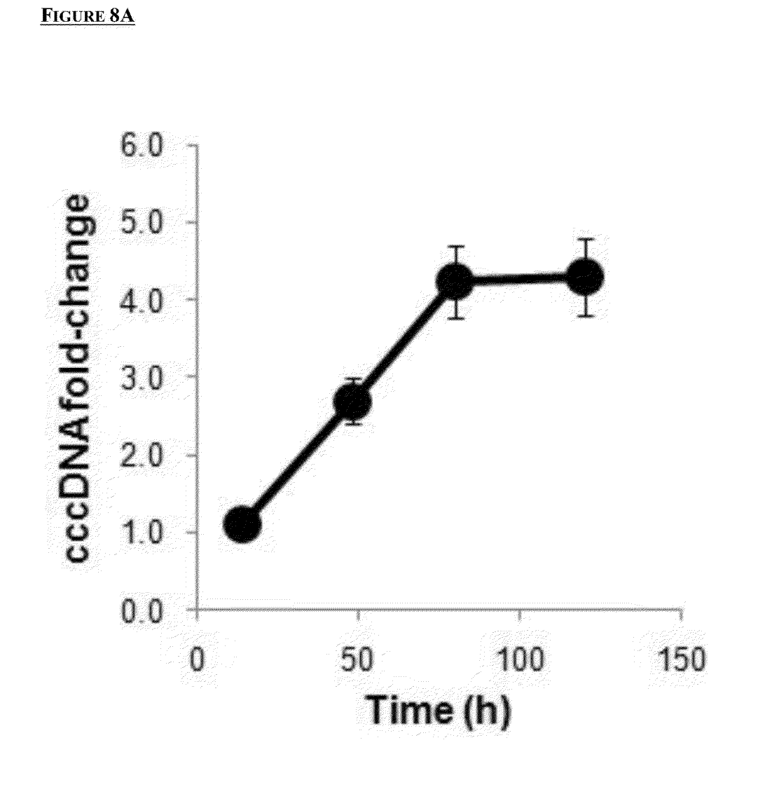

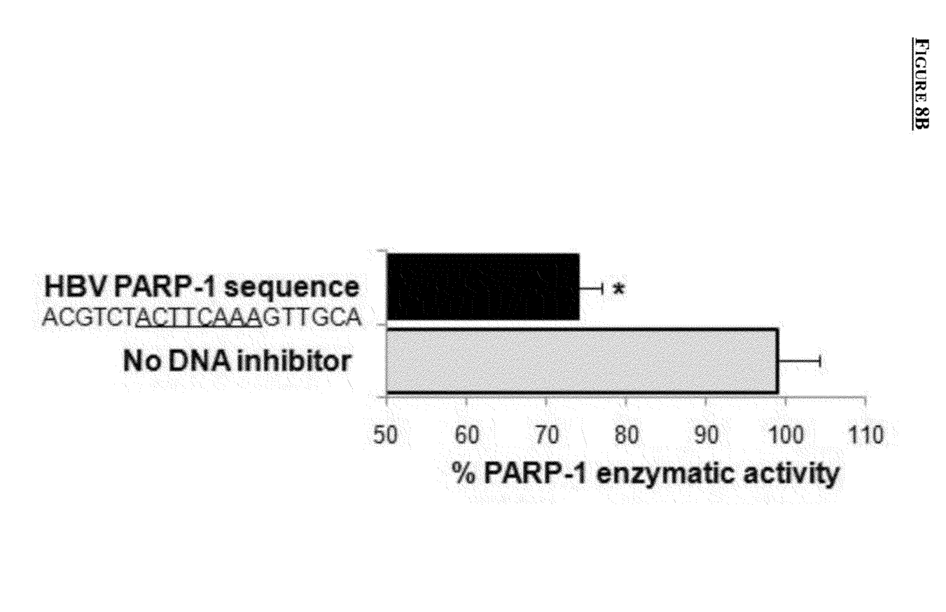

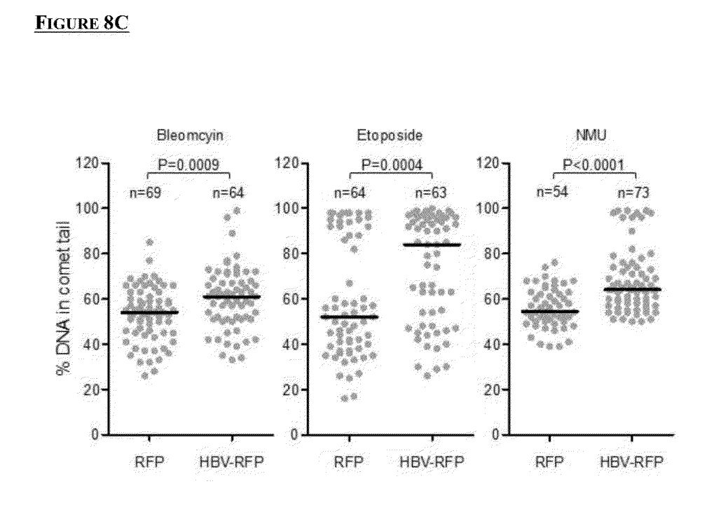

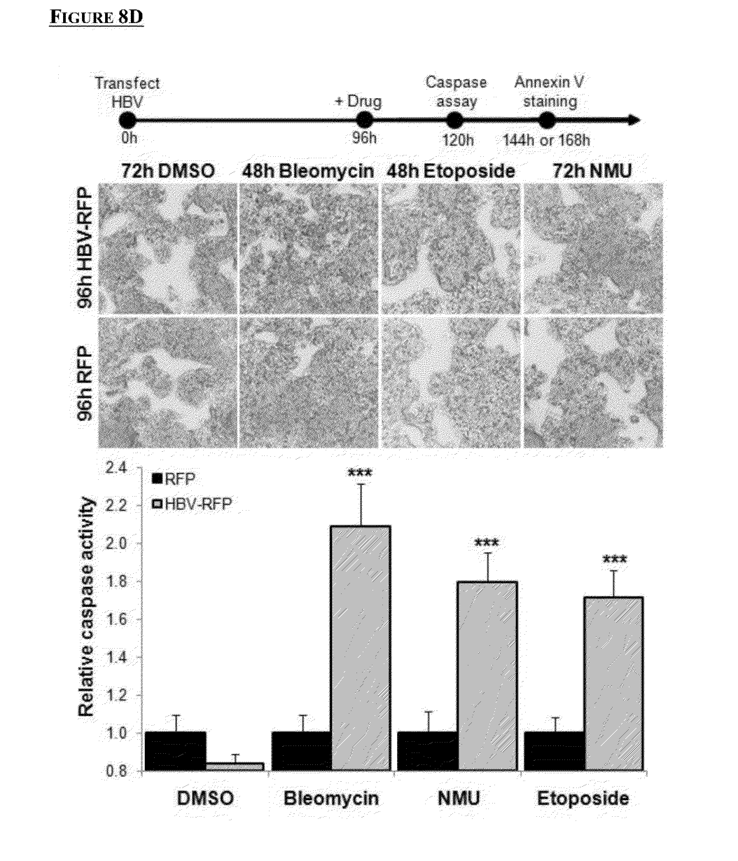

HepG2 cells were reverse transfected and cccDNA was shown to increase to maximum at around 96 hours later (FIG. 8A). Incubation of the HBV PARP-1 recognition sequence was also shown to have PARP-1 enzymatic inhibition activity (FIG. 8B). Thus any impairment of PARP-1 dependent DNA repair by HBV replication would be readily identifiable 96 hours post-transfection. DNA damage inducers were added at the indicated time-point, and alkaline COMET assays performed 24 hours later revealed increased DNA damage with bleomycin, etoposide and NMU in greater proportion of cells that have been transfected with the HBV-RFP construct (FIG. 8C). HBV replication per se did not result in cell death, as DMSO-treated cells did not stain positive for phosphatidylserine (FIG. 8C). Therefore, PARP-1 dependent DNA repair involving base-excision repair and strand breaks repair are impaired in HBV replication as a result of PARP-1 sequestration and enzymatic inhibition as predicted. This can lead to the accumulation of irreparable DNA damage, as indicated by increased DNA damage induced cell death shown by increased annexin V staining for phosphatidylserine and increased caspase activity (FIG. 8D). Therefore, HBV replication in host cells impairs PARP-1 dependent DNA repair, potentially leading to the gradual accumulation of DNA insults hence the development of HCC.

### Example 20

### PARP-1 Motif Specifically Inhibits PARP-1 in a Sequence-Dependent Manner without Enhancing HBV Replication

To demonstrate that the increased DNA damage and associated cytotoxicity brought about by the PARP-1 motif was mediated specifically by the inhibition of PARP-1, the effects of PARP-1 over-expression in cells transfected with the PARP-1 motif was investigated.

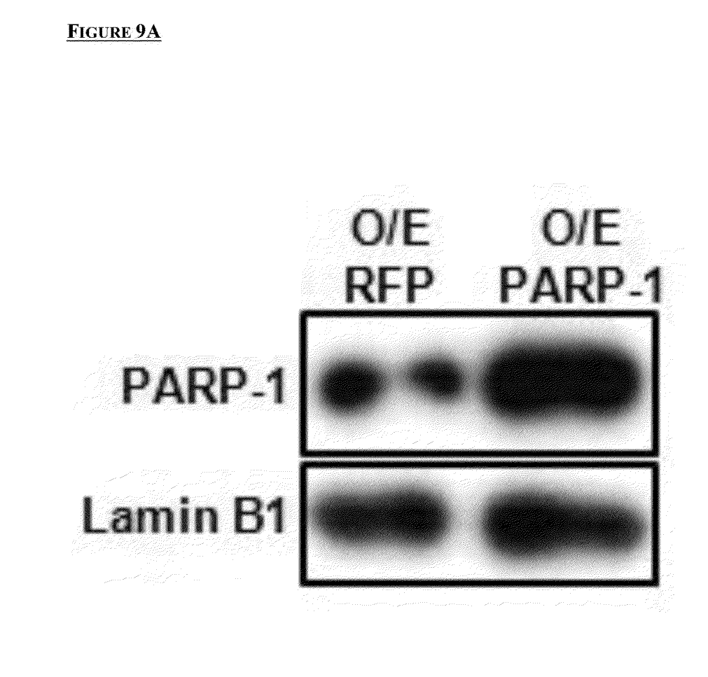

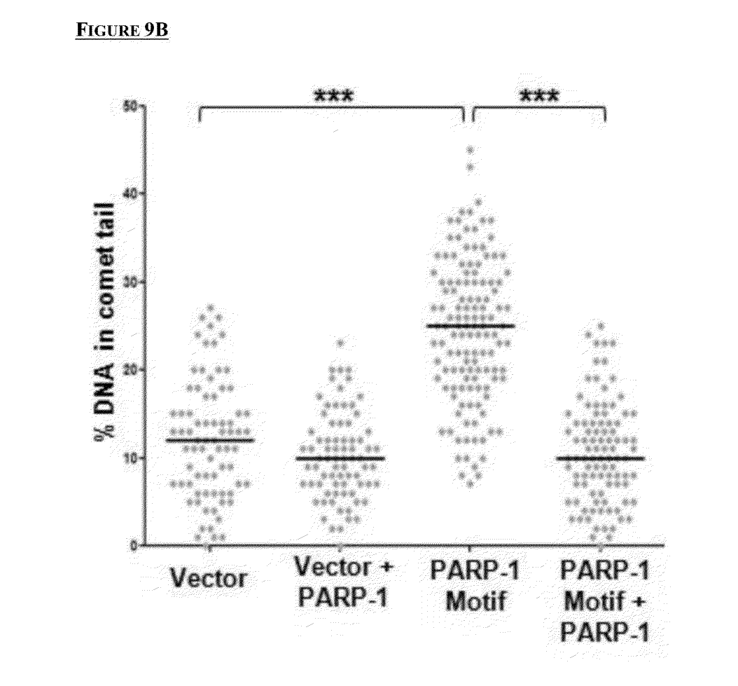

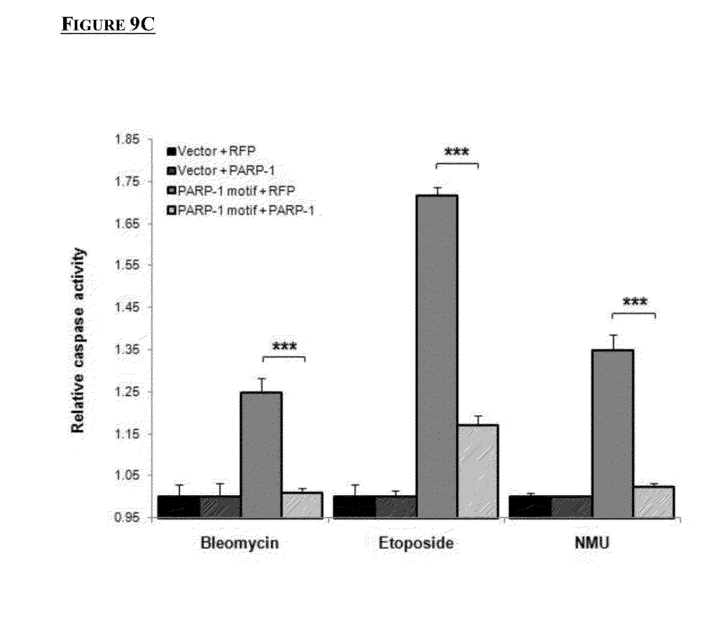

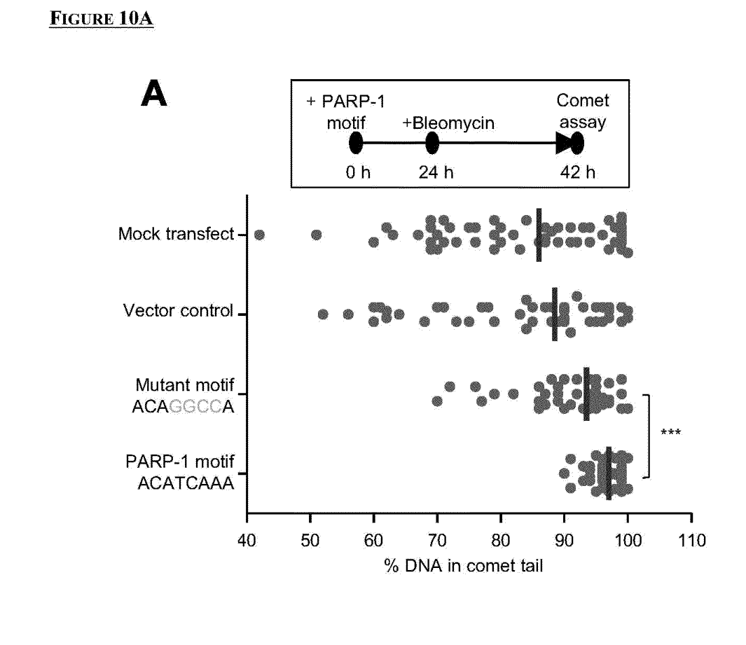

Wild-type PARP-1 was cloned into an expression vector and shown in FIG. 9A to be over-expressed in the nucleus 48 hours after transfection. As expected, reduced DNA damage was observed by alkaline COMET assay with increased PARP-1 expression in Huh-7 cells transfected for 48 hours with the PARP-1 motif that have been exposed to 24 hours of 0.01% NMU (FIG. 9B).

Furthermore, apoptosis was reduced in HepG2 cells transfected with the PARP-1 motif as shown by a reduction in caspase activity 48 hours post-transfection and after 24 hours of treatment with the DNA damage inducers bleomycin (10 ng/ml), etoposide (100 nM) or NMU (0.02%) in the presence of increased amounts of nuclear wild-type PARP-1 (FIG. 9C).

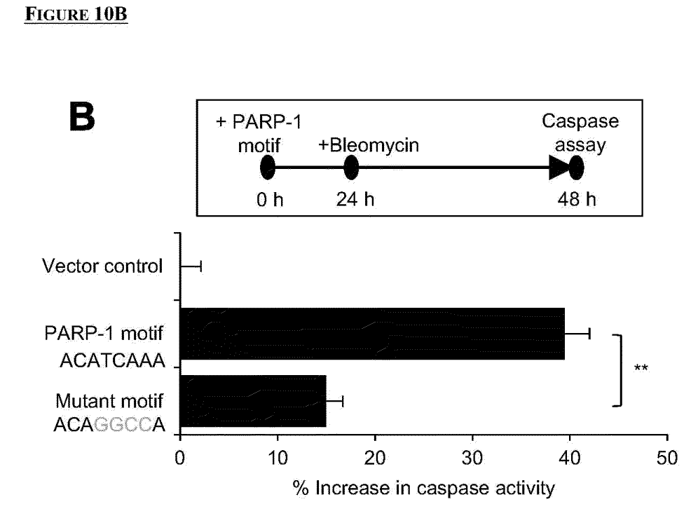

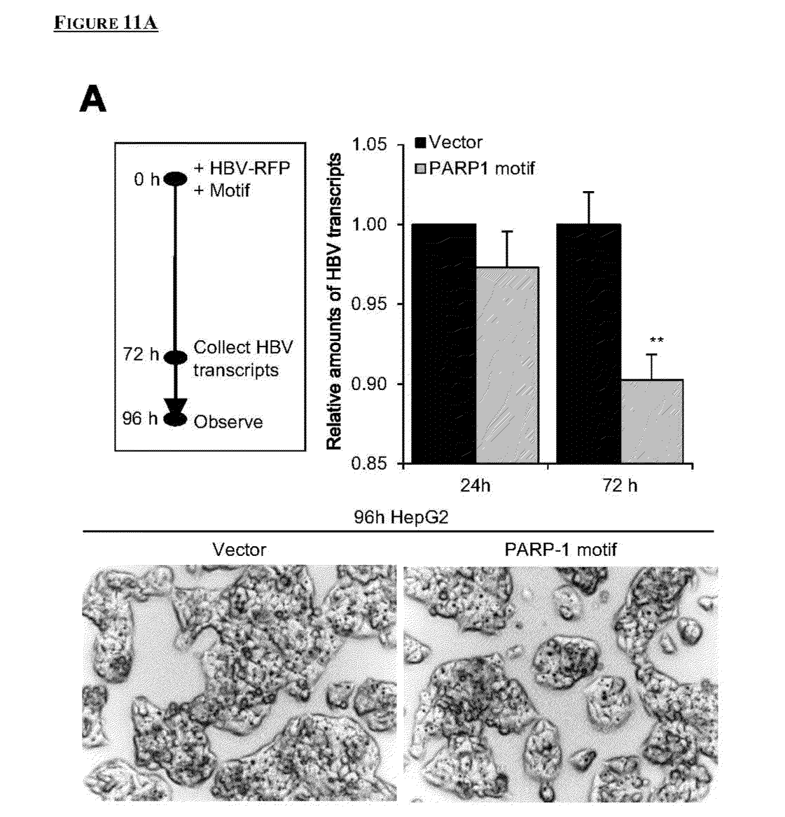

To ascertain that the impaired DNA repair capacity of PARP-1 was due to the octamer sequence of the PARP-1 motif, the effect of mutating core nucleotides 4-7 on enhanced apoptosis as a result of irreparable DNA damage was investigated. When transfected into HepG2 cells, constructs bearing 3 tandem repeats of the PARP-1 motif sequence “ACATCAAA” induced significant DNA damage in cells treated with bleomycin (20 ng/ml). However, cells transfected with constructs bearing the mutant “ACAGGCCA” motif had much lower amounts of damaged DNA (FIG. 10A). This resulted in less caspase activity hence apoptosis associated with irreparable DNA damage (FIG. 10B). Taken together with the results obtained from FIG. 9, the PARP-1 motif is a DNA specific sequence that acts to inhibit PARP-1. Therefore, the sensitization of cells to induced DNA damage and associated cytotoxicity induced by the PARP-1 motif in FIG. 7 and by HBV in FIG. 8 are specific to the impairment of PARP-1 dependent DNA repair.

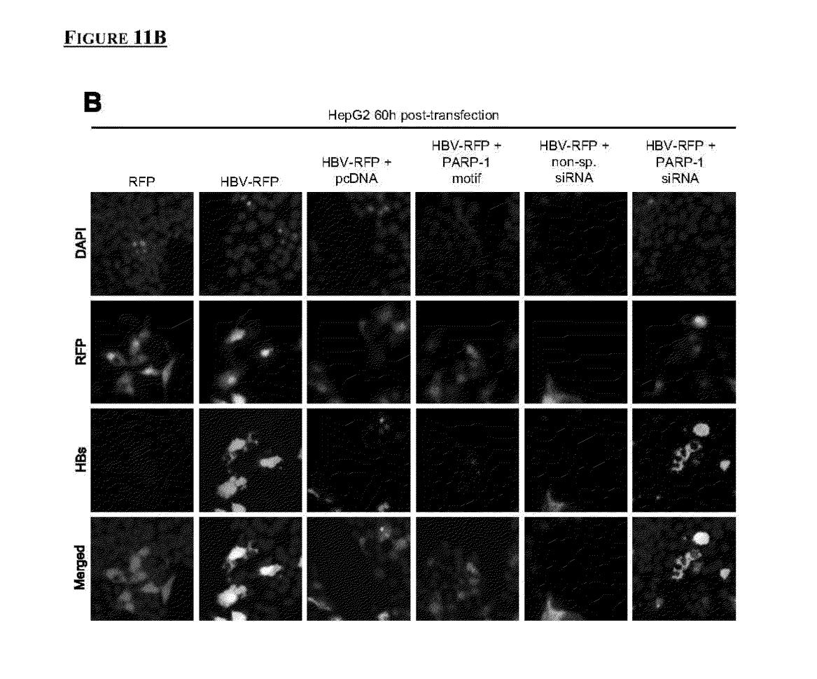

It was shown in FIG. 4 that PARP-1 inhibitors acting via the PARP-1 catalytic domain can enhance HBV replication. Given that the PARP-1 motif does not act on the catalytic domain (FIG. 6C), and that it is able to compete with the HBV core promoter for PARP-1 binding, the interesting interaction between endogenous PARP-1 and the PARP-1 motif on HBV replication was investigated. HepG2 cells were co-transfected with 1 μg of HBV-RFP and 500 ng of PARP-1 motif, and the relative amount of HBV transcripts generated compared with HBV-RFP transfected cells treated with empty vectors. FIG. 11A shows a reduction in the amount of HBV transcripts in cells 72 hours after co-transfection with the PARP-1 motif, suggesting that the PARP-1 motif can inhibit HBV replication. This is further supported by the reduction in HBs expression, which was only achievable with the specific knock-down of PARP-1 expression by specific siRNA (FIG. 11B). Therefore, the PARP-1 motif inhibits PARP-1 enzymatic activity specifically without enhancing HBV replication, eliminating the potential of aggravating hepatitis B.

### Example 21

### Discussion: Examples 16 to 20

PARP-1 can regulate transcription in many ways. Besides regulating chromatin compaction, PARP-1 has been shown to bind promoters in a sequence-dependent manner [28, 29, 41, 66-71, 73, 74, 76, 78, 79]. However, no PARP-1 sequence-specific binding consensus has been identified to date. Through single base mutations on each of the 8 nucleotide positions of the HBV PARP-1 binding site, significantly enhanced transcriptional activation was achieved with the sequence “ACATCAAA”. The characterization of the PARP-1 transcriptional activation motif should help in elucidating the extent of PARP-1 sequence-dependent transcriptional regulation, which would prove useful in the understanding of PARP-1 dependent diseases including cancers, inflammation and hepatitis B [33, 36, 37, 53].

Interestingly, as opposed to binding DNA strand-breaks, PARP-1 binding to its recognition motif inhibits its catalytic activity (FIG. 6A). This suggests that DNA sequence-specific binding of PARP-1 is unlikely to be mediated by the DNA strand-break binding domains. Considering that PARP-1 enzymatic activation results in the auto-ADP-ribosylation hence repulsion from negatively-charge DNA [27, 55], the inhibition of PARP-1 enzymatic activity by motif recognition is a plausible pre-requisite for PARP-1 to effectively mediate its transcriptional activities. This has been demonstrated in a number of instances, such as the transcription of vimentin and in the transcriptional regulation at the T cell leukemia virus type I tax-responsive element [28, 29], where enzymatic inhibition of PARP-1 enhances transcription. PARP-1 sequence-dependent transcriptional inhibition such as at the BRCA2 promoter may also be modulated in a similar manner [74]. This limits the spectrum of diseases that conventional chemical inhibitors of PARP-1 can act on, as the condition of PARP-1 motif recognition dependent diseases would be potentially aggravated with their use.

Since PARP-1 enzymatic activity may be inhibited by motif recognition and sequestration, understanding how this may be achieved could lead to the development of a new class of PARP-1 inhibitors that act on allosteric sites of the enzyme. Furthermore, it suggests that the PARP-1 motif may itself be used as novel DNA PARP-1 inhibitor for the treatment of PARP-1 dependent diseases.

Using the treatment of cancer as an example, PARP-1 enzymatic inhibition would impair PARP-1 dependent DNA repair, sensitizing cancer cells to the cytotoxic effects of DNA damage inducers. However, this increases the nuclear pool of unmodified PARP-1 hence PARP-1 dependent transcription, resulting in potentially serious complications in other disorders that the patient also suffers from, where PARP-1 is required as a sequence-specific transcriptional regulator, such as in hepatitis B. In such case, inhibition of PARP-1 enzymatic activity increases HBV replication (FIG. 8A, FIG. 8B), further endangering the health of the patient. In contrast, the use of the PARP-1 motif as a DNA inhibitor of PARP-1 activity would not only achieve the desired effect of DNA damage repair impairment for cancer therapy (FIG. 7), sequestered unmodified PARP-1 cannot act on the HBV core promoter to bring about pgRNA synthesis hence HBV replication. This advantage of PARP-1 inhibition cum sequestration may be further extended to inflammatory disorders, reducing contraindications when PARP-1 is the therapeutic target of interest.

### Examples Part 3

### Protocols for Testing the Efficacy of DNA PARP-1 Inhibitor

### Example 22

### Efficacy of DNA PARP-1 Inhibitor in Controlling HBV Replication—HBV Animal Models

Human HBV preferentially infects humans and primates. Several animal models for HBV infection are available, such as non-human primates including chimpanzees, woodchucks and transgenic mice. These models are unable to recapitulate all the hallmarks of HBV infection in humans. Human-mouse chimeric liver models susceptible to HBV infection are also available, but laborious surgical procedures may have confounding effects that hinder data analysis. As such, the use of the transgenic mouse model is recommended for determining the effect of the DNA PARP-1 inhibitor on HBV replication.

### Example 23

### Efficacy of DNA PARP-1 Inhibitor in Controlling HBV Replication—Experimental Design

| | | Treatment | | Expected outcome | | |
|---|---|---|---|---|---|---|
| | | PARP-1 | Validated | | Serum | |
| | | inhib- | HBV anti- | Liver | HBV | Serum |
| Experiment | | itor | viral | damage | DNA | HBs |
| 1 | Vehicle | Vehicle | Vehicle | High | High | High |
| | control | | | | | |
| 2 | Positive | Vehicle | Yes | Low | Low | Low |
| | control | | | | | |
| 3 | Negative | Random | Vehicle | High | High | High |
| | control | DNA | | | | |
| | | sequence | | | | |
| 4 | Test | ACATCAAA | Vehicle | Low | Low | Low |
| | PARP-1 | | | | | |
| | inhibitor | | | | | |
| 5 | Test for | ACATCAAA | Yes | Very | Very | Very |
| | synergism | | | low | low | low |

Use at least 6 mice per treatment group. Short DNA duplex “ACATCAAA” sequence may be delivered into the liver using a variety of delivery vehicles and these include liposomes, polymers, liver-specific viral vectors, electroporation, ultrasound and even conjugation to hepatocyte penetrating peptides. The optimal mode of delivery and concentration of the DNA PARP-1 inhibitor used has to be empirically determined. To reduce confounding effects from experimental procedures, minimally invasive methods are recommended. Validated HBV anti-virals such as the immune modulator interferon-α and nucleoside or nucleotide analogues such as Lamivudine, Adefovir and Tenofovir may be used as a positive control.

1. Confirm HBV Replication in Transgenic Mice by Obtaining Liver Biopsy

Treat young adult mice with appropriate dose of anesthesia.

Clip abdominal fur and scrub mice with Betadyne followed by alcohol.

Cut ventral skin followed by peritoneum with sterile surgical equipment.

Cut half of right liver lobe with scissors sterilized in bead sterilizer at 250° C. while gently holding liver in place.

Cauterized liver should not be bleeding.

Close the peritoneum with one suture.

Rejoin ventral skin with wound clips.

Wipe wound with Betadyne.

Perform southern blot on liver biopsy to confirm presence of HBV DNA.

Histological analysis of biopsies should not reveal abnormalities.

Allow mice to recover for at least 3 weeks before treatment.

2. Pre-Treatment HBV Replication Indicators

Obtain small blood sample from HBV-positive mice 3 days prior to treatment.

Determine and record HBV DNA and HBs concentrations in the serum.

3. Drug Treatment

Randomize HBV-positive mice for treatments lasting 3 weeks.

Treat mice with prescribed regiment defined by the group assigned.

Repeat treatment when deemed necessary.

Observe mice for manifestation of toxic effects such as loss of appetite.

4. Data Collection

Collect small samples of blood once every 7 days.

Determine and record HBV DNA and HBs concentrations in the serum.

Determine the extent of liver damage with the use of a panel of liver damage indicators such as ALT.

Sacrifice the mice and perform histological analysis for signs of liver damage and HBV replication.

5. Analysis of Data

Calculate indicators of HBV replication and liver damage as follows:

$$\% Indicator change = \frac{Value for day d - Pre \text{-} treatment value}{Pre \text{-} treatment value} \times 100 \% for day d$$

Significantly reduced HBV replication accompanied by significant reduction in liver damage in treatment groups compared to vehicle controls indicate that the PARP-1 inhibitor is a potential candidate for the treatment of HBV infection.

### Example 24

### Efficacy of DNA PARP-1 Inhibitor in Cytotoxicity by DNA Damage Inducers—Cell Line Selection

Any cell with measurable PARP-1 enzymatic activity may be used. The effect of the PARP-1 inhibitor is expected to be enhanced in cells with defects in DNA repair, such as in BRCA negative or Ku70/80 deficient cell lines.

### Example 25

### Efficacy of DNA PARP-1 Inhibitor in Cytotoxicity by DNA Damage Inducers—Delivery Systems

The described PARP-1 inhibitor is a short, DNA duplex. An efficient means of introducing DNA sequences into the nuclei of cells may be used. These include the use of liposomes, polymers, viral vectors, electroporation, ultrasound and even conjugation to cell penetrating peptides. The mode of DNA delivery chosen is cell line dependent.

### Example 26

### Efficacy of DNA PARP-1 Inhibitor in Cytotoxicity by DNA Damage Inducers—Induction of DNA Damage and DNA Damage Assays

Any agent known to affect PARP-1 dependent DNA damage repair pathways, namely base-excision repair (hence single-strand break repair) and DNA double-strand break repair, may be used.

These agents include:

Ionizing radiation (UV-irradiation, X-rays and γ-irradiation)

DNA alkylating agents (Example: N-nitroso-N-methylurea)

DNA strand-break inducers (Examples: Etoposide, bleomycin)

The concentration in which these agents are used is cell-line dependent and requires optimization. The dose used should be sufficient to induce sub-lethal but measurable DNA damage. Evidence of DNA damage may be confirmed by COMET assays. This has to be performed before DNA damage-dependent cell death can occur, typically 8-24 hours after the induction of DNA damage.

### Example 27

### Efficacy of DNA PARP-1 Inhibitor in Cytotoxicity by DNA Damage Inducers—Assays for Cell Death

Cell death can be assayed in a number of ways, such as detecting for caspase activation and the expression of phosphatidyl-serine on the outer leaflet of the cell membrane. Assays dependent on measuring DNA damage, such as TUNEL, are not suitable. Cell death assays should be performed before visible cell death is observed. The optimal time for performing the cell death assays is cell line, inhibitor concentration, DNA damage inducer and assay dependent. These have to be determined through optimization. Assays performed after 96 hours of induced DNA damage is not recommended.

### Example 28

### Efficacy of DNA PARP-1 Inhibitor in Cytotoxicity by DNA Damage Inducers—Experimental Set-Up and Evaluation of Outcomes

An example of how the PARP-1 inhibitor may be demonstrated to show sensitization of DNA damage dependent cell death and its expected outcome is as outlined in the table below.

| | | Treatment | | Expected outcome | |
|---|---|---|---|---|---|
| | | | DNA | | % |
| | | PARP-1 | damage | DNA | Cell |
| Experiment | | inhibitor | inducer | damage | death |
| 1 | Background | None | None | None | Minimal |
| | control | | | | |
| 2 | Background | None | Yes | Low | Low |
| | control | | | | |
| 3 | Negative | Validated | None | None | Low |
| | control | PARP-1 | | | |
| | | inhibitor | | | |
| 4 | Negative | ACATCAAA | None | None | Low |
| | control | | | | |
| 5 | Positive | Validated | Yes | High | High |
| | control | PARP-1 | | | |
| | | inhibitor | | | |
| 6 | Test | ACATCAAA | Yes | High | High |
| | inhibitor | | | | |
| 7 | Specif- | Null | Yes | Low | Low |
| | icity | mutant | | | |
| | control | of PARP-1 | | | |
| | | inhibitor | | | |

The use of the adherent liver cell line, HepG2, liposome-mediated transfection and the DNA damage inducer N-nitroso-N-methylurea (NMU) will be illustrated. Cell death will be assayed by microscopic examination of percentage of phosphatidyl-serine positive cells. Experiments should be performed in replicates of at least three. A range of concentrations (0.5 nM to 500 nM) of the novel PARP-1 inhibitor should be tested. Any validated PARP-1 inhibitor such as NU1025 may be used as positive control.

1. Prepare Single-Cell Suspension:

Grow healthy HepG2 to a confluency of about 70-80%.

Remove culture medium. Gently wash cells twice with sufficient PBS.

Add sufficient trypsin and incubate in a humidified 37° C., 5% CO₂ incubator for 5 minutes.

Stop trypsinization with fresh culture medium.

Break cell clumps by pipetting.

Obtain single-cell suspensions by filtration through nylon mesh.

Perform viable cell count using 0.4% trypan blue and haemocytometer.

2. Prepare Mastermix of PARP-1 Inhibitor for Transfection:

Prepare the test duplex PARP-1 inhibitor 1000× stock in nuclease-free water.

Dilute enough PARP-1 inhibitor to 10× stock with 50 μl of OPTI-MEM® per well. Mix well.

Dilute 1.1 μl Lipofectamine 2000 (Invitrogen) per well with 50 μl OPTI-MEM®. Mix well.

Let the mixtures sit at room temperature for 5 minutes.

Mix the diluted inhibitor with the diluted Lipofectamine 2000.

Incubate at room temperature for 20 minutes.

3. Seed and Reverse Transfect Cells:

Re-suspend cells to homogeneity.

Prepare single-cell suspension of 3.0×10⁵ cell/ml of culture medium (with serum).

Mix the PARP-1 inhibitor with cell suspension in the ratio of 1:5.

Add 600 μl of mixture per well.

Observe cells under light microscope. Make sure cells are not clustered in any part of the well.

Return the cells to the humidified 37° C., 5% CO₂ incubator for 24 hours.

4. Induce DNA Damage:

Observe the cells 24 hours after transfection. There should be little sign of cell death.

Dilute the DNA damage inducer NMU in culture medium to the range of 0.005%-0.02% (v/v).

Add PARP-1 inhibitors or its appropriate vehicle control when necessary.

Gently remove the culture medium from each well.

Wash the cells gently twice with PBS.

Add 500 μl of diluted NMU per well.

Return the cells to the humidified 37° C., 5% CO₂ incubator.

Perform COMET assay to confirm DNA damage induction 16-24 hours after drug treatment.

5. Assay for Cell Death and DNA Damage

Cell death assays may be performed 18-72 hours after drug treatment.

Observe cells under light microscope. Most of the cells should still adhere to the well.

Gently remove culture medium.

Gently add appropriate amounts of diluted fluorescein-conjugated annexin V.

Incubate on ice in the dark for 10 minutes.

Remove annexin V and gently wash once with culture medium.

Gently add 500 μl culture medium per well.

Immediately perform microscopic examination of percentage annexin V-positive cells.

### Example 29

### Efficacy of DNA PARP-1 Inhibitor in Nude Mouse Cancer Model

As PARP-1 inhibitors are especially potent in cells with BRCA mutations, the use of the MX-1 xenograft in nude mice model is a good starting point for the demonstration of efficacy of the novel DNA duplex PARP-1 inhibitor. The experimental design and outcomes needed to support PARP-1 enzymatic inhibition and tumour regression with the tested inhibitor are tabulated below.

### Example 30

### Efficacy of DNA PARP-1 Inhibitor in Nude Mouse Cancer Model—Experimental Design

| | | Treatment | | Expected outcome | |
|---|---|---|---|---|---|
| | | | DNA | PARP-1 | |
| | | PARP-1 | damage | enzymatic | Tumour |
| Experiment | | inhibitor | inducer | activity | volume |
| 1 | No | Vehicle | Vehicle | High | Large |
| | treatment | | | | |
| | control | | | | |
| 2 | Vehicle | Vehicle | Yes | High | Interme- |
| | control | | | | diate- |
| | | | | | Large |
| 3 | Test | ACATCAAA | Vehicle | Low | Interme- |
| | inhibitor | | | | diate- |
| | toxicity | | | | Large |
| 4 | Test | ACATCAAA | Yes | Low | Small |
| | inhibitor | | | | |
| | efficacy | | | | |
| 5 | Positive | Validated | Yes | Low | Small |
| | control | PARP-1 | | | |
| | | inhibitor | | | |

Use at least 6 mice per treatment group. DNA damage inducers include strand break inducers such as bleomycin and ionizing radiation, DNA alkylating agents such as temozolomide and cyclophosphamide, as well as DNA cross-linkers such as cisplatin. The dose of the DNA damage inducer chosen should be sufficient to cause DNA damage but insufficient to cause significant tumour regression when used on its own. Validated PARP-1 inhibitors such as ABT-888 may be used as a positive control. Any means of delivering the short DNA duplex “ACATCAAA” sequence into tumour cells can be used as vehicle. These include liposomes, polymers, viral vectors, electroporation, ultrasound and even conjugation to cell penetrating peptides. The optimal mode of delivery and concentration of inhibitor used has to be empirically determined.

1. Propagate MX-1 Tumour in Female Nude Mice

Rapidly thaw MX-1 (BRCA-deficient human mammary tumour) fragments in 37° C. water bath.

Prepare ˜27 mm³ fragments with sterile blade.

Implant fragment using a sterile 25 mm diameter trocar under the right flank skin.

Monitor the size of the implanted tumour with calipers.

Sacrifice donor mouse when tumour reaches ˜10 mm by cervical dislocation.

Excise tumour with sterile forceps and scissors.

Remove necrotic material.

Cut ˜27 mm³ fragments with sterile blade.

Repeat implantation procedure twice to ensure stable growth of tumour.

2. Drug Treatment with MX-1 Xenografts

Remove a tumour from a donor mouse of mass ≧18 g.

Remove necrotic material.

Cut ˜8 mm³ fragments with sterile blade.

Implant fragment using a sterile 25 mm diameter trocar under the right flank skin.

Randomize mice with similar tumour sizes according to desired representative disease stage.

(Early stage: ˜46-150 mm³ tumours; Late stage: ˜300 mm³ tumours)

Weigh the mice and record their pre-treatment weights.

Inject the randomized treatment regiment at 10 ml/kg.

Repeat injections may be performed when deemed necessary.

3. Adverse Reactions

Check the mice for toxicity by taking their weights every 2 days.

Weight loss of ≧15% pre-treatment weights or abnormality in behavior (e.g. poor appetite) indicate toxicity.

4. Data Collection and Interpretation

Measure and record the size (volume) of the tumour with digital calipers every other day.

Multiple parameters can be used to evaluate the efficacy of the PARP-1 inhibitor for anti-tumour activity.

$$Percentage T \text{/} C = \frac{Median tumour volume with test inhibitor}{Median tumour volume of vehicle control} \times 100 \% for day d$$

Efficacy is demonstrated if the optimal value (minimal value) is ≦42% (NCI standard criteria)

$$Specific tumour growth delay ( SGD ) = \frac{ \begin{matrix}Difference in time taken between test and \\ vehicle groups for tumour to reach 1500 {mm}^{3}\end{matrix}}{Time taken for tumours in vehicle group to reach 1500 {mm}^{3}}$$

Efficacy is demonstrated if the value exceeds 1 (Langdon et al, 1994)

$$Percentage rAUC = \frac{ \begin{matrix}Area under tumour growth curve \\ of a tumour treated with test inhibitor\end{matrix}}{ \begin{matrix}Median area under growth curve \\ of a tumour from vehicle control mice\end{matrix}} \times 100 \%$$

Efficacy is demonstrated if values from the test inhibitor group are significantly smaller than that of the vehicle control group when compared using the Mann-Whitney Rank Sum Test.

### REFERENCES

1. Mohanty, S. R., S. S. Kupfer, and V. Khiani, Treatment of chronic hepatitis B. Nat Clin Pract Gastroenterol Hepatol, 2006. 3(8): p. 446-58.
2. Livingston, S. E., et al., Hepatitis B virus genotypes in Alaska Native people with hepatocellular carcinoma: preponderance of genotype F. J Infect Dis, 2007. 195(1): p. 5-11.
3. Nguyen, V. T., M. G. Law, and G. J. Dore, Hepatitis B-related hepatocellular carcinoma: epidemiological characteristics and disease burden. J Viral Hepat, 2009. 16(7): p. 453-63.
4. Sanchez-Tapias, J. M., et al., Influence of hepatitis B virus genotype on the long-term outcome of chronic hepatitis B in western patients. Gastroenterology, 2002. 123(6): p. 1848-56.
5. Organization, W.H. Hepatitis B (Fact Sheet number 204). 2008 [cited 2010 22 Jul.].
6. Seeger, C. and W. S. Mason, Hepatitis B virus biology. Microbiol Mol Biol Rev, 2000. 64(1): p. 51-68.
7. Will, H., et al., Replication strategy of human hepatitis B virus. J Virol, 1987. 61(3): p. 904-11.
8. Beck, J. and M. Nassal, Hepatitis B virus replication. World J Gastroenterol, 2007. 13(1): p. 48-64.
9. Rehermann, B. and M. Nascimbeni, Immunology of hepatitis B virus and hepatitis C virus infection. Nat Rev Immunol, 2005. 5(3): p. 215-29.
10. Wong, D. K., et al., Quantification of hepatitis B virus covalently closed circular DNA in patients with hepatocellular carcinoma. J Hepatol, 2006. 45(4): p. 553-9.
11. Zoulim, F., New insight on hepatitis B virus persistence from the study of intrahepatic viral cccDNA. J Hepatol, 2005. 42(3): p. 302-8.
12. Tiollais, P., C. Pourcel, and A. Dejean, The hepatitis B virus. Nature, 1985. 317(6037): p. 489-95.
13. Lo, W. Y. and L. P. Ting, Repression of enhancer II activity by a negative regulatory element in the hepatitis B virus genome. J Virol, 1994. 68(3): p. 1758-64.
14. Kramvis, A. and M. C. Kew, The core promoter of hepatitis B virus. J Viral Hepat, 1999. 6(6): p. 415-27.
15. Ishida, H., et al., Identification of multiple transcription factors, HLF, FTF, and E4BP4, controlling hepatitis B virus enhancer II. J Virol, 2000. 74(3): p. 1241-51.
16. Raney, A. K., et al., Members of the nuclear receptor superfamily regulate transcription from the hepatitis B virus nucleocapsid promoter. J Virol, 1997. 71(2): p. 1058-71.
17. Zhang, P., A. K. Raney, and A. McLachlan, Characterization of functional Sp1 transcription factor binding sites in the hepatitis B virus nucleocapsid promoter. J Virol, 1993. 67(3): p. 1472-81.
18. Sun, C. T., et al., Transcription repression of human hepatitis B virus genes by negative regulatory element-binding protein/SON. J Biol Chem, 2001. 276(26): p. 24059-67.
19. Buckwold, V. E., M. Chen, and J. H. Ou, Interaction of transcription factors RFX1 and MIBP1 with the gamma motif of the negative regulatory element of the hepatitis B virus core promoter. Virology, 1997. 227(2): p. 515-8.
20. Su, H. and J. K. Yee, Regulation of hepatitis B virus gene expression by its two enhancers. Proc Natl Acad Sci USA, 1992. 89(7): p. 2708-12.
21. Moolla, N., M. Kew, and P. Arbuthnot, Regulatory elements of hepatitis B virus transcription. J Viral Hepat, 2002. 9(5): p. 323-31.
22. Yu, X. and J. E. Mertz, Differential regulation of the pre-C and pregenomic promoters of human hepatitis B virus by members of the nuclear receptor superfamily. J Virol, 1997. 71(12): p. 9366-74.
23. Shall, S. and G. de Murcia, Poly(ADP-ribose) polymerase-1: what have we learned from the deficient mouse model? Mutat Res, 2000. 460(1): p. 1-15.
24. Jagtap, P. and C. Szabo, Poly(ADP-ribose) polymerase and the therapeutic effects of its inhibitors. Nat Rev Drug Discov, 2005. 4(5): p. 421-40.
25. Kim, M. Y., T. Zhang, and W. L. Kraus, Poly(ADP-ribosyl)ation by PARP-1: ‘PAR-laying’ NAD+ into a nuclear signal. Genes Dev, 2005. 19(17): p. 1951-67.
26. Zaniolo, K., et al., Regulation of poly(ADP-ribose) polymerase-1 (PARP-1) gene expression through the post-translational modification of Sp1: a nuclear target protein of PARP-1. BMC Mol Biol, 2007. 8: p. 96.
27. Ogata, N., et al., Poly(ADP-ribose) synthetase, a main acceptor of poly(ADP-ribose) in isolated nuclei. J Biol Chem, 1981. 256(9): p. 4135-7.
28. Chu, S., et al., Poly(ADP-ribose) polymerase-1 regulates vimentin expression in lung cancer cells. Am J Physiol Lung Cell Mol Physiol, 2007. 293(5): p. L1127-34.
29. Zhang, Z., et al., Sequence-specific binding of poly(ADP-ribose) polymerase-1 to the human T cell leukemia virus type-I tax responsive element. Virology, 2002. 296(1): p. 107-16.
30. Loseva, O., et al., PARP-3 is a mono-ADP-ribosylase that activates PARP-1 in the absence of DNA. J Biol Chem. 285(11): p. 8054-60.
31. Szabo, C., P. Pacher, and R. A. Swanson, Novel modulators of poly(ADP-ribose) polymerase. Trends Pharmacol Sci, 2006. 27(12): p. 626-30.
32. Cohen-Armon, M., et al., DNA-independent PARP-1 activation by phosphorylated ERK2 increases Elk1 activity: a link to histone acetylation. Mol Cell, 2007. 25(2): p. 297-308.
33. Rouleau, M., et al., PARP inhibition: PARP1 and beyond. Nat Rev Cancer. 10(4): p. 293-301.
34. Bryant, H. E., et al., Specific killing of BRCA2-deficient tumours with inhibitors of poly(ADP-ribose) polymerase. Nature, 2005. 434(7035): p. 913-7.
35. Wang, X., et al., Haploinsufficiency of Parp1 accelerates Brca1-associated centrosome amplification, telomere shortening, genetic instability, apoptosis, and embryonic lethality. Cell Death Differ, 2007. 14(5): p. 924-31.
36. Peralta-Leal, A., et al., PARP inhibitors: new partners in the therapy of cancer and inflammatory diseases. Free Radic Biol Med, 2009. 47(1): p. 13-26.
37. Plummer, E. R., Inhibition of poly(ADP-ribose) polymerase in cancer. Curr Opin Pharmacol, 2006. 6(4): p. 364-8.
38. Ng, L. F., et al., Host heterogeneous ribonucleoprotein K (hnRNP K) as a potential target to suppress hepatitis B virus replication. PLoS Med, 2005. 2(7): p. e163.
39. Hassa, P. O., et al., Nuclear ADP-ribosylation reactions in mammalian cells: where are we today and where are we going? Microbiol Mol Biol Rev, 2006. 70(3): p. 789-829.
40. Smith, S., The world according to PARP. Trends Biochem Sci, 2001. 26(3): p. 174-9.
41. D'Amours, D., et al., Poly(ADP-ribosyl)ation reactions in the regulation of nuclear functions. Biochem J, 1999. 342 (Pt 2): p. 249-68.
42. Huang, D., et al., PARP-1 suppresses adiponectin expression through poly(ADP-ribosyl)ation of PPAR gamma in cardiac fibroblasts. Cardiovasc Res, 2009. 81(1): p. 98-107.
43. Zhao, F., et al., Cellular DNA repair cofactors affecting hepatitis B virus infection and replication. World J Gastroenterol, 2008. 14(32): p. 5059-65.
44. Kedar, P. S., et al., Interaction between PARP-1 and ATR in mouse fibroblasts is blocked by PARP inhibition. DNA Repair (Amst), 2008. 7(11): p. 1787-98.
45. Trenz, K., et al., ATM and ATR promote Mre11 dependent restart of collapsed replication forks and prevent accumulation of DNA breaks. EMBO J, 2006. 25(8): p. 1764-74.
46. Erdelyi, K., et al., Pathophysiologic role of oxidative stress-induced poly(ADP-ribose) polymerase-1 activation: focus on cell death and transcriptional regulation. Cell Mol Life Sci, 2005. 62(7-8): p. 751-9.
47. Ahel, D., et al., Poly(ADP-ribose)-dependent regulation of DNA repair by the chromatin remodeling enzyme ALC1. Science, 2009. 325(5945): p. 1240-3.
48. Woodhouse, B. C. and G. L. Dianov, Poly ADP-ribose polymerase-1: an international molecule of mystery. DNA Repair (Amst), 2008. 7(7): p. 1077-86.
49. Huber, A., et al., PARP-1, PARP-2 and ATM in the DNA damage response: functional synergy in mouse development. DNA Repair (Amst), 2004. 3(8-9): p. 1103-8.
50. Haince, J. F., et al., Ataxia telangiectasia mutated (ATM) signaling network is modulated by a novel poly(ADP-ribose)-dependent pathway in the early response to DNA-damaging agents. J Biol Chem, 2007. 282(22): p. 16441-53.
51. Hegde, M. L., T. K. Hazra, and S. Mitra, Early steps in the DNA base excision/single-strand interruption repair pathway in mammalian cells. Cell Res, 2008. 18(1): p. 27-47.
52. Tong, W. M., et al., Synergistic role of Ku80 and poly(ADP-ribose) polymerase in suppressing chromosomal aberrations and liver cancer formation. Cancer Res, 2002. 62(23): p. 6990-6.
53. Plummer, E. R. and H. Calvert, Targeting poly(ADP-ribose) polymerase: a two-armed strategy for cancer therapy. Clin Cancer Res, 2007. 13(21): p. 6252-6.
54. Mitchell, J., G. C. Smith, and N. J. Curtin, Poly(ADP-Ribose) polymerase-1 and DNA-dependent protein kinase have equivalent roles in double strand break repair following ionizing radiation. Int J Radiat Oncol Biol Phys, 2009. 75(5): p. 1520-7.
55. Altmeyer, M., et al., Molecular mechanism of poly(ADP-ribosylation by PARP1 and identification of lysine residues as ADP-ribose acceptor sites. Nucleic Acids Res, 2009. 37(11): p. 3723-38.
56. Frizzell, K. M., et al., Global analysis of transcriptional regulation by poly(ADP-ribose) polymerase-1 and poly(ADP-ribose) glycohydrolase in MCF-7 human breast cancer cells. J Biol Chem, 2009. 284(49): p. 33926-38.
57. Simbulan-Rosenthal, C. M., et al., Misregulation of gene expression in primary fibroblasts lacking poly(ADP-ribose) polymerase. Proc Natl Acad Sci USA, 2000. 97(21): p. 11274-9.
58. Ju, B. G., et al., A topoisomerase IIbeta-mediated dsDNA break required for regulated transcription. Science, 2006. 312(5781): p. 1798-802.
59. Krishnakumar, R., et al., Reciprocal binding of PARP-1 and histone H1 at promoters specifies transcriptional outcomes. Science, 2008. 319(5864): p. 819-21.
60. Hassa, P. O. and M. O. Hottiger, The functional role of poly(ADP-ribose)polymerase 1 as novel coactivator of NF-kappaB in inflammatory disorders. Cell Mol Life Sci, 2002. 59(9): p. 1534-53.
61. Rouleau, M., R. A. Aubin, and G. G. Poirier, Poly(ADP-ribosyl)ated chromatin domains: access granted. J Cell Sci, 2004. 117(Pt 6): p. 815-25.
62. Stilmann, M., et al., A nuclear poly(ADP-ribose)-dependent signalosome confers DNA damage-induced IkappaB kinase activation. Mol Cell, 2009. 36(3): p. 365-78.
63. Hassa, P. O., et al., Acetylation of poly(ADP-ribose) polymerase-1 by p300/CREB-binding protein regulates coactivation of NF-kappaB-dependent transcription. J Biol Chem, 2005. 280(49): p. 40450-64.
64. Alvarez-Gonzalez, R., Genomic maintenance: the p53 poly(ADP-ribosyl)ation connection. Sci STKE, 2007. 2007(415): p. pe68.
65. Kanai, M., et al., Inhibition of Crm1-p53 interaction and nuclear export of p53 by poly(ADP-ribosyl)ation. Nat Cell Biol, 2007. 9(10): p. 1175-83.
66. Maruyama, T., et al., Txk, a member of the non-receptor tyrosine kinase of the Tec family, forms a complex with poly(ADP-ribose) polymerase 1 and elongation factor 1alpha and regulates interferon-gamma gene transcription in Th1 cells. Clin Exp Immunol, 2007. 147(1): p. 164-75.
67. Akiyama, T., et al., Activation of Reg gene, a gene for insulin producing beta-cell regeneration: poly(ADP-ribose) polymerase binds Reg promoter and regulates the transcription by autopoly(ADP-ribosyl)ation. Proc Natl Acad Sci USA, 2001. 98(1): p. 48-53.
68. Ambrose, H. E., et al., Poly-(ADP-ribose) polymerase-1 (Parp-1) binds in a sequence-specific manner at the Bcl-6 locus and contributes to the regulation of Bcl-6 transcription. Oncogene, 2007. 26(42): p. 6244-52.
69. Pottier, N., et al., Expression of SMARCB1 modulates steroid sensitivity in human lymphoblastoid cells: identification of a promoter SNP that alters PARP1 binding and SMARCB1 expression. Hum Mol Genet, 2007. 16(19): p. 2261-71.
70. Butler, A. J. and C. P. Ordahl, Poly(ADP-ribose) polymerase binds with transcription enhancer factor 1 to MCAT1 elements to regulate muscle-specific transcription. Mol Cell Biol, 1999. 19(1): p. 296-306.
71. Plaza, S., et al., Involvement of poly (ADP-ribose)-polymerase in the Pax-6 gene regulation in neuroretina. Oncogene, 1999. 18(4): p. 1041-51.
72. Vidakovic, M., et al., PARP-1 expression in the mouse is controlled by an autoregulatory loop: PARP-1 binding to an upstream S/MAR element and to a novel recognition motif in its promoter suppresses transcription. J Mol Biol, 2009. 388(4): p. 730-50.
73. Kang, X., et al., The septic shock-associated IL-10-1082 A>G polymorphism mediates allele-specific transcription via poly(ADP-Ribose) polymerase 1 in macrophages engulfing apoptotic cells. J Immunol. 184(7): p. 3718-24.
74. Wang, J., et al., Poly(ADP-ribose) polymerase-1 down-regulates BRCA2 expression through the BRCA2 promoter. J Biol Chem, 2008. 283(52): p. 36249-56.
75. Grote, J., et al., Identification of poly(ADP-ribose)polymerase-1 and Ku70/Ku80 as transcriptional regulators of S100A9 gene expression. BMC Mol Biol, 2006. 7: p. 48.
76. Amiri, K. I., et al., Differential regulation of CXC ligand 1 transcription in melanoma cell lines by poly(ADP-ribose) polymerase-1. Oncogene, 2006. 25(59): p. 7714-22.
77. Si, H., S. C. Verma, and E. S. Robertson, Proteomic analysis of the Kaposi's sarcoma-associated herpesvirus terminal repeat element binding proteins. J Virol, 2006. 80(18): p. 9017-30.
78. Ono, T., et al., Positive transcriptional regulation of the human micro opioid receptor gene by poly(ADP-ribose) polymerase-1 and increase of its DNA binding affinity based on polymorphism of G-172->T. J Biol Chem, 2009. 284(30): p. 20175-83.
79. Nyquist, P., J. Zhang, and T. J. De Graba, The −928 G/C and −362 G/C single-nucleotide polymorphisms in the promoter of MCP-1: Increased transcriptional activity and novel binding sites. Cerebrovasc Dis. 29(3): p. 242-7.
80. Harrop, R., J. John, and M. W. Carroll, Recombinant viral vectors: cancer vaccines. Adv Drug Deliv Rev, 2006. 58(8): p. 931-47.
81. Singh, R. and K. Kostarelos, Designer adenoviruses for nanomedicine and nanodiagnostics. Trends Biotechnol, 2009. 27(4): p. 220-9.
82. Kutzler, M. A. and D. B. Weiner, DNA vaccines: ready for prime time? Nat Rev Genet, 2008. 9(10): p. 776-88.
Alkhatib, H. M., Chen, D., Cherney, B., Bhatia, K., Notario, V., Giri, C., Stein, G., Slattery, E., Roeder, R. G., Smulson, M. E. Cloning and expression of cDNA for human poly(ADP-ribose) polymerase. Proc. Nat. Acad. Sci. 84: 1224-1228, 1987.
Ambrose, H. E., Willimott, S., Beswick, R. W., Dantzer, F., Menissier de Murcia, J., Yelamos, J., Wagner, S. D. Poly(ADP-ribose) polymerase-1 (Parp-1)-deficient mice demonstrate abnormal antibody responses. Immunology 127: 178-186, 2008.
Andrabi, S. A., Kim, N. S., Yu, S.-W., Wang, H., Koh, D. W., Sasaki, M., Klaus, J. A., Otsuka, T., Zhang, Z., Koehler, R. C., Hurn, P. D., Poirier, G. G., Dawson, V. L., Dawson, T. M. Poly(ADP-ribose) (PAR) polymer is a death signal. Proc. Nat. Acad. Sci. 103: 18308-18313, 2006.
Auer, B., Nagl, U., Herzog, H., Schneider, R., Schweiger, M. Human nuclear NAD+ ADP-ribosyltransferase(polymerizing): organization of the gene. DNA 8: 575-580, 1989.
Baumgartner, M., Schneider, R., Auer, B., Herzog, H., Schweiger, M., Hirsch-Kauffmann, M. Fluorescence in situ mapping of the human nuclear NAD+ ADP-ribosyltransferase gene (ADPRT) and two secondary sites to human chromosomal bands 1q42, 13q34, and 14q24. Cytogenet. Cell Genet. 61: 172-174, 1992.
Bhatia, K. G., Cherney, B. W., Huppi, K., Magrath, I. T., Cossman, J., Sausville, E., Barriga, F., Johnson, B., Gause, B., Bonney, G., Neequayi, J., DeBernardi, M., Smulson, M. A deletion linked to a poly(ADP-ribose) polymerase gene on chromosome 13q33-qter occurs frequently in the normal black population as well as in multiple tumor DNA. Cancer Res. 50: 5406-5413, 1990.
Bryant, H. E., Schultz, N., Thomas, H. D., Parker, K. M., Flower, D., Lopez, E., Kyle, S., Meuth, M., Curtin, N. J., Helleday, T. Specific killing of BRCA2-deficient tumours with inhibitors of poly(ADP-ribose) polymerase. Nature 434: 913-916, 2005. (Note: Addendum: Nature 447: 346 only, 2007.)
Chang, P., Jacobson, M. K., Mitchison, T. J. Poly(ADP-ribose) is required for spindle assembly and structure. Nature 432: 645-649, 2004.
Cherney, B. W., McBride, O. W., Chen, D., Alkhatib, H., Bhatia, K., Hensley, P., Smulson, M. E. cDNA sequence, protein structure, and chromosomal location of the human gene for poly(ADP-ribose) polymerase. Proc. Nat. Acad. Sci. 84: 8370-8374, 1987.
Cohen-Armon, M., Visochek, L., Katzoff, A., Levitan, D., Susswein, A. J., Klein, R., Valbrun, M., Schwartz, J. H. Long-term memory requires polyADP-ribosylation. Science 304: 1820-1822, 2004.
d'Adda di Fagagna, F., Hande, M. P., Tong, W.-M., Lansdorp, P. M., Wang, Z.-Q., Jackson, S. P. Functions of poly(ADP-ribose) polymerase in controlling telomere length and chromosomal stability. Nature Genet. 23: 76-80, 1999.
Deschenes, F., Massip, L., Garand, C., Lebel, M. In vivo misregulation of genes involved in apoptosis, development and oxidative stress in mice lacking both functional Werner syndrome protein and poly(ADP-ribose) polymerase-1. Hum. Molec. Genet. 14: 3293-3308, 2005.
Doll, J. A., Suarez, B. K., Donis-Keller, H. Association between prostate cancer in black Americans and an allele of the PADPRP pseudogene locus on chromosome 13. Am. J. Hum. Genet. 58: 425-428, 1996.
Farmer, H., McCabe, N., Lord, C. J., Tutt, A. N. J., Johnson, D. A., Richardson, T. B., Santarosa, M., Dillon, K. J., Hickson, I., Knights, C., Martin, N. M. B., Jackson, S. P., Smith, G. C. M., Ashworth, A. Targeting the DNA repair defect in BRCA mutant cells as a therapeutic strategy. Nature 434: 917-921, 2005.
Flick, K., Schneider, R., Auer, B., Hirsch-Kauffmann, M., Schweiger, M. No abnormalities in the NAD(+) ADP-ribosyltransferase (polymerizing) gene of transformed cells from a Fanconi's anemia patient. Hum. Genet. 89: 690-691, 1992.
Fong, P. C., Boss, D. S., Yap, T. A., Tutt, A., Wu, P., Mergui-Roelvink, M., Mortimer, P., Swaisland, H., Lau, A., O'Connor, M. J., Ashworth, A., Carmichael, J., Kaye, S. B., Schellens, J. H. M., de Bono, J. S. Inhibition of poly(ADP-ribose) polymerase in tumors from BRCA mutation carriers. New Eng. J. Med. 361: 123-134, 2009.
Grube, K., Burkle, A. Poly(ADP-ribose) polymerase activity in mononuclear leukocytes of 13 mammalian species correlates with species-specific life span. Proc. Nat. Acad. Sci. 89: 11759-11763, 1992.
Herzog, H., Zabel, B., Schneider, R., Auer, B., Hirsch-Kauffmann, M., Schweiger, M. Human NAD(+):protein ADP ribosyltransferase (ADPRT): synthesis of active enzyme in E. coli, sequence of its cDNA from HeLa cells and chromosomal localization. Biol. Chem. Hoppe Seyler 369: 836-837, 1988.
Herzog, H., Zabel, B. U., Schneider, R., Auer, B., Hirsch-Kauffmann, M., Schweiger, M. Human nuclear NAD(+) ADP-ribosyltransferase: localization of the gene on chromosome 1q41-q42 and expression of an active human enzyme in Escherichia coli. Proc. Nat. Acad. Sci. 86: 3514-3518, 1989.
Kim, M. Y., Mauro, S., Gevry, N., Lis, J. T., Kraus, W. L. NAD(+)-dependent modulation of chromatin structure and transcription by nucleosome binding properties of PARP-1. Cell 119: 803-814, 2004.
Krishnakumar, R., Gamble, M. J., Frizzel, K. M., Berrocal, J. G., Kininis, M., Kraus, W. L. Reciprocal binding of PARP-1 and histone H1 at promoters specifies transcriptional outcomes. Science 319: 819-821, 2008. PubMed: 18258916]
Kurosaki, T., Ushiro, H., Mitsuuchi, Y., Suzuki, S., Matsuda, M., Matsuda, Y., Katunuma, N., Kangawa, K., Matsuo, H., Hirose, T., Inayama, S., Shizuta, Y. Primary structure of human poly(ADP-ribose) synthetase as deduced from cDNA sequence. J. Biol. Chem. 262: 15990-15997, 1987.
Loetscher, P., Alvarez-Gonzalez, R., Althaus, F. R. Poly(ADP-ribose) may signal changing metabolic conditions to the chromatin of mammalian cells. Proc. Nat. Acad. Sci. 84: 1286-1289, 1987.
Lyn, D., Cherney, B. W., Lalande, M., Berenson, J. R., Lichtenstein, A., Lupold, S., Bhatia, K. G., Smulson, M. A duplicated region is responsible for the poly(ADP-ribose) polymerase polymorphism, on chromosome 13, associated with a predisposition to cancer. Am. J. Hum. Genet. 52: 124-134, 1993.
Mao, Z., Hine, C., Tian, X., Van Meter, M., Au, M., Vaidya, A., Seluanov, A., Gorbunova, V. SIRT6 promotes DNA repair under stress by activating PARP1. Science 332: 1443-1446, 2011.
McBride, O. W., Cherney, B., Takourdin, C., Smulson, M. E. Human poly(ADP-ribose) polymerase sequences are located on chromosomes 1, 13, and 14. Cytogenet. Cell Genet. 46: 659-660, 1987.
Midorikawa, R., Takei, Y., Hirokawa, N. KIF4 motor regulates activity-dependent neuronal survival by suppressing PARP-1 enzymatic activity. Cell 125: 371-383, 2006.
Nasta, F., Laudisi, F., Sambucci, M., Rosado, M. M., Pioli, C. Increased Foxp3+ regulatory T cells in poly(ADP-ribose) polymerase-1 deficiency. J. Immun. 184: 3470-3477, 2010.
Nicholson, D. W., Ali, A., Thornberry, N. A., Vaillancourt, J. P., Ding, C. K., Gallant, M., Gareau, Y., Griffin, P. R., Labelle, M., Lazebnik, Y. A., Munday, N. A., Raju, S. M., Smulson, M. E., Yamin, T.-T., Yu, V. L., Miller, D. K. Identification and inhibition of the ICE/CED-3 protease necessary for mammalian apoptosis. Nature 376: 37-43, 1995.
Pavri, R., Lewis, B., Kim, T.-K., Dilworth, F. J., Erdjument-Bromage, H., Tempst, P., de Murcia, G., Evans, R., Chambon, P., Reinberg, D. PARP-1 determines specificity in a retinoid signaling pathway via direct modulation of Mediator. Molec. Cell 18: 83-96, 2005.
Petrilli, V., Herceg, Z., Hassa, P. O., Patel, N. S. A., Di Paola, R., Cortes, U., Dugo, L., Filipe, H.-M., Thiemermann, C., Hottiger, M. O., Cuzzocrea, S., Wang, Z.-Q. Noncleavable poly(ADP-ribose) polymerase-1 regulates the inflammation response in mice. J. Clin. Invest. 114: 1072-1081, 2004.
Pieper, A. A., Brat, D. J., Krug, D. K., Watkins, C. C., Gupta, A., Blackshaw, S., Verma, A., Wang, Z.-Q., Snyder, S. H. Poly(ADP-ribose) polymerase-deficient mice are protected from streptozotocin-induced diabetes. Proc. Nat. Acad. Sci. 96: 3059-3064, 1999.
Schweiger, M., Auer, B., Burtscher, H. J., Hirsch-Kauffmann, M., Klocker, H., Schneider, R. DNA repair in human cells: biochemistry of the hereditary diseases Fanconi's anaemia and Cockayne syndrome. Europ. J. Biochem. 165: 235-242, 1987.
Simbulan-Rosenthal, C. M., Haddad, B. R., Rosenthal, D. S., Weaver, Z., Coleman, A., Luo, R., Young, H. M., Wang, Z.-Q., Ried, T., Smulson, M. E. Chromosomal aberrations in PARP −/− mice: genome stabilization in immortalized cells by reintroduction of poly(ADP-ribose) polymerase cDNA. Proc. Nat. Acad. Sci. 96: 13191-13196, 1999.
Smithies, O., Gregg, R. G., Boggs, S. S., Koralewski, M. A., Kucherlapati, R. S. Insertion of DNA sequences into the human chromosomal beta-globin locus by homologous recombination. Nature 317: 230-234, 1985.
Thomas, K. R., Capecchi, M. R. Site-directed mutagenesis by gene targeting in mouse embryo-derived stem cells. Cell 51: 503-512, 1987.
Thomas, K. R., Folger, K. R., Capecchi, M. R. High frequency targeting of genes to specific sites in the mammalian genome. Cell 44: 419-428, 1986.
Tomoda, T., Kurashige, T., Moriki, T., Yamamoto, H., Fujimoto, S., Taniguchi, T. Enhanced expression of poly(ADP-ribose) synthetase gene in malignant lymphoma. Am. J. Hemat. 37: 223-227, 1991.
Tulin, A., Spradling, A. Chromatin loosening by poly(ADP)-ribose polymerase (PARP) at Drosophila puff loci. Science 299: 560-562, 2003.
Vasquez, K. M., Marburger, K., Intody, Z., Wilson, J. H. Manipulating the mammalian genome by homologous recombination. Proc. Nat. Acad. Sci. 98: 8403-8410, 2001.
Yu, S.-W., Andrabi, S. A., Wang, H., Kim, N. S., Poirier, G. G., Dawson, T. M., Dawson, V. L. Apoptosis-inducing factor mediates poly(ADP-ribose) (PAR) polymer-induced cell death. Proc. Nat. Acad. Sci. 103: 18314-18319, 2006.
Yu, S.-W., Wang, H., Poitras, M. F., Coombs, C., Bowers, W. J., Federoff, H. J., Poirier, G. G., Dawson, T. M., Dawson, V. L. Mediation of poly(ADP-ribose) polymerase-1-dependent cell death by apoptosis-inducing factor. Science 297: 259-263, 2002.
Zabel, B. U., Herzog, H., Schneider, R., Auer, B., Hirsch-Kauffmann, M., Schweiger, M. Chromosomal sublocalization of the gene for human poly (ADP-ribose) polymerase (NAD+ ADP-ribosyltransferase) at 1q41-42. Cytogenet. Cell Genet. 51: 1115, 1989.

### Further Aspects

Further aspects and embodiments of the invention are now set out in the following numbered paragraphs; it is to be understood that the invention encompasses these aspects:

Paragraph 1. A method comprising exposing poly (ADP-ribose) polymerase to a nucleic acid comprising the sequence RNNWCAAA, in which R is G or A, N is independently T, C, G or A and W is T or A, in which the poly ADP-ribosylation activity of poly (ADP-ribose) polymerase is reduced as a result of the exposure.

Paragraph 2. A method according to Paragraph 1, in which N at position 3 is C, A or T, preferably A or T, more preferably T.

Paragraph 3. A method according to Paragraph 1 or 2, in which N at position 2 is C.

Paragraph 4. A method according to Paragraph 1, 2 or 3, in which W at position 4 is T.

Paragraph 5. A method according to any preceding Paragraph, in which R at position 1 is A.

Paragraph 6. A method according to any preceding Paragraph, in which the nucleic acid is 30 or fewer, 29 or fewer, 28 or fewer, 27 or fewer, 26 or fewer, 25 or fewer, 24 or fewer, 23 or fewer, 22 or fewer, 21 or fewer, 20 or fewer, 19 or fewer, 18 or fewer, 17 or fewer, 16 or fewer, 15 or fewer, 14 or fewer, 13 or fewer, 12 or fewer, 11 or fewer, 10 or fewer or 9 or fewer nucleotides long.

Paragraph 7. A method according to any preceding Paragraph, in which the nucleic acid has the sequence ACATCAAA or ACTTCAAA.

Paragraph 8. A method of reducing the ability of a cell to repair DNA damage, the method comprising reducing the activity of poly (ADP-ribose) polymerase by a method according to any preceding Paragraph.

Paragraph 9. A method of killing a cell such as a cancer cell, the method comprising reducing the activity of poly (ADP-ribose) polymerase by a method according to any of Paragraphs 1 to 7 in the presence of a cytotoxic agent.

Paragraph 10. A method according to Paragraph 9, in which the cell comprises a BRCA1 and BRCA2-deficient cancer cell.

Paragraph 11. A method according to Paragraph 9 or 10, in which the cytotoxic agent is selected from the group consisting of: (a) ionising radiation; (b) ABT-888 (Abbott), AG014699 (Pfizer), AZD2281 (olaparib, AstraZeneca), BSI-201 (Sanofi-Aventis), CEP-8983/CEP-9722 (prodrug, Cephalon) and MK-4877 (Merck); and (c) temozolomide, platins, cyclophosphamide, N-Methyl-N′-Nitro-N-Nitrosoguanidine (MNNG), topoisomerase I poisons, topotecan, oxaliplatin, gemcitabine and carboplatin.

Paragraph 12. A method for inhibiting hepatitis B virus (HBV) replication, the method comprising reducing the activity of poly (ADP-ribose) polymerase by a method according to any of Paragraphs 1 to 8.

Paragraph 13. A complex comprising poly (ADP-ribose) polymerase bound to a nucleic acid comprising the sequence RNNWCAAA, in which R is G or A, N is independently T, C, G or A and W is T or A.

Paragraph 14. A nucleic acid capable of specifically binding to poly (ADP-ribose) polymerase and reducing its poly ADP-ribosylation activity, the nucleic acid comprising the sequence RNNWCAAA, in which R is G or A, N is independently T, C, G or A and W is T or A.

Paragraph 15. A complex according to Paragraph 13 or a nucleic acid according to Paragraph 14, in which: (a) N at position 3 is C, A or T, preferably A or T, more preferably T; (b) N at position 2 is C; (c) W at position 4 is T; (d) R at position 1 is A; (e) the nucleic acid has the sequence ACATCAAA; or (f) the nucleic acid has the sequence ACTTCAAA.

Paragraph 16. A pharmaceutical composition comprising a nucleic acid according to Paragraph 14 or 15 together with a pharmaceutically acceptable excipient, diluent or carrier.

Paragraph 17. A nucleic acid according to any of Paragraphs 14, 15 or 16 for use in a method of inhibiting hepatitis B virus (HBV) replication.

Paragraph 18. A nucleic acid according to any of Paragraphs 14, 15 or 16 for use in a method of enhancing the cytotoxicity of ionising radiation or a drug.

Paragraph 19. A method of treating an individual suffering or suspected to be suffering from hepatitis B virus infection, the method comprising administering a therapeutically effective amount of a nucleic acid according to any of Paragraphs 14, 15 or 16.

Paragraph 19. A method of treating an individual suffering or suspected to be suffering from cancer, the method comprising administering a therapeutically effective amount of a nucleic acid according to any of Paragraphs 13, 14 or 15, optionally together with a cytotoxic drug or exposure to ionising radiation.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1-15. (canceled)

16. A composition comprising an isolated nucleic acid capable of specifically binding to poly (ADP-ribose) polymerase and reducing its poly ADP-ribosylation activity, the nucleic acid comprising the sequence RNNWCAAA, in which R is independently G or A, N is independently T, C, G or A and W is independently T or A.

17. The composition of claim 16, wherein
the N at position 3 is C, A, or T;
the N at position 2 is C;
the W at position 4 is T; or
the R at position 1 is A.

18. The composition of claim 16, wherein
the N at position 3 is A, or T;
the N at position 2 is C;
the W at position 4 is T; or
the R at position 1 is A.

19. The composition of claim 16, wherein
the N at position 3 is T;
the N at position 2 is C;
the W at position 4 is T; or
the R at position 1 is A.

20. The composition of claim 16, wherein the isolated nucleic acid comprises the sequence ACATCAAA or ACTTCAAA.

21. The composition of claim 16, further comprising a poly (ADP-ribose) polymerase bound to the isolated nucleic acid.

22. The composition of claim 16, further comprising a pharmaceutically acceptable excipient, diluent or carrier.

23. The composition of claim 22, further comprising a cytotoxic agent.

24. The composition of claim 23, wherein the cytotoxic agent is selected from the group consisting of:
ABT-888; AG014699; AZD2281; BSI-201; CEP-8983; CEP-9722; MK-4877; temozolomide; platins; cyclophosphamide; N-Methyl-N′-Nitro-N-Nitrosoguanidine (MNNG); topoisomerase I poisons; topotecan; oxaliplatin; gemcitabine; and carboplatin.

25. A method of treatment comprising administering the composition of claim 16 to a subject in need of a reduction of poly (ADP-ribose) polymerase.

26. The method of claim 25, wherein the subject is a subject in need of treatment for cancer.

27. The method of claim 26, wherein the cancer is selected from the group consisting of:
breast cancer; BRCA1-deficient breast cancer; BRCA2-deficient breast cancer; or BRCA1-/BRCA-2 deficient breast cancer.

28. The method of claim 25, wherein the subject is a subject in need of treatment for hepatitis B virus (HBV).

29. The method of claim 25, wherein the subject is a subject in need of a reduction of the ability of a cell to repair DNA damage.

30. The method of claim 29, wherein the subject has been administered a cytotoxic agent.

31. The method of claim 25, wherein the subject is further administered a cytotoxic agent.

32. The method of claim 31, wherein the cytotoxic agent is selected from the group consisting of:
ionising radiation; ABT-888; AG014699; AZD2281; BSI-201; CEP-8983; CEP-9722; MK-4877; temozolomide; platins; cyclophosphamide; N-Methyl-N′-Nitro-N-Nitrosoguanidine (MNNG); topoisomerase I poisons; topotecan; oxaliplatin; gemcitabine; and carboplatin.

33. A method of reducing the poly ADP-ribosylation activity of poly (ADP-ribose) polymerase, the method comprising contacting the polymerase with the composition of claim 16.

34. A method of identifying a molecule for use in the treatment or prevention of hepatitis B or cancer, the method comprising
contacting poly (ADP-ribose) polymerase with a composition of claim 16 in the presence of a candidate molecule;
and detecting a decrease in binding of the nucleic acid of the composition to poly (ADP-ribose) polymerase as compared to in the absence of the candidate molecule;
wherein a decrease in binding of the nucleic acid of the composition to poly (ADP-ribose) polymerase as compared to in the absence of the candidate molecule indicates the candidate molecule is useful in the treatment or prevention of hepatitis B or cancer.
